(19) United States
(12) Patent Application Publication
(10) Pub. No.: US 2025/0352672 A1
(43) Pub. Date: Nov. 20, 2025

(54) METHOD FOR TREATMENT OF MYOTONIC DYSTROPHY COMBINING PROTEIN EXPRESSION AND RNA INTERFERENCE VECTOR DELIVERY WITH TISSUE DETARGETING

(71) Applicant: University of Washington, Seattle, WA (US)

(72) Inventors: Joel R. Chamberlain, Seattle, WA (US); Matthew R. Karolak, Seattle, WA (US)

(73) Assignee: University of Washington, Seattle, WA (US)

(21) Appl. No.: 18/864,633

(22) PCT Filed: May 12, 2023
(86) PCT No.: PCT/US2023/066938

§ 371 Date: Nov. 11, 2024

Related U.S. Application Data
(60) Provisional application No. 63/341,866, filed on May 13, 2022.

(51) Int. Cl. A61K 48/00; A61K 38/17; A61P 21/00; C12N 15/113; C12N 15/86

(52) U.S. Cl. CPC A61K 48/0058; A61K 38/1709; A61P 21/00; C12N 15/1137; C12N 15/86; C12N 2310/14; C12N 2310/3519; C12N 2750/14143; C12Y 207/11001

(57) ABSTRACT

The disclosure features compositions and methods for the treatment of trinucleotide repeat expansion disorders. The compositions described herein that may be used to treat such disorders include at least one nucleic acid construct comprising a first nucleic acid sequence. In some embodiments, the first nucleic acid sequence encodes a therapeutic protein. In some embodiments, the first nucleic acid sequence encodes a MBNL protein. In some embodiments, the first nucleic acid sequence encodes MBNL1 protein. The composition may comprise at least one nucleic acid construct comprising a second nucleic acid. In some embodiments, the second nucleic acid sequence encodes an interfering RNA construct that suppresses the expression of RNA transcripts containing aberrantly expanded repeat regions. Disclosed herein are also methods of increasing the presence of functional muscleblind-like protein (MBNL) in the nucleus of a cell with expression control in tissue types and methods of treating muscular dystrophy or spliceopathy using the compositions disclosed herein.

### CROSS-REFERENCE(S) TO RELATED APPLICATION(S)

This application claims the benefit of U.S. Provisional Application Nos. 63/341,866 filed May 13, 2022, the disclosure of which is hereby expressly incorporated herein by reference in its entirety.

### STATEMENT OF GOVERNMENT LICENSE RIGHTS

This invention was made with government support under Grant No. NIH 5 P50 AR065139-06, awarded by the National Institutes of Health. The government has certain rights in the invention.

### STATEMENT REGARDING SEQUENCE LISTING

The sequence listing associated with this application is provided in XML format in lieu of a paper copy and is hereby incorporated by reference into the specification. The name of the XML file containing the sequence listing is 3915-P1261WOUW_Seq_List_20230510.xml. The XML file is 60 KB; was created on May 10, 2023; and is being submitted via Patent Center with the filing of the specification.

### BACKGROUND

Myotonic dystrophy type 1 (DM1) is a dominant genetic disease with an adult-onset muscular dystrophy characterized by muscle weakness and stiffening (myotonia). Other features of the disease result from multisystem effects of gene mutation include somnolence (excessive sleepiness), a reduction in executive functioning, gastrointestinal complications, infertility, and cataract formation.

DM1 is caused by expression of an expanded CTG microsatellite repeat in the 3′ UTR of the dystrophia myotonica protein kinase (or DM protein kinase) (DMPK) gene. Repeat expansions greater than 50 result in the adult form of the disease. This trinucleotide repeat expansion results in spliceopathy and the expression of toxic gain-of-function RNA that forms ribonuclear foci and disrupts normal activities of RNA-binding proteins belonging to the MBNL and CELF families. Mutant DMPK transcripts in skeletal muscle, heart, and brain tissue are retained in the cell nucleus in microscopically visible ribonuclear foci, which are the most prominent histopathological hallmark of the disease. CUG expansions fold into stable double-stranded stem-loop structures with U-U mismatches with a strong affinity for proteins of the Muscleblind-like (MBNL) family, leading to sequestration and therefore depletion of these proteins. Loss of functional MBNL proteins contributes to pathology associated with myotonic dystrophy.

Specifically, spliceopathy is attributed to the sequestration of muscle blind like protein 1 (MBNL1) by the mutant DMPK mRNA leading to an upregulation of CELF1. Changes in alternative splicing, translation, localization, and mRNA stability due to sequestration of muscle blind like (MBNL) proteins and up-regulation of Elav-like family member (CELF1) are key to DM1 pathology. MBNL1 is involved in an estimated 1000 splicing events, but when bound and sequestered by the mutant DMPK mRNA in DM1, it no longer directs normal splicing of its target mRNAs. Loss of functional MBNL1 and an increase in CUG-binding protein 1 (CUGBP1), Elav-like family member (or CELF1), contribute to a shift in splicing from adult to fetal isoforms of unrelated gene pre-mRNAs in DM1 and mRNA repression and decay, respectively. The second form of myotonic dystrophy, DM2, is similarly caused by a DNA repeat expansion, but in an intron of the CCHC-type zinc finger nucleic acid binding protein (CNBP) gene. Therefore, the DMPK or CNBP gene microsatellite repeat expansion leads to cell toxicity with changes in activity of proteins involved in mRNA stability and processing, primarily by a MBNL1 loss-of-function disease mechanism.

Accordingly, despite the advances in the art, a need remains for methods and compositions that can regulate and control the levels of factors influencing myotonic dystrophy disease in a manner that prevents toxicity and/or damage to other organs, for e.g., cardiotoxicity, and effectively treats the disease. The present disclosure addresses these and related needs.

### SUMMARY

Described herein are compositions and methods useful for regulating and/or controlling the levels of factors involved in the etiology of myotonic dystrophy and for treating disorders associated with the imbalance of these factors. Specifically, the present disclosure provides therapeutic compositions that are useful in treating myotonic dystrophy and related disorders without the associated cardiotoxicity. Exemplary factors involved in the etiology of myotonic dystrophy include but are not limited to DMPK, MBNL, CUGBP1, CNBP, and CELF1. In some embodiments, the DMPK comprises an expanded CTG microsatellite repeat in the 3′ UTR of the DMPK gene. In some embodiments, the CNBP comprises a mutant CNBP comprising CCTG repeat expansions in the CNBP gene.

The compositions described herein that may be used to treat such disorders include at least one nucleic acid construct comprising a first nucleic acid sequence. In some embodiments, the first nucleic acid sequence encodes a therapeutic protein. In some embodiments, the first nucleic acid sequence encodes a MBNL protein. In some embodiments, the first nucleic acid sequence encodes MBNL1 protein.

The composition may comprise at least one nucleic acid construct comprising a second nucleic acid. In some embodiments, the second nucleic acid sequence encodes an interfering RNA construct that suppresses the expression of RNA transcripts containing aberrantly expanded repeat regions, such as siRNA, miRNA, and shRNA constructs that anneal to portions of nuclear-retained, repeat-expanded RNA transcripts, and promote the degradation of these pathological transcripts by way of various cellular processes. In some embodiments, the second nucleic acid sequence encodes a siRNA or a miRNA targeting DMPK. In some embodiments, the second nucleic acid sequence encodes a siRNA or a miRNA that hybridizes to an mRNA encoding a dystrophia myotonica protein kinase (DMPK) comprising expanded repeat regions.

In some embodiments, the composition comprises at least one nucleic acid construct comprising a first nucleic acid sequence and a second nucleic acid sequence. In some embodiments, the composition comprises at least one nucleic acid construct comprising a first nucleic acid sequence encoding a functional MBNL protein, and a second nucleic acid sequence encoding an interfering RNA targeting DMPK. In some embodiments, the first nucleic acid sequence encodes a functional MBNL1 protein.

In an embodiment, the composition may comprise at least one nucleic acid construct comprising a third nucleic acid sequence. In some embodiments, the third nucleic acid sequence encodes a regulatory element useful for controlling/regulating and/or directing tissue-specific expression of the at least one nucleic acid construct described herein. In some embodiments, the regulatory element is useful in controlling and/or directing tissue-specific expression of the therapeutic protein. In some embodiments, the regulatory element comprises a binding site or a target site for a cardiac miRNA. In some embodiments, the first nucleic acid sequence is operatively linked to the third nucleic acid sequence that encodes the binding site for a cardiac miRNA. In some embodiments, the cardiac miRNA is a miRNA expressed in cardiac muscle cells. In some embodiments, the cardiac miRNA is a miRNA expressed exclusively or predominantly in cardiac muscle cells. In some embodiments, the cardiac miRNA is miR208a.

In some embodiments, the composition comprises at least one nucleic acid construct comprising a first nucleic acid sequence, a second nucleic acid, and a third nucleic acid sequence. In some embodiments, the composition comprises at least one nucleic acid construct comprising a first nucleic acid sequence encoding a functional MBNL protein, a second nucleic acid sequence encoding an interfering RNA targeting DMPK, and a third nucleic acid sequence encoding a regulatory element. In some embodiments, the first nucleic acid sequence encodes a functional MBNL1 protein. In some embodiments, the regulatory element is a binding site for a cardiac miRNA. In some embodiments, the cardiac miRNA is a miRNA expressed exclusively or predominantly in cardiac muscle cells. In some embodiments, the first nucleic acid sequence is operatively linked to the third nucleic acid sequence.

In some embodiments, the composition comprises at least one nucleic acid construct comprising a first nucleic acid sequence and a third nucleic acid sequence. In some embodiments, the composition comprises at least one nucleic acid construct comprising a first nucleic acid sequence encoding a functional MBNL protein and a third nucleic acid sequence encoding a regulatory element. In some embodiments, the first nucleic acid sequence encodes a functional MBNL1 protein. In some embodiments, the regulatory element is a binding or target site for a cardiac miRNA. In some embodiments, the cardiac miRNA is a miRNA expressed exclusively or predominantly in cardiac muscle cells. In some embodiments, the first nucleic acid sequence is operatively linked to the third nucleic acid sequence.

In an embodiment, the composition may comprise at least one nucleic acid construct comprising a fourth nucleic acid sequence. In some embodiments, the fourth nucleic acid sequence encodes a chimeric intron with beta-globin (b-globin or β-globin) and immunoglobulin sequences. In some embodiments, the chimeric intron serves as an MBLN1 binding site. In some embodiments, the chimeric intron serves to autoregulate MBNL1 expression. In some embodiments, the chimeric intron serves to enhance MBLN1 expression.

In some embodiments, the composition comprises at least one nucleic acid construct comprising a first nucleic acid sequence, a second nucleic acid, a third nucleic acid, and a fourth nucleic acid. In some embodiments, the composition comprises at least one nucleic acid construct comprising a first nucleic acid sequence encoding a functional MBNL1 protein, a second nucleic acid sequence encoding an interfering RNA targeting DMPK, a third nucleic acid sequence encoding a regulatory element, and a fourth nucleic acid sequence encoding a chimeric intron with beta-globin (b-globin or β-globin) and immunoglobulin sequences.

In some embodiments, the chimeric intron serves as an MBLN1 binding site. In some embodiments, the chimeric intron serves to autoregulate MBNL1 expression. In some embodiments, the chimeric intron serves to enhance MBLN1 expression. In some embodiments, the regulatory element is a binding site for a cardiac miRNA. In some embodiments, the cardiac miRNA is a miRNA expressed exclusively or predominantly in cardiac muscle cells. In some embodiments, the first nucleic acid sequence is operatively linked to the third nucleic acid sequence. In some embodiments, the composition comprises at least one nucleic acid construct comprising the first nucleic acid sequence operatively linked to the fourth nucleic acid sequence.

In some embodiments, the composition comprises at least one nucleic acid construct comprising a first nucleic acid sequence, a third nucleic acid, and a fourth nucleic acid. In some embodiments, the first nucleic acid sequence encodes a functional MBNL protein. In some embodiments, the composition comprises at least one nucleic acid construct comprising a first nucleic acid sequence encoding a functional MBNL1 protein, a third nucleic acid sequence comprising a regulatory element, and a fourth nucleic acid sequence encoding a chimeric intron with beta-globin (b-globin or β-globin) and immunoglobulin sequences. In some embodiments, the chimeric intron serves as an MBLN1 binding site. In some embodiments, the chimeric intron serves to autoregulate MBNL1 expression. In some embodiments, the chimeric intron serves to enhance MBLN1 expression. In some embodiments, the regulatory element is a binding or target site for a cardiac miRNA. In some embodiments, the cardiac miRNA is a miRNA expressed exclusively or predominantly in cardiac muscle cells. In some embodiments, the first nucleic acid sequence is operatively linked to the third nucleic acid sequence. In some embodiments, the first nucleic acid sequence is operatively linked to the fourth nucleic acid sequence.

In some embodiments, the first nucleic acid sequence is operatively linked to the third nucleic acid sequence and the fourth nucleic acid sequence.

The compositions described herein may also comprise expression vectors comprising the at least one nucleic acid construct. The present disclosure additionally features compositions comprising vectors, such as viral vectors, encoding the at least one nucleic acid construct disclosed herein. Exemplary viral vectors described herein include but are not limited to adeno-associated viral (AAV) vectors, such as pseudotyped AAV2/8 and AAV2/9 vectors, and more recently derived myotropic AAV vectors.

In some embodiments, the first nucleic acid sequence (and optionally third and/or fourth nucleic acid sequences) and the second nucleic acid sequence are present on separate expression vector constructs. In some embodiments, the first nucleic acid sequence (and optionally third and/or fourth nucleic acid sequences) and the second nucleic acid sequence are present on the same expression vector construct. In some embodiments, the first nucleic acid sequence (and optionally third and/or fourth nucleic acid sequences) and the second nucleic acid sequence are operatively linked to the same promoter. Exemplary promoters include RNA Pol III (or Pol 3) and RNA Pol II (or Pol 3) promoters. In some embodiments, the promoter is a U6 promoter. In some embodiments, the promoter is a CK8e. In some embodiments, the first nucleic acid sequence (and optionally third and/or fourth nucleic acid sequences) and the second nucleic acid sequence are operatively linked to separate promoter sequences. In some embodiments, the first nucleic acid sequence (and optionally third and/or fourth nucleic acid sequences) are operatively linked to a CK8e promoter sequence and the second nucleic acid sequence is operatively linked to an RNA Pol III promoter (e.g., U6 promoter) sequence or RNA Pol II promoter (e.g., CK8) sequence.

The methods described herein include a method of increasing the presence of functional muscleblind-like protein (MBNL) in the nucleus of a cell. In some embodiments, the method comprises contacting the cell with at least one of the compositions disclosed herein. In some embodiments, the method comprises contacting the cell with a composition comprising at least one nucleic acid construct of the present disclosure. In some embodiments, the at least one nucleic acid construct comprises a first nucleic acid sequence encoding MBNL. In some embodiments, the first nucleic acid sequence encodes a functional MBNL1 protein.

In some embodiments, the method further comprises contacting the cell with a composition comprising at least one nucleic acid construct comprising the second nucleic acid. In some embodiments, the second nucleic acid sequence encodes an interfering RNA construct. In some embodiment, the interfering RNA construct suppresses or inhibits the expression of RNA transcripts containing aberrantly expanded repeat regions, such as siRNA, miRNA, and shRNA constructs that anneal to portions of nuclear-retained, repeat-expanded RNA transcripts, and promote the degradation of these pathological transcripts by way of various cellular processes. In some embodiments, the second nucleic acid sequence encodes a siRNA or a miRNA that hybridizes to an mRNA encoding a dystrophia myotonica protein kinase (DMPK). In some embodiments, the DMPK comprises a DMPK transcript comprising aberrantly expanded repeat regions. In some embodiments, the method comprises contacting the cell with a composition comprising at least one nucleic acid construct comprising the first nucleic acid sequence and the second nucleic acid sequence. In some embodiment, the second nucleic acid sequence is embedded in the 3′UTR of the first nucleic acid sequence.

In some embodiments, the method comprises contacting the cell with a composition comprising at least one nucleic acid construct comprising the first nucleic acid sequence encoding a functional MBNL1 protein, and the third nucleic acid sequence of the present disclosure. In some embodiments, the first nucleic acid sequence is operatively linked to the third nucleic acid sequence.

In some embodiments, the method comprises contacting the cell with a composition comprising at least one nucleic acid construct comprising the first nucleic acid sequence encoding a functional MBNL1 protein, the third nucleic acid sequence, and the fourth nucleic acid sequence of the present disclosure. In some embodiments, the first nucleic acid sequence is operatively linked to the third and to the fourth nucleic acid sequences.

In some embodiments, the method further comprises contacting the cell with a composition comprising at least one nucleic acid construct comprising the second nucleic acid sequence encoding an interfering RNA construct. In some embodiment, the interfering RNA construct suppresses or inhibits the expression of RNA transcripts containing aberrantly expanded repeat regions, such as siRNA, miRNA, and shRNA constructs that anneal to portions of nuclear-retained, repeat-expanded RNA transcripts, and promote the degradation of these pathological transcripts by way of various cellular processes. In some embodiments, the second nucleic acid sequence encodes a siRNA or a miRNA that hybridizes to an mRNA encoding a dystrophia myotonica protein kinase (DMPK). In some embodiments, the DMPK comprises a DMPK transcript comprising aberrantly expanded repeat regions.

In some embodiments, the method comprises contacting the cell with a composition comprising an expression vector comprising at least one nucleic acid construct disclosed herein.

In some embodiments, the cell is a mammalian cell. In some embodiment, the cell is a human cell. In some embodiments, the cell is in vitro. In some embodiments, the cell is in vivo.

Also provided herein are methods for treating myotonic dystrophy. In some embodiments, the method comprises administering a therapeutically effective amount of at least one of the compositions disclosed herein to a subject. In some embodiments, the subject suffers from DM1. In some embodiments, the subject is human.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1B show an exemplary gene delivery cassette for co-expression of MBNL1 in skeletal muscle and modalities to control expression of MBNL1. Schematic illustration of exemplary DMPK targeting, synthetic microRNA30a (miR30a)-based RNAi gene expression cassettes using a muscle specific, Pol 2 promoter, CK8e (or other similar MSEC) and either a therapeutic gene (MBNL1) or a carrier gene (β-Actin) (FIG. 1A). Schematic illustration of alterative expression cassette designs that can be used depending on whether RNAi expression is potent enough for the desired therapeutic effect (FIG. 1B).

FIGS. 2A-2C show methods to control MBNL1 gene expression levels in skeletal muscle and heart in steps to combine MBNL1 expression and DMPK RNAi as a systemic therapy. Schematic representation of the MBNL1 gene with 5′ UTR, exons, and 3′UTR depicted as boxes with exons and their lengths in DNA basepairs in parentheses indicated below (FIG. 2A). Translation start and stop codon sequences are labeled at top. The cDNA used for MBNL1 expression, MBNL1 40 (isoform 40) is a prevalent isoform and does not include exons 7 and 9 as indicated. The absence of exon 7 reduces toxic DMPK mRNA expanded repeat binding and inclusion of exon 5 is known to direct nuclear localization. For MBNL1 expression restriction to skeletal muscle, an AAV vector expression cassette containing the target sequence of the cardiac, endogenously expressed microRNA208a (miR208a) was engineered (FIG. 2B). A gene cassette containing 3 sequential copies (with short spacer sequences between) of the miR208a target sequence for binding miR208a (referred to as miR208aTSx3), was inserted in the 3′UTR of the MBNL1 cDNA. MBNL1 cDNA expression was compared with or without the miR208aTSx3 (SEQ ID NOS: 3 and 4, respectively) to test for reduced expression in cardiac tissue (see following figures) following systemic administration. AAV-ACMV-Luciferase was used as a negative control vector (promoter removed from a CMV-Luciferase expression cassette). Schematic of the cardiac de-targeted version of the MBNL1 therapeutic cDNA expression cassette from FIG. 2B showing potential locations of inserting therapeutic RNAi cassettes targeting DMPK (sequences based on synthetic miRNAs, such as miR30a used routinely and in previous patent) (FIG. 2C). As indicated in FIG. 1B, RNAi cassettes could also be expressed from a separate Pol 2 (for example, CK8) or Pol 3 (For example, U6 promoter).

FIG. 3 shows the study design to evaluate AAV-mediated systemic expression of MBNL1 in wild type mice with and without cardiac restricted expression. The schematic shows the timeline and methods of evaluating the safety of expressing MBNL1 in mice following AAV6-mediated systemic delivery. Wild type male and female C57BL6J 3wk old mice were randomized into 3 groups of 5-7mice, weighed, and cardiac function analyzed by echocardiogram 1 week prior to treatment. At 4 weeks of age, each group was retro-orbitally infused with 7.5×10¹² vector genomes (vg) of one of the experimental AAV vectors: CK8-MBNL1, or CK8-MBNL1-miR208aTSx3, as well as ACMV-Luciferase (ACMV-Luc [negative control]; lacking promoter activity due to deletion of CMV promoter sequences) serving as a negative control vector. Mice were weighed weekly and cardiac function was analyzed by echocardiogram at 1-, 3-, 5-, and 7-weeks post infusion. Mice were euthanized after 8 weeks post infusion and tissues were harvested for analysis.

FIG. 4 shows survival assessment post treatment with MBNL1 and control vectors. Mice receiving AAV-ACMV-Luc and AAV-MBNL1miR208aTSx3 appeared heathy and lived to meet the study endpoint at 8 weeks. In contrast, AAV-CK8-MBNL1 injected mice showed a rapid decline in body condition at about 2 weeks post infusion. Poorly functioning mice, as determined to have met humane end point criteria by research staff in consultation with facility veterinary staff after the first mouse expired, were euthanized prior to the 8 weeks post injection study endpoint between 3-5 weeks post injection. Humane endpoint criteria included body condition scoring and echocardiogram evaluation.

FIGS. 5A-5B show body and heart weight monitoring with AAV-mediated systemic expression of MBNL1 in wild type mice with and without cardiac restriction. Body weight of mice receiving AAV-CK8-MBNL1 was significantly lower than mice receiving negative control vectors (AAV-ACMV-Luc) or cardiac detargeting vector (AAV-CK8-MBNL1-miR208aTSx3) (FIG. 5A). At study endpoint (8wks post infusion), there was no statistically significant difference in body weight of mice receiving control or cardiac detargeting vector including the miR208. Ventricle to total heart weight ratio of mice receiving AAV-CK8-MBNL1 was significantly reduced compared to mice receiving control and cardiac detargeting vector (FIG. 5B). Note that a second mouse died unexpectedly and was not assessed at endpoint in the AAV-CK3-MBNL1 cohort.

FIGS. 6A-6C show echocardiographic examination of AAV-mediated expression of MBNL1 with and without cardiac restriction. FIGS. 6A, 6B, and 6C: 1,3, and 7 weeks post treatment, respectively, showing echocardiogram (and movie of contraction) of short view of ventricle AND heartbeat trace static view for ACMV-Luc, MBNL1, and MBNL1-miR208aTSx3 side-by-side post-treatment (1 week), top row; all 3 at 3 weeks post-treatment below; and control and MBNL1-miR208aTSx3 at 7 weeks (Note: AAV-MBNL1 treated mice were euthanized between 3-5 weeks due to failure to thrive). Bradycardia (slow heart rate) observed was noted at right in FIG. 6B, as well as hypokinesis (reduced contractility).

FIGS. 7A-7B show immunoblot analysis and quantitation of MBNL1 protein expression levels of MBNL1, endogenous mouse Mbnl1 protein expression levels, and control vectors at study endpoint. Western blot analysis of human MBNL1 and endogenous mouse Mbnl1 protein expression levels in gastrocnemius muscle, heart, and liver of treated mice at 8 weeks post injection using a polyclonal anti-MBNL1 antibody (Cell Signaling) (FIG. 7A). Two representative mice per group are shown. GAPDH detection was used as a loading control for quantitation (FIG. 7A (Cont.)). Bar graph of MBNL1 levels normalized to GAPDH (set at 1) and quantified by pixel density of the chemiluminescent label detected. MBNL1 protein level was elevated 4-fold in heart in mice receiving AAV-CK8-MBNL1 vector and no upregulation in cardiac tissue was observed in mice receiving the MBNL1 expression limiting cardiac vector, AAV-CK8-MBNL1-miR208aTSx3 (FIG. 7B). A slight increase was observed in gastrocnemius of the MBNL1 and MBNL1miR208aTSx3 treated mice, but more samples from the cohorts are needed to assess significance. A modest increase in nuclear localization and function of MBNL1, is sufficient for achieving therapeutic efficacy.

FIGS. 8A-8B show striated muscle tissue structural analysis for assessment of AAV-mediated MBNL1 protein expression effects. Tissue histological staining with hematoxylin and eosin (H&E) of heart (FIG. 8A) and gastrocnemius muscle (gastroc) (FIG. 8B) shows cardiac dilation of the ventricle in transverse cryosections from the AAV-MBNL1 heart not seen in the AAV-MBNL1miR208aTSx3 and AAV-ACMV-Luc (negative control) treated muscle. Mice receiving AAV-CK8-MBNL1 appeared to have regions of necrosis and fibrosis in higher power view of cardiac tissue. Gross tissue morphology appeared normal in TA muscles of all groups.

FIG. 9 shows a schematic illustration of design of an exemplary embodiment disclosed herein to implement expression of functional MBNL1 and anti-DMPK miRNA in muscle cells with expression of a cardiac tissue-restricted miRNA binding site to specifically prevent translation of MBNL1 in the heart. The schematic illustrates the expression cassette/vector, the RNA transcribed therefrom, and resulting functionalities. The exemplary expression construct comprises a creatine kinase 8e promoter driving expression of a chimeric/hybrid intron consisting of human β-globin and immunoglobulin heavy chain sequences for high expression of MBNL1 cDNA. The MBNL1 3′ UTR is the site of the DMPK miR for RNAi sequence expression and the binding sites for three cardiac restricted endogenous miR208a sequences (miR208aTSx3) are placed at the end of the internal ribosome entry site (IRES). An IRES is shown between the 3′ UTR and the RNA polymerase II polyadenylation site (pA), remaining as a residual DNA element to provide additional sequence for the size requirement of the vector genome necessary for efficient vector preparation yields, acting as a spacer or filler. The pA sequence provides mRNA stability. In this expression vector iteration, the expression of an mRNA containing the translation coding sequence of MBNL1 is blocked in cardiac tissue with the expression of endogenous miR208a sequences that are generated by the endogenous RNAi pathway. The miR208a sequences bind to the target sites in the expressed mRNA containing the MBNL1 coding sequence to block translation. The mRNA is not degraded but serves as a platform for production of the DMPK miR sequences that are processed by the nuclear and cytoplasmic endogenous RNAi pathway proteins. Released DMPK miR sequences can associate with DMPK mRNA to either direct degradation of the transcript driven by highly homologous miRs or by binding and dissociation of MBNL1 proteins to allow nuclear or cytoplasmic degradation.

FIGS. 10A-10D show assessment of AAV-MBNL1 (all MBNL1 vectors with miR208aTSx3 sequences) expression with systemic delivery in the HSA^{LR} mouse model of DM1 and wild type mice. MBNL1 protein expression in the heart and quadricep of wild type (FIGS. 10A and 10C) and HSA^{LR} mice (FIGS. 10B and 10D) transduced with vectors comprising either CK8-intron-MBNL1 miR208a target sites (CK8-intron-MBNL1) (i.e., MBNL1 containing the chimeric β-globin/immunoglobulin gene intron), CK8-MBNL1 miR208a target sites (CK8-MBNL1; MBNL1 without the chimeric b-globin/immunoglobulin gene intron), and a control promoter less luciferase vector (ACMV-Luc). AAV-CK8-MBNL1-intron and AAV-CK8-MBNL1-NO intron vectors were injected into mice at a dose of 7.5×10¹² vg (vector genomes in viral capsids) at 4 weeks of age and analyzed 8 weeks post-vector delivery as done in previous experiment; GAPHD protein was detected as a control for quantitation for comparison of samples. Student t test with SEM for statistical analyses. Arrow indicates treated mouse with highest expression of MBNL1.

FIGS. 11A-11B show structural analysis of striated muscles following AAV-MBNL1 vector systemic delivery in the HSA^{LR} mouse model of DM1. Structural features were assessed with H&E staining of heart (FIG. 11A) and quadriceps muscle (FIG. 11B).

Muscle cryosections were stained and microscopic images were analyzed for signs of damage and response to damage indicated by central nuclei and infiltrating immune cells. Muscle appears similar in structure to control vector treated wild-type mice with no signs of damage and inflammation (FIGS. 8A-8B). Scale bar=50 um.

FIGS. 12A-12C show quantitation of alternative splicing changes as a readout of the effect of AAV-MBNL1 treatment on disease pathology. Splicing of Atp2a1 and Bin1 mRNAs isolated from the quadriceps muscles of AAV-MBNL1 treated HSA^{LR} mice were assessed by RT-PCR with splice forms evident on agarose gel separation (FIG. 12A), with quantitation shown in the graphs (FIGS. 12B and 12C). No changes in either mRNA splicing patterns were observed except in HSA^{LR} mice treated with the AAV-CK8-MBNL1-intron vector in a mouse with ˜4-fold upregulation of MBNL1 protein (western blot in FIG. 10B [indicated with arrow]). One mouse displayed a nearly complete correction of Atp2a1 splicing with inclusion of exon 22 and a shift in the less affected Bin1 splicing pattern to inclusion of exon 11. A=adult splice form; N=neonatal splice form. Student t test with SEM was used for statistical analyses.

FIGS. 13A-13B show DMPK miRNA activity quantitation in HEK293 and DM1 myogenic precursor cells. Evaluation of the gene silencing activity of U6 DMPK miRNAs (FIG. 13A). Candidate therapeutic miRNA expression cassettes 5 and 8 showed significant reduction of the endogenous DMPK mRNA 48 hrs after transfection of HEK293 cells with 1.5 ug of plasmid DNA compared to a control plasmid with no miRNA expression cassette. U6-miRNA plasmid and control plasmids were assayed with biological replicates n=8.

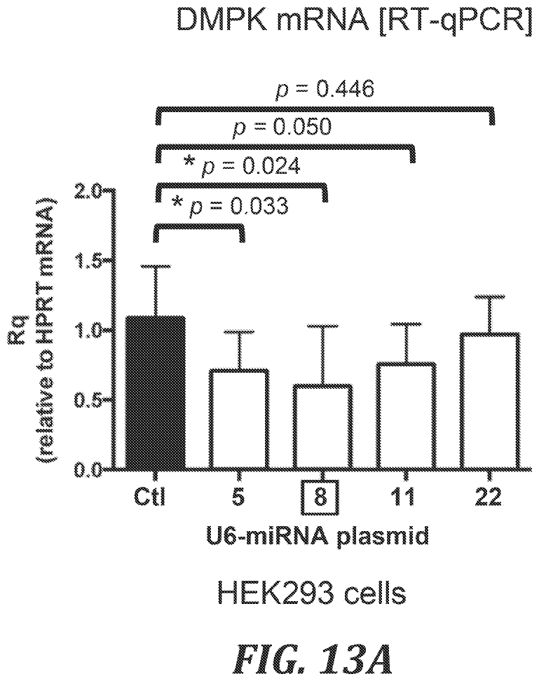

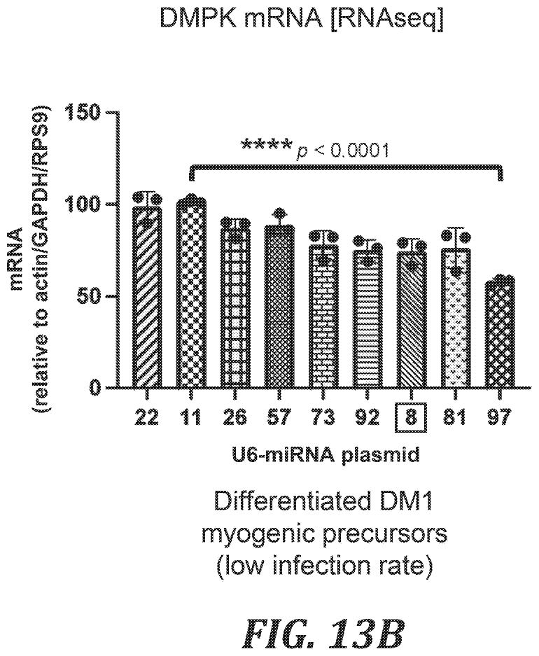

Candidate therapeutic miRNA expression cassettes were cloned into AAV plasmids and prepared as AAV6 vectors (FIG. 13B). DM1 myogenic precursor cells were infected with AAV6-DMPK miR vectors and RNA isolated from the cells were used for RNA sequencing (RNAseq; DESeq) analysis of DMPK mRNA reduction. DMPK miR97 (black checkered bar) reduced DMPK mRNA 43% compared to the least effective DMPK miR. DMPK mRNA levels were determined in comparison to 3 different internal control genes β-actin, GAPDH, and RPS9. Statistical analyses were performed using Student t-test *p<0.05 for A and 2-way Anova with multiple comparisons in B; error bars=SD.

### DETAILED DESCRIPTION

The inventors previously developed adeno-associated virus (AAV) vectors for delivery of gene sequences that will direct RNA degradation, termed RNA interference (RNAi), of a specific mRNA sequence due to extensive homology of the RNAi gene sequence to the disease-related mRNA target sequence. See U.S. patent application Ser. No. 17/054,474 (U.S. Patent Publication No. 202110269825), incorporated herein by reference in its entirety. The DMPK miR targets the disease-causing DMPK RNA (both alleles, i.e., with or without the expanded CTG repeat that leads to expression of long repeats containing CUG sequences in the RNA). The normal DMPK mRNA is also targeted, but therapeutic RNAi does not eliminate the targeted population of RNA completely.

The present disclosure is directed to the inventors' advancement of the RNAi-based DM1 therapy where DMPK RNAi (DMPK miR) for silencing, reducing, or inhibiting, expanded repeat DMPK mRNA, is combined with controlled expression of MBNL1 protein for treatment of myotonic dystrophy type 1 (DM1). The proof-of-concept design and approach is also adaptable for myotonic dystrophy type 2 (DM2). As described in more detail below, the inventors developed gene expression cassette components for myotonic dystrophy therapy to reduce the need for high level expression of either of the two therapeutic gene sequences, for example, including but not limited to, MBNL1; and interfering RNA targeting DMPK, alone and for tuning tissue expression. An exemplary gene expression vector/cassette includes but is not limited to the following components: 1) viral vector-based (e.g., AAV) delivery of muscleblind-like gene, MBNL1 and/or with MBNL2; 2) gene-embedded microRNA (miR) for RNAi retargeted destruction of the expanded repeat DMPK mRNA; 3) miR target sequence for a cardiac tissue restricted miR to limit expression in heart tissue. Thus, the exemplary gene expression cassette also contains a gene-embedded miR expression platform for production of interfering RNAs targeting DMPK mRNA, aimed at reducing the expanded repeat DMPK mRNA. Table 1 lists the amino acid sequences for the human MBNL1 protein isoform and for the Renilla luciferase -Firefly luciferase N-terminal fragment fusion protein reporter used in the present disclosure. The sequences for the exemplary expression cassettes/vectors used in the present disclosure are listed in Table 2.

MBNL1 and MBNL2 normally function in splicing sets of cellular pre-mRNAs and are less efficient because of their binding and inactivation by the disease-causing myotonica dystrophy protein kinase gene (DMPK) mRNA carrying an expanded microsatellite repeat (CTG for DM1 or CCTG for DM2). The inventors used a muscle restricted promoter, which could be altered to express in any or all DM affected tissues, to express an MBNL1 alone and/or with a MBNL2 bicistronic cDNA (MBNL1 and MBNL2 with an Internal Ribosome Entry Site sequence).

Overexpression of MBNL1 in striated muscle of normal mice has been demonstrated to be detrimental to the function of cardiac tissue. Cardiac toxicity including bradycardia and dilated cardiomyopathy with damaged cardiomyocytes is observed upon histological examination in mice overexpressing MBNL1 in striated muscle. In view of the foregoing, in addition to the MBNL genes for protein expression and the miR targeting DMPK for RNAi, the inventors added a third component, a cardiac microRNA binding site to prevent protein expression of MBNL1 in cardiac tissue to avoid the potential side effects of MBNL expression in the heart. Further, a fourth component comprising a nucleic acid sequence comprising a chimeric intron with beta-globin (b-globin or β-globin) and immunoglobulin domains was also included. The chimeric intron potentially serves as an MBLN1 binding site. It is also contemplated that the chimeric intron serves to autoregulate MBNL1 expression. In some embodiments, the chimeric intron serves to enhance MBLN1 expression.

TABLE 1

| SEQ ID NO: | SEQUENCE |
|---|---|
| 1 | MAVSVTPIRDTKWLTLEVCREFQRGTCSRPDTECKFAHPSKSCQ |
| MBNL1 | VENGRVIACFDSLKGRCSRENCKYLHPPPHLKTQLEINGRNNLIQ |
| Isoform 2 | QKNMAMLAQQMQLANAMMPGAPLQPVPMFSVAPSLATNASAA |
| (Homo sapiens) | AFNPYLGPVSPSLVPAEILPTAPMLVTGNPGVPVPAAAAAAAQKL |
| | MRTDRLEVCREYQRGNCNRGENDCRFAHPADSTMIDTNDNTVT |
| | VCMDYIKGRCSREKCKYFHPPAHLQAKIKAAQYQVNQAAAAQA |
| | AATAAAMGIPQAVLPPLPKRPALEKTNGATAVENTGIFQYQQAL |
| | ANMQLQQHTAFLPPVPMVHGATPATVSAATTSATSVPFAATATA |
| | NQIPIISAEHLTSHKYVTQM |
| 2 | MASKVYDPEQRKRMITGPQWWARCKQMNVLDSFINYYDSEKH |
| Renilla | AENAVIFLHGNAASSYLWRHVVPHIEPVARCIIPDLIGMGKSGKS |
| luciferase- | GNGSYRLLDHYKYLTAWFELLNLPKKIIFVGHDWGACLAFHYSY |
| Firefly | EHQDKIKAIVHAESVVDVIESWDEWPDIEEDIALIKSEEGEKMVL |
| luciferase N- | ENNFFVETMLPSKIMRKLEPEEFAAYLEPFKEKGEVRRPTLSWPR |
| terminal | EIPLVKGGKPDVVQIVRNYNAYLRASDDLPKMFIESDPGFFSNAI |
| fragment | VEGAKKFPNTEFVKVKGLHFSQEDAPDEMGKYIKSFVERVLKNE |
| fusion protein | Q |

TABLE 2
Exemplary Expression cassettes for myotonic dystrophy therapy

| SEQ ID NO: | SEQUENCE |
|---|---|
| 3 | cagctgcgcgctcgctcgctcactgaggccgcccgggcaaagcccgggcgtcgggcgacctttggtcg |
| CK8- | cccggcctca gtgagcgagc gagcgcgcag agagggagtg gccaactcca tcactaggggtt |
| MBNL1- | ccttgtagtt aatgattaac ccgccatgct acttatctac aaactctaga gcggccgcac gcgtacgcta |
| miR208aTSx3/ | gctagactag catgctgccc atgtaaggag gcaaggcctg gggacacccg agatgcctgg |
| pAAV-Ck8- | ttataattaa cccagacatg tggctgcccc ccccccccca acacctgctg cctctaaaaa |
| mbnl1_ | taaccctgca tgccatgttc ccggcgaagg gccagctgtc ccccgccagc tagactcagc |
| mir208a3Xts | acttagttta ggaaccagtg agcaagtcag cccttggggc agcccataca aggccatggg |
| (intron | gctgggcaag ctgcacgcct gggtccgggg tgggcacggt gcccgggcaa cgagctgaaa |
| bolded, | gctcatctgc tctcaggggc ccctccctgg ggacagcccc tcctggctag tcacaccctg |
| MBNL1 | taggctcctc tatataaccc aggggcacag gggctgccct cattctacca ccacctccac |
| inclusive of | agcacagaca gacactcagg agccagccag cgtatacgta cttctgacac aacagtctcg |
| stop codon | aacttaagct gcagaagttg gtcgtgaggc actgggcagg taagtatcaa ggttacaaga |
| underlined, | caggtttaaa cggagaccaa tagaaactgg gcttgtcgag acagagaaga ctcttgcgtt |
| and | tctgataggc acctattggt cttactgaca tccactttgc ctttctctcc acaggtgtcc |
| miR208aTSx3 | actcccagtt caattacagc tcttaaggct agagtaaccg gtgccaccat ggcagttagt |
| in | gttactccga tacgcgatac caagtggctc actctcgaag tctgtagaga atttcagcga |
| underlined | gggacctgct cccgacctga tactgaatgt aagtttgcgc acccatctaa atcatgtcaa |
| italics) | gtggagaacg gcagggtgat tgcatgtttc gacagtctca aaggccggtg ttcacgagag |
| | aattgcaagt atcttcatcc accaccgcac ctcaagactc agcttgaaat aaacggccgc |
| | aataacctta tacaacagaa gaacatggcc atgctggcac aacaaatgca attggccaac |
| | gcaatgatgc caggggcgcc cctgcaacca gttccaatgt tctcagttgc accatcactc |
| | gcaacaaatg catccgctgc tgccttcaac ccgtaccttg ggcccgtttc acccagcctt |
| | gtgcccgctg agattctccc tacagcgcct atgctggtaa |
| | cgggtaaccc tggtgtcccc gttcccgcag cagcggccgc agccgctcag aaattgatgc |
| | gcaccgacag attggaggta tgccgagagt accagcgcgg aaactgtaat agaggggaaa |
| | atgactgccg gtttgcccac ccagctgact caacaatgat tgacactaat gacaatacgg ttaccgtttg |
| | tatggactac ataaaaggac ggtgctcccg cgaaaaatgc aaatacttcc atcccccagc |
| | gcatcttcag gccaagatta aggcagccca ataccaagtc aaccaggcag cggctgcgca |
| | agcagcagcc acggcagcag caatgggcat tccgcaggca gtactcccac cccttccaaa |
| | gaggccggcg ctcgagaaga ccaatggtgc aacagctgtg tttaacacgg gtatctttca |
| | atatcagcaa gcacttgcga acatgcaact ccaacaacat actgcgtttt tgccaccagt |
| | cccaatggta catggcgcta caccggcgac agtaagtgct gcgacaacga gcgctacatc |
| | cgtgccattc gctgcgacag ctacagcgaa ccagatcccg atcatctccg cagagcactt |
| | gacttcccat aagtacgtta ctcagatgta gtccggagtt aaccctaggg gcgcgcccgc |
| | ccctctccct cccccccccc taacgttact ggccgaagcc gcttggaata aggccggtgt |
| | gcgtttgtct atatgttatt ttccaccata ttgccgtctt ttggcaatgt gagggcccgg aaacctggcc |
| | ctgtcttctt gacgagcatt cctaggggtc tttcccctct cgccaaagga atgcaaggtc tgttgaatgt |
| | cgtgaaggaa gcagttcctc tggaagcttc ttgaagacaa acaacgtctg tagcgaccct |
| | ttgcaggcag cggaaccccc cacctggcga caggtgcctc tgcggccaaa agccacgtgt |
| | ataagataca cctgcaaagg cggcacaacc ccagtgccac gttgtgagtt ggatagttgt |
| | ggaaagagtc aaatggctct cctcaagcgt attcaacaag gggctgaagg atgcccagaa |
| | ggtaccccat tgtatgggat ctgatctggg gcctcggtgc acatgcttta catgtgttta gtcgaggtta |
| | aaaaaacgtc taggcccccc gaaccacggg gacgtggttt tcctttgaaa aacacgatga |
| | taattgtaca cctgcaggca catcggtgga cctgagtcac aagctttttg ctcgtcttat gtcgaccctc |
| | gaggacaagc tttttgctcg tcttatgtcg acgacctgag tcacaagctt tttgctcgtc ttatcacatc |
| | ggtgcctgca ggtgtacaag ggaacttgtt tattgcagct tataatggtt acaaataaag caatagcatc |
| | acaaatttca caaataaagc atttttttca ctgcagatct gaattggccg tttgtagata agtagcatgg |
| | cgggttaatc attaactaca aggaacccct agtgatggag ttggccactc cctctctgcg |
| | cgctcgctcg ctcactgagg ccgggcgacc aaaggtcgcc cgacgcccgg gctttgcccg |
| | ggcggcctca gtgagcgagc gagcgcgcag ctggcgtaat agcgaagagg cccgcaccga |
| | tcgcccttcc caacagttgc gcagcctgaa tggcgaatgg cgattccgtt gcaatggctg |
| | gcggtaatat tgttctggat attaccagca aggccgatag tttgagttct tctactcagg caagtgatgt |
| | tattactaat caaagaagta ttgcgacaac ggttaatttg cgtgatggac agactctttt actcggtggc |
| | ctcactgatt ataaaaacac ttctcaggat tctggcgtac cgttcctgtc taaaatccct ttaatcggcc |
| | tcctgtttag ctcccgctct gattctaacg aggaaagcac gttatacgtg ctcgtcaaag caaccatagt |
| | acgcgccctg tagcggcgca ttaagcgcgg cgggtgtggt ggttacgcgc agcgtgaccg |
| | ctacacttgc cagcgcccta gcgcccgctc ctttcgcttt cttcccttcc tttctcgcca cgttcgccgg |
| | ctttccccgt caagctctaa atcgggggct ccctttaggg ttccgattta gtgctttacg gcacctcgac |
| | cccaaaaaac ttgattaggg tgatggttca cgtagtgggc catcgccctg atagacggtt tttcgccctt |
| | tgacgttgga gtccacgttc tttaatagtg gactcttgtt ccaaactgga acaacactca accctatctc |
| | ggtctattct tttgatttat aagggatttt gccgatttcg gcctattggt taaaaaatga gctgatttaa |
| | caaaaattta acgcgaattt taacaaaata ttaacgttta caatttaaat atttgcttat acaatcttcc |
| | tgtttttggg gcttttctga ttatcaaccg gggtacatat gattgacatg ctagttttac gattaccgtt |
| | catcgattta gggttaggcg ttttgcgctg cttcgcgatg tacgggccag atatacgcgt tgacattgat |
| | tattgactag ttattaatag taatcaatta cggggtcatt agttcatagc ccatatatgg agttccgcgt |
| | tacataactt acggtaaatg gcccgcctgg ctgaccgccc aacgaccccc gcccattgac |
| | gtcaataatg acgtatgttc ccatagtaac gccaataggg actttccatt gacgtcaatg ggtggactat |
| | ttacggtaaa ctgcccactt ggcagtacat caagtgtatc atatgccaag tacgccccct attgacgtca |
| | atgacggtaa atggcccgcc tggcattatg cccagtacat gaccttatgg gactttccta cttggcagta |
| | catctacgta ttagtcatcg ctattaccat ggtgatgcgg ttttggcagt acatcaatgg gcgtggatag |
| | cggtttgact cacggggatt tccaagtctc caccccattg acgtcaatgg gagtttgttt tggcaccaaa |
| | atcaacggga ctttccaaaa tgtcgtaaca actccgcccc attgacgcaa atgggcggta |
| | ggcgtgtacg gtggggtact tatataaggg ggtgggggcg cgttcgtcct cagtcgcgat |
| | cgaacactcg agccgagcag acgtgcctac ggaccgtaat acgactcact atagggaagc |
| | ttggcattcc ggtactgttg gtaaagccac catggcttcc aaggtgtacg accccgagca |
| | acgcaaacgc atgatcactg ggcctcagtg gtgggctcgc tgcaagcaaa tgaacgtgct |
| | ggactccttc atcaactact atgattccga gaagcacgcc gagaacgccg tgatttttct gcatggtaac |
| | gctgcctcca gctacctgtg gaggcacgtc gtgcctcaca tcgagcccgt ggctagatgc |
| | atcatccctg atctgatcgg aatgggtaag tccggcaaga gcgggaatgg ctcatatcgc |
| | ctcctggatc actacaagta cctcaccgct tggttcgagc tgctgaacct tccaaagaaa atcatctttg |
| | tgggccacga ctggggggct tgtctggcct ttcactactc ctacgagcac caagacaaga |
| | tcaaggccat cgtccatgct gagagtgtcg tggacgtgat cgagtcctgg gacgagtggc |
| | ctgacatcga ggaggatatc gccctgatca agagcgaaga gggcgagaaa atggtgcttg |
| | agaataactt cttcgtcgag accatgctcc caagcaagat catgcggaaa ctggagcctg |
| | aggagttcgc tgcctacctg gagccattca aggagaaggg cgaggttaga cggcctaccc |
| | tctcctggcc tcgcgagatc cctctcgtta agggaggcaa gcccgacgtc gtccagattg |
| | tccgcaacta caacgcctac cttcgggcca gcgacgatct gcctaagatg ttcatcgagt |
| | ccgaccctgg gttcttttcc aacgctattg tcgagggagc taagaagttc cctaacaccg |
| | agttcgtgaa ggtgaagg gcctccacttca gccaggagga cgctccagat gaaatgggta |
| | agtacatcaa gagcttcgtg gagcgcgtgc tgaagaacga gcagtaattc ttgccagcca |
| | tattctatag tgtcacctaa atgctagagc tcgctgatca gcctcgactg tgccttctag ctttcctaat |
| | tctgttgttt gcccctcccc cgtgccttcc ttgaccctgg aaggtgccac tcccactgtc gggggtgggg |
| | tagagggccc aaaatgagga aattgcatcg cattgtctga gtaggtgtca ttctattctg |
| | tggggcagga cagcaagggg gaggattggg aagacaatag caggcatgct ggggatgcgg |
| | tgggctctat ggcttctgag gcggaaagaa ccagctgggg ctcgaggggg atcgattctc |
| | ttgtttgctc cagactctca ggcaatgacc tgatagcctt tgtagagacc tctcaaaaat agctaccctc |
| | tccggcatga atttatcagc tagaacggtt gaatatcata ttgatggtga tttgactgtc tccggccttt |
| | ctcacccgtt tgaatcttta cctacacatt actcaggcat tgcatttaaa atatatgagg gttctaaaaa |
| | tttttatcct tgcgttgaaa taaaggcttc tcccgcaaaa gtattacagg gtcataatgt ttttggtaca |
| | accgatttag ctttatgctc tgaggcttta ttgcttaatt ttgctaattc tttgccttgc ctgtatgatt |
| | tattggatgt tggaattcct gatgcggtat tttctcctta cgcatctgtg cggtatttca caccgcatat |
| | ggtgcactct cagtacaatc tgctctgatg ccgcatagtt aagccagccc cgacacccgc |
| | caacacccgc tgacgcgccc tgacgggctt gtctgctccc ggcatccgct tacagacaag |
| | ctgtgaccgt ctccgggagc tgcatgtgtc agaggttttc accgtcatca ccgaaacgcg |
| | cgagacgaaa gggcctcgtg atacgcctat ttttataggt taatgtcatg ataataatgg tttcttagac |
| | gtcaggtggc acttttcggg gaaatgtgcg cggaacccct atttgtttat ttttctaaat acattcaaat |
| | atgtatccgc tcatgagaca ataaccctga taaatgcttc aataatattg aaaaaggaag agtatgagta |
| | ttcaacattt ccgtgtcgcc cttattccct tttttgcggc attttgcctt cctgtttttg ctcacccaga |
| | aacgctggtg aaagtaaaag atgctgaaga tcagttgggt gcacgagtgg gttacatcga |
| | actggatctc aacagcggta agatccttga gagttttcgc cccgaagaac gttttccaat gatgagcact |
| | tttaaagttc tgctatgtgg cgcggtatta tcccgtattg acgccgggca agagcaactc |
| | ggtcgccgca tacactattc tcagaatgac ttggttgagt actcaccagt cacagaaaag |
| | catcttacgg atggcatgac agtaagagaa ttatgcagtg ctgccataac catgagtgat |
| | aacactgcgg ccaacttact tctgacaacg atcggaggac cgaaggagct aaccgctttt |
| | ttgcacaaca tgggggatca tgtaactcgc cttgatcgtt gggaaccgga gctgaatgaa |
| | gccataccaa acgacgagcg tgacaccacg atgcctgtag caatggcaac aacgttgcgc |
| | aaactattaa ctggcgaact acttactcta gcttcccggc aacaattaat agactggatg |
| | gaggcggata aagttgcagg accacttctg cgctcggccc ttccggctgg ctggtttatt |
| | gctgataaat ctggagccgg tgagcgtggg tctcgcggta tcattgcagc actggggcca |
| | gatggtaagc cctcccgtat cgtagttatc tacacgacgg ggagtcaggc aactatggat |
| | gaacgaaata gacagatcgc tgagataggt gcctcactga ttaagcattg gtaactgtca |
| | gaccaagttt actcatatat actttagatt gatttaaaac ttcattttta atttaaaagg atctaggtga |
| | agatcctttt tgataatctc atgaccaaaa tcccttaacg tgagttttcg ttccactgag cgtcagaccc |
| | cgtagaaaag atcaaaggat cttcttgaga tccttttttt ctgcgcgtaa tctgctgctt gcaaacaaaa |
| | aaaccaccgc taccagcggt ggtttgtttg ccggatcaag agctaccaac tctttttccg aaggtaactg |
| | gcttcagcag agcgcagata ccaaatactg tccttctagt gtagccgtag ttaggccacc |
| | acttcaagaa ctctgtagca ccgcctacat acctcgctct gctaatcctg ttaccagtgg ctgctgccag |
| | tggcgataag tcgtgtctta ccgggttgga ctcaagacga tagttaccgg ataaggcgca |
| | gcggtcgggc tgaacggggg gttcgtgcac acagcccagc ttggagcgaa cgacctacac |
| | cgaactgaga tacctacagc gtgagctatg agaaagcgcc acgcttcccg aagggagaaa |
| | ggcggacagg tatccggtaa gcggcagggt cggaacagga gagcgcacga gggagcttcc |
| | agggggaaac gcctggtatc tttatagtcc tgtgggttt cgccacctct gacttgagcg tcgatttttg |
| | tgatgctcgt caggggggcg gagcctatgg aaaaacgcca gcaacgcggc ctttttacgg |
| | ttcctggcct tttgctggcc ttttgctcac atgttctttc ctgcgttatc ccctgattct gtggataacc |
| | gtattaccgc ctttgagtga gctgataccg ctcgccgcag ccgaacgacc gagcgcagcg |
| | agtcagtgag cgaggaagcg gaagagcgcc caatacgcaa accgcctctc cccgcgcgtt |
| | ggccgattca ttaatg |
| 4 | cagctgcgcg ctcgctcgct cactgaggcc gcccgggcaa agcccgggcg tcgggcgacc |
| AAV-CK8- | tttggtcgcc cggcctcagt gagcgagcga gcgcgcagag agggagtggc caactccatc |
| MBNL1 | actaggggtt ccttgtagtt aatgattaac ccgccatgct acttatctac aaactctaga |
| pAAv-Ck8- | gcggccgcac gcgtacgcta gctagactag catgctgccc atgtaaggag gcaaggcctg |
| mbnl1_ | gggacacccg agatgcctgg ttataattaa cccagacatg tggctgcccc ccccccccca |
| NOmiRtarget- | acacctgctg cctctaaaaa taaccctgca tgccatgttc ccggcgaagg gccagctgtc |
| Site (intron | ccccgccagc tagactcagc acttagttta ggaaccagtg agcaagtcag cccttggggc |
| bolded and | agcccataca aggccatggg gctgggcaag ctgcacgcct gggtccgggg tgggcacggt |
| MBNL1 | gcccgggcaa cgagctgaaa gctcatctgc tctcaggggc ccctccctgg ggacagcccc |
| inclusive of | tcctggctag tcacaccctg taggctcctc tatataaccc aggggcacag gggctgccct |
| stop codon | cattctacca ccacctccac agcacagaca gacactcagg agccagccag cgtatacgta |
| underlined) | cttctgacac aacagtctcg aacttaagct gcagaagttg gtcgtgaggc actgggcagg |
| | taagtatcaa ggttacaaga caggtttaaa cggagaccaa tagaaactgg gcttgtcgag |
| | acagagaaga ctcttgcgtt tctgataggc acctattggt cttactgaca tccactttgc |
| | ctttctctcc acaggtgtcc actcccagtt caattacagc tcttaaggct agagtaaccg |
| | gtgccaccat ggcagttagt gttactccga tacgcgatac caagtggctc actctcgaag |
| | tctgtagaga atttcagcga gggacctgct cccgacctga tactgaatgt aagtttgcgc |
| | acccatctaa atcatgtcaa gtggagaacg gcagggtgat tgcatgtttc gacagtctca |
| | aaggccggtg ttcacgagag aattgcaagt atcttcatcc accaccgcac ctcaagactc |
| | agcttgaaat aaacggccgc aataacctta tacaacagaa gaacatggcc atgctggcac |
| | aacaaatgca attggccaac gcaatgatgc caggggcgcc cctgcaacca gttccaatgt |
| | tctcagttgc accatcactc gcaacaaatg catccgctgc tgccttcaac ccgtaccttg |
| | ggcccgtttc acccagcctt gtgcccgctg agattctccc tacagcgcct atgctggtaa |
| | cgggtaaccc tggtgtcccc gttcccgcag cagcggccgc agccgctcag aaattgatgc |
| | gcaccgacag attggaggta tgccgagagt accagcgcgg aaactgtaat agaggggaaa |
| | atgactgccg gtttgcccac ccagctgact caacaatgat tgacactaat gacaatacgg |
| | ttaccgtttg tatggactac ataaaaggac ggtgctcccg cgaaaaatgc aaatacttcc |
| | atcccccagc gcatcttcag gccaagatta aggcagccca ataccaagtc aaccaggcag |
| | cggctgcgca agcagcagcc acggcagcag caatgggcat tccgcaggca gtactcccac |
| | cccttccaaa gaggccggcg ctcgagaaga ccaatggtgc aacagctgtg tttaacacgg |
| | gtatctttca atatcagcaa gcacttgcga acatgcaact ccaacaacat actgcgtttt |
| | tgccaccagt cccaatggta catggcgcta caccggcgac agtaagtgct gcgacaacga |
| | gcgctacatc cgtgccattc gctgcgacag ctacagcgaa ccagatcccg atcatctccg |
| | cagagcactt gacttcccat aagtacgtta ctcagatgta gtccggagtt aaccctaggg |
| | gcgcgcccgc ccctctccct cccccccccc taacgttact ggccgaagcc gcttggaata |
| | aggccggtgt gcgtttgtct atatgttatt ttccaccata ttgccgtctt ttggcaatgt |
| | gagggcccgg aaacctggcc ctgtcttctt gacgagcatt cctaggggtc tttcccctct |
| | cgccaaagga atgcaaggtc tgttgaatgt cgtgaaggaa gcagttcctc tggaagcttc |
| | ttgaagacaa acaacgtctg tagcgaccct ttgcaggcag cggaaccccc cacctggcga |
| | caggtgcctc tgcggccaaa agccacgtgt ataagataca cctgcaaagg cggcacaacc |
| | ccagtgccac gttgtgagtt ggatagttgt ggaaagagtc aaatggctct cctcaagcgt |
| | attcaacaag gggctgaagg atgcccagaa ggtaccccat tgtatgggat ctgatctggg |
| | gcctcggtgc acatgcttta catgtgttta gtcgaggtta aaaaaacgtc taggcccccc |
| | gaaccacggg gacgtggttt tcctttgaaa aacacgatga taattgtaca agggaacttg |
| | tttattgcag cttataatgg ttacaaataa agcaatagca tcacaaattt cacaaataaa |
| | gcattttttt cactgcagat ctgaattggc cgtttgtaga taagtagcat ggcgggttaa |
| | tcattaacta caaggaaccc ctagtgatgg agttggccac tccctctctg cgcgctcgct |
| | cgctcactga ggccgggcga ccaaaggtcg cccgacgccc gggctttgcc cgggcggcct |
| | cagtgagcga gcgagcgcgc agctggcgta atagcgaaga ggcccgcacc gatcgccctt |
| | cccaacagtt gcgcagcctg aatggcgaat ggcgattccg ttgcaatggc tggcggtaat |
| | attgttctgg atattaccag caaggccgat agtttgagtt cttctactca ggcaagtgat |
| | gttattacta atcaaagaag tattgcgaca acggttaatt tgcgtgatgg acagactctt |
| | ttactcggtg gcctcactga ttataaaaac acttctcagg attctggcgt accgttcctg |
| | tctaaaatcc ctttaatcgg cctcctgttt agctcccgct ctgattctaa cgaggaaagc |
| | acgttatacg tgctcgtcaa agcaaccata gtacgcgccc tgtagcggcg cattaagcgc |
| | ggcgggtgtg gtggttacgc gcagcgtgac cgctacactt gccagcgccc tagcgcccgc |
| | tcctttcgct ttcttccctt cctttctcgc cacgttcgcc ggctttcccc gtcaagctct |
| | aaatcggggg ctccctttag ggttccgatt tagtgcttta cggcacctcg accccaaaaa |
| | acttgattag ggtgatggtt cacgtagtgg gccatcgccc tgatagacgg tttttcgccc |
| | tttgacgttg gagtccacgt tctttaatag tggactcttg ttccaaactg gaacaacact |
| | caaccctatc tcggtctatt cttttgattt ataagggatt ttgccgattt cggcctattg |
| | gttaaaaaat gagctgattt aacaaaaatt taacgcgaat tttaacaaaa tattaacgtt |
| | tacaatttaa atatttgctt atacaatctt cctgtttttg gggcttttct gattatcaac |
| | cggggtacat atgattgaca tgctagtttt acgattaccg ttcatcgatt tagggttagg |
| | cgttttgcgc tgcttcgcga tgtacgggcc agatatacgc gttgacattg attattgact |
| | agttattaat agtaatcaat tacggggtca ttagttcata gcccatatat ggagttccgc |
| | gttacataac ttacggtaaa tggcccgcct ggctgaccgc ccaacgaccc ccgcccattg |
| | acgtcaataa tgacgtatgt tcccatagta acgccaatag ggactttcca ttgacgtcaa |
| | tgggtggact atttacggta aactgcccac ttggcagtac atcaagtgta tcatatgcca |
| | agtacgcccc ctattgacgt caatgacggt aaatggcccg cctggcatta tgcccagtac |
| | atgaccttat gggactttcc tacttggcag tacatctacg tattagtcat cgctattacc |
| | atggtgatgc ggttttggca gtacatcaat gggcgtggat agcggtttga ctcacgggga |
| | tttccaagtc tccaccccat tgacgtcaat gggagtttgt tttggcacca aaatcaacgg |
| | gactttccaa aatgtcgtaa caactccgcc ccattgacgc aaatgggcgg taggcgtgta |
| | cggtggggta cttatataag ggggtggggg cgcgttcgtc ctcagtcgcg atcgaacact |
| | cgagccgagc agacgtgcct acggaccgta atacgactca ctatagggaa gcttggcatt |
| | ccggtactgt tggtaaagcc accatggctt ccaaggtgta cgaccccgag caacgcaaac |
| | gcatgatcac tgggcctcag tggtgggctc gctgcaagca aatgaacgtg ctggactcct |
| | tcatcaacta ctatgattcc gagaagcacg ccgagaacgc cgtgattttt ctgcatggta |
| | acgctgcctc cagctacctg tggaggcacg tcgtgcctca catcgagccc gtggctagat |
| | gcatcatccc tgatctgatc ggaatgggta agtccggcaa gagcgggaat ggctcatatc |
| | gcctcctgga tcactacaag tacctcaccg cttggttcga gctgctgaac cttccaaaga |
| | aaatcatctt tgtgggccac gactgggggg cttgtctggc ctttcactac tcctacgagc |
| | accaagacaa gatcaaggcc atcgtccatg ctgagagtgt cgtggacgtg atcgagtcct |
| | gggacgagtg gcctgacatc gaggaggata tcgccctgat caagagcgaa gagggcgaga |
| | aaatggtgct tgagaataac ttcttcgtcg agaccatgct cccaagcaag atcatgcgga |
| | aactggagcc tgaggagttc gctgcctacc tggagccatt caaggagaag ggcgaggtta |
| | gacggcctac cctctcctgg cctcgcgaga tccctctcgt taagggaggc aagcccgacg |
| | tcgtccagat tgtccgcaac tacaacgcct accttcgggc cagcgacgat ctgcctaaga |
| | tgttcatcga gtccgaccct gggttctttt ccaacgctat tgtcgaggga gctaagaagt |
| | tccctaacac cgagttcgtg aaggtgaagg gcctccactt cagccaggag gacgctccag |
| | atgaaatggg taagtacatc aagagcttcg tggagcgcgt gctgaagaac gagcagtaat |
| | tctagagggc cctattctat agtgtcacct aaatgctaga gctcgctgat cagcctcgac |
| | tgtgccttct agttgccagc catctgttgt ttgcccctcc cccgtgcctt ccttgaccct |
| | ggaaggtgcc actcccactg tcctttccta ataaaatgag gaaattgcat cgcattgtct |
| | gagtaggtgt cattctattc tggggggtgg ggtggggcag gacagcaagg gggaggattg |
| | ggaagacaat agcaggcatg ctggggatgc ggtgggctct atggcttctg aggcggaaag |
| | aaccagctgg ggctcgaggg ggatcgattc tcttgtttgc tccagactct caggcaatga |
| | cctgatagcc tttgtagaga cctctcaaaa atagctaccc tctccggcat gaatttatca |
| | gctagaacgg ttgaatatca tattgatggt gatttgactg tctccggcct ttctcacccg |
| | tttgaatctt tacctacaca ttactcaggc attgcattta aaatatatga gggttctaaa |
| | aatttttatc cttgcgttga aataaaggct tctcccgcaa aagtattaca gggtcataat |
| | gtttttggta caaccgattt agctttatgc tctgaggctt tattgcttaa ttttgctaat |
| | tctttgcctt gcctgtatga tttattggat gttggaattc ctgatgcggt attttctcct |
| | tacgcatctg tgcggtattt cacaccgcat atggtgcact ctcagtacaa tctgctctga |
| | tgccgcatag ttaagccagc cccgacaccc gccaacaccc gctgacgcgc cctgacgggc |
| | ttgtctgctc ccggcatccg cttacagaca agctgtgacc gtctccggga gctgcatgtg |
| | tcagaggttt tcaccgtcat caccgaaacg cgcgagacga aagggcctcg tgatacgcct |
| | atttttatag gttaatgtca tgataataat ggtttcttag acgtcaggtg gcacttttcg |
| | gggaaatgtg cgcggaaccc ctatttgttt atttttctaa atacattcaa atatgtatcc |
| | gctcatgaga caataaccct gataaatgct tcaataatat tgaaaaagga agagtatgag |
| | tattcaacat ttccgtgtcg cccttattcc cttttttgcg gcattttgcc ttcctgtttt |
| | tgctcaccca gaaacgctgg tgaaagtaaa agatgctgaa gatcagttgg gtgcacgagt |
| | gggttacatc gaactggatc tcaacagcgg taagatcctt gagagttttc gccccgaaga |
| | acgttttcca atgatgagca cttttaaagt tctgctatgt ggcgcggtat tatcccgtat |
| | tgacgccggg caagagcaac tcggtcgccg catacactat tctcagaatg acttggttga |
| | gtactcacca gtcacagaaa agcatcttac ggatggcatg acagtaagag aattatgcag |
| | tgctgccata accatgagtg ataacactgc ggccaactta cttctgacaa cgatcggagg |
| | accgaaggag ctaaccgctt ttttgcacaa catgggggat catgtaactc gccttgatcg |
| | ttgggaaccg gagctgaatg aagccatacc aaacgacgag cgtgacacca cgatgcctgt |
| | agcaatggca acaacgttgc gcaaactatt aactggcgaa ctacttactc tagcttcccg |
| | gcaacaatta atagactgga tggaggcgga taaagttgca ggaccacttc tgcgctcggc |
| | ccttccggct ggctggttta ttgctgataa atctggagcc ggtgagcgtg ggtctcgcgg |
| | tatcattgca gcactggggc cagatggtaa gccctcccgt atcgtagtta tctacacgac |
| | ggggagtcag gcaactatgg atgaacgaaa tagacagatc gctgagatag gtgcctcact |
| | gattaagcat tggtaactgt cagaccaagt ttactcatat atactttaga ttgatttaaa |
| | acttcatttt taatttaaaa ggatctaggt gaagatcctt tttgataatc tcatgaccaa |
| | aatcccttaa cgtgagtttt cgttccactg agcgtcagac cccgtagaaa agatcaaagg |
| | atcttcttga gatccttttt ttctgcgcgt aatctgctgc ttgcaaacaa aaaaaccacc |
| | gctaccagcg gtggtttgtt tgccggatca agagctacca actctttttc cgaaggtaac |
| | tggcttcagc agagcgcaga taccaaatac tgtccttcta gtgtagccgt agttaggcca |
| | ccacttcaag aactctgtag caccgcctac atacctcgct ctgctaatcc tgttaccagt |
| | ggctgctgcc agtggcgata agtcgtgtct taccgggttg gactcaagac gatagttacc |
| | ggataaggcg cagcggtcgg gctgaacggg gggttcgtgc acacagccca gcttggagcg |
| | aacgacctac accgaactga gatacctaca gcgtgagcta tgagaaageg ccacgcttcc |
| | cgaagggaga aaggcggaca ggtatccggt aagcggcagg gtcggaacag gagagcgcac |
| | gagggagctt ccagggggaa acgcctggta tctttatagt cctgtegggt ttegccacct |
| | ctgacttgag cgtcgatttt tgtgatgctc gtcagggggg cggagcctat ggaaaaacgc |
| | cagcaacgeg gcctttttac ggttcctggc cttttgctgg ccttttgctc acatgttctt |
| | tcctgegtta tcccctgatt ctgtggataa ccgtattacc gcctttgagt gagctgatac |
| | cgctcgccgc agccgaacga ccgagcgcag cgagtcagtg agcgaggaag cggaagagcg |
| | cccaatacge aaacegccte tccccgegcg ttggccgatt cattaatg |
| SEQ ID NO: 5 | cagctgcgcgctcgctcgctcactgaggccgcccgggcaaagcccgggcgtcgggcgacctttggtcg |
| pAAV-CK8- | cccggcctcagtgagcgagcgagcgcgcagagagggagtggccaactccatcactaggggttccttgt |
| int-MBNL1- | agttaatgattaacccgccatgctacttatctacaaactctagagcggccgcacgcgtacgctagctagact |
| DMPKmir97- | agcatgctgcccatgtaaggaggcaaggcctggggacacccgagatgcctggttataattaacccagac |
| IRES- | atgtggctgcccccccccccccaacacctgctgcctctaaaaataaccctgcatgccatgttcccggcgaa |
| mir208aTS | gggccagctgtcccccgccagctagactcagcacttagtttaggaaccagtgagcaagtcagcccttggg |
| (intron | gcagcccatacaaggccatggggctgggcaagctgcacgcctgggtccggggtgggcacggtgcccg |
| bolded, | ggcaacgagctgaaagctcatctgctctcaggggcccctccctggggacagcccctcctggctagtcaca |
| MBNL1 | ccctgtaggctcctctatataacccaggggcacaggggctgccctcattctaccaccacctccacagcaca |
| inclusive of | gacagacactcaggagccagccagcgtatacgtacttctgacacaacagtctcgaacttaagctgcag |
| stop codon | aagttggtcgtgaggcactgggcaggtaagtatcaaggttacaagacaggtttaaacggagacca |
| underlined, | atagaaactgggcttgtcgagacagagaagactcttgcgtttctgataggcacctattggtcttactg |
| DMPK | acatccactttgcctttctctccacaggtgtccactcccagttcaattacagctcttaaggctagagta |
| mir97 bolded | accggtgccaccATGGCAGTTAGTGTTACTCCGATACGCGATACCAAG |
| italics, and | TGGCTCACTCTCGAAGTCTGTAGAGAATTTCAGCGAGGGACCTG |
| miR208aTSx3 | CTCCCGACCTGATACTGAATGTAAGTTTGCGCACCCATCTAAAT |
| in | CATGTCAAGTGGAGAACGGCAGGGTGATTGCATGTTTCGACAG |
| underlined | TCTCAAAGGCCGGTGTTCACGAGAGAATTGCAAGTATCTTCATC |
| italics) | CACCACCGCACCTCAAGACTCAGCTTGAAATAAACGGCCGCAA |
| | TAACCTTATACAACAGAAGAACATGGCCATGCTGGCACAACAA |
| | ATGCAATTGGCCAACGCAATGATGCCAGGGGCGCCCCTGCAAC |
| | CAGTTCCAATGTTCTCAGTTGCACCATCACTCGCAACAAATGCA |
| | TCCGCTGCTGCCTTCAACCCGTACCTTGGGCCCGTTTCACCCAG |
| | CCTTGTGCCCGCTGAGATTCTCCCTACAGCGCCTATGCTGGTAA |
| | CGGGTAACCCTGGTGTCCCCGTTCCCGCAGCAGCGGCCGCAGCC |
| | GCTCAGAAATTGATGCGCACCGACAGATTGGAGGTATGCCGAG |
| | AGTACCAGCGCGGAAACTGTAATAGAGGGGAAAATGACTGCCG |
| | GTTTGCCCACCCAGCTGACTCAACAATGATTGACACTAATGACA |
| | ATACGGTTACCGTTTGTATGGACTACATAAAAGGACGGTGCTCC |
| | CGCGAAAAATGCAAATACTTCCATCCCCCAGCGCATCTTCAGGC |
| | CAAGATTAAGGCAGCCCAATACCAAGTCAACCAGGCAGCGGCT |
| | GCGCAAGCAGCAGCCACGGCAGCAGCAATGGGCATTCCGCAGG |
| | CAGTACTCCCACCCCTTCCAAAGAGGCCGGCGCTCGAGAAGAC |
| | CAATGGTGCAACAGCTGTGTTTAACACGGGTATCTTTCAATATC |
| | AGCAAGCACTTGCGAACATGCAACTCCAACAACATACTGCGTTT |
| | TTGCCACCAGTCCCAATGGTACATGGCGCTACACCGGCGACAGT |
| | AAGTGCTGCGACAACGAGCGCTACATCCGTGCCATTCGCTGCGA |
| | CAGCTACAGCGAACCAGATCCCGATCATCTCCGCAGAGCACTTG |
| | ACTTCCCATAAGTACGTTACTCAGATGTAGtccggagtttcgagtgagcgcg |
| | gcttaaggaggtccgaTtgactgtaaagccacagatgctTagtcggacctccttaagccacgcctact |
| | agaaccctaggGGCGCGcCCGCCCCTCTCCCTCCCCCCCCCCTAACGT |
| | TACTGGCCGAAGCCGCTTGGAATAAGGCCGGTGTGCGTTTGTCT |
| | ATATGTTATTTTCCACCATATTGCCGTCTTTTGGCAATGTGAGGG |
| | CCCGGAAACCTGGCCCTGTCTTCTTGACGAGCATTCCTAGGGGT |
| | CTTTCCCCTCTCGCCAAAGGAATGCAAGGTCTGTTGAATGTCGT |
| | GAAGGAAGCAGTTCCTCTGGAAGCTTCTTGAAGACAAACAACG |
| | TCTGTAGCGACCCTTTGCAGGCAGCGGAACCCCCCACCTGGCGA |
| | CAGGTGCCTCTGCGGCCAAAAGCCACGTGTATAAGATACACCT |
| | GCAAAGGCGGCACAACCCCAGTGCCACGTTGTGAGTTGGATAG |
| | TTGTGGAAAGAGTCAAATGGCTCTCCTCAAGCGTATTCAACAAG |
| | GGGCTGAAGGATGCCCAGAAGGTACCCCATTGTATGGGATCTG |
| | ATCTGGGGCCTCGGTGCACATGCTTTACATGTGTTTAGTCGAGG |
| | TTAAAAAAACGTCTAGGCCCCCCGAACCACGGGGACGTGGTTTT |
| | CCTTTGAAAAACACGATGATAAtTGTACACCTGCAGGCACatcgGT |
| | GGACctgaGTCACAAGCTTTTTGCTCGTCTTATGTCGACCCTCGAGGA |
| | CAAGCTTTTTGCTCGTCTTATGTCGACGACctgaGTCACAAGCTTTTTG |
| | CTCGTCTTATCACatcgGTGCCTGCAGGTGTACAagggAACTTGTTTA |
| | TTGCAGCTTATAATGGTTACAAATAAAGCAATAGCATCACAAAT |
| | TTCACAAATAAAGCATTTTTTTCACTGCagatctgaattggccgtttgtagataag |
| | tagcatggcgggttaatcattaactacaaggaacccctagtgatggagttggccactccctctctgcgcgct |
| | cgctcgctcactgaggccgggcgaccaaaggtcgcccgacgcccgggctttgcccgggcggcctcagt |
| | gagcgagcgagcgcgcagctggcgtaatagcgaagaggcccgcaccgatcgcccttcccaacagttgc |
| | gcagcctgaatggcgaatggcgattccgttgcaatggctggcggtaatattgttctggatattaccagcaag |
| | gccgatagtttgagttcttctactcaggcaagtgatgttattactaatcaaagaagtattgcgacaacggttaa |
| | tttgcgtgatggacagactcttttactcggtggcctcactgattataaaaacacttctcaggattctggcgtac |
| | cgttcctgtctaaaatccctttaatcggcctcctgtttagctcccgctctgattctaacgaggaaagcacgttat |
| | acgtgctcgtcaaagcaaccatagtacgcgccctgtagcggcgcattaagcgcggcgggtgtggtggtta |
| | cgcgcagcgtgaccgctacacttgccagcgccctagcgcccgctcctttcgctttcttcccttcctttctcgc |
| | cacgttcgccggctttccccgtcaagctctaaatcgggggctccctttagggttccgatttagtgctttacgg |
| | cacctcgaccccaaaaaacttgattagggtgatggttcacgtagtgggccatcgccctgatagacggttttt |
| | cgccctttgacgttggagtccacgttctttaatagtggactcttgttccaaactggaacaacactcaaccctat |
| | ctcggtctattcttttgatttataagggattttgccgatttcggcctattggttaaaaaatgagctgatttaacaaa |
| | aatttaacgcgaattttaacaaaatattaacgtttacaatttaaatatttgcttatacaatcttcctgtttttggggc |
| | ttttctgattatcaaccggggtacatatgattgacatgctagttttacgattaccgttcatCGATTTAGG |
| | GTTAGGCGTTTTGCGCTGCTTCGCGATGTACGGGCCAGATATAcg |
| | cgttgacattgattattgactagttattaatagtaatcaattacggggtcattagttcatagcccatatatggagt |
| | tccgcgttacataacttacggtaaatggcccgcctggctgaccgcccaacgacccccgcccattgacgtc |
| | aataatgacgtatgttcccatagtaacgccaatagggactttccattgacgtcaatgggtggactatttacgg |
| | taaactgcccacttggcagtacatcaagtgtatcatatgccaagtacgccccctattgacgtcaatgacggt |
| | aaatggcccgcctggcattatgcccagtacatgaccttatgggactttcctacttggcagtacatctacgtatt |
| | agtcatcgctattaccatggtgatgcggttttggcagtacatcaatgggcgtggatagcggtttgactcacg |
| | gggatttccaagtctccaccccattgacgtcaatgggagtttgttttggcaccaaaatcaacgggactttcca |
| | aaatgtcgtaacaactccgccccattgacgcaaatgggcggtaggcgtgtacggtggggtacttatataag |
| | ggggtgggggcgcgttcgtcctcagtcgcgatcgaacactcgagccgagcagacgtgcctacggaccg |
| | taatacgactcactatagggaagcttggcattccggtactgttggtaaagccaccatggcttccaaggtgta |
| | cgaccccgagcaacgcaaacgcatgatcactgggcctcagtggtgggctcgctgcaagcaaatgaacgt |
| | gctggactccttcatcaactactatgattccgagaagcacgccgagaacgccgtgatttttctgcatggtaac |
| | gctgcctccagctacctgtggaggcacgtcgtgcctcacatcgagcccgtggctagatgcatcatccctga |
| | tctgatcggaatgggtaagtccggcaagagcgggaatggctcatatcgcctcctggatcactacaagtac |
| | ctcaccgcttggttcgagctgctgaaccttccaaagaaaatcatctttgtgggccacgactggggggcttgt |
| | ctggcctttcactactcctacgagcaccaagacaagatcaaggccatcgtccatgctgagagtgtcgtgga |
| | cgtgatcgagtcctgggacgagtggcctgacatcgaggaggatatcgccctgatcaagagcgaagagg |
| | gcgagaaaatggtgcttgagaataacttcttcgtcgagaccatgctcccaagcaagatcatgcggaaactg |
| | gagcctgaggagttcgctgcctacctggagccattcaaggagaagggcgaggttagacggcctaccctc |
| | tcctggcctcgcgagatccctctcgttaagggaggcaagcccgacgtcgtccagattgtccgcaactaca |
| | acgcctaccttcgggccagcgacgatctgcctaagatgttcatcgagtccgaccctgggttcttttccaacg |
| | ctattgtcgagggagctaagaagttccctaacaccgagttcgtgaaggtgaagggcctccacttcagcca |
| | ggaggacgctccagatgaaatgggtaagtacatcaagagcttcgtggagcgcgtgctgaagaacgagc |
| | agtaattctagagggccctattctatagtgtcacctaaatgctagagctcgctgatcagcctcgactgtgcctt |
| | ctagttgccagccatctgttgtttgcccctcccccgtgccttccttgaccctggaaggtgccactcccactgt |
| | cctttcctaataaaatgaggaaattgcatcgcattgtctgagtaggtgtcattctattctggggggtggggtg |
| | gggcaggacagcaagggggaggattgggaagacaatagcaggcatgctggggatgcggtgGGCT |
| | CTATGGCTTCTGAGGCGGAAAGAACCAGCTGGGGCTCGAGGGG |
| | GATcgattctcttgtttgctccagactctcaggcaatgacctgatagcctttgtagagacctctcaaaaata |
| | gctaccctctccggcatgaatttatcagctagaacggttgaatatcatattgatggtgatttgactgtctccgg |
| | cctttctcacccgtttgaatctttacctacacattactcaggcattgcatttaaaatatatgagggttctaaaaatt |
| | tttatccttgcgttgaaataaaggcttctcccgcaaaagtattacagggtcataatgtttttggtacaaccgattt |
| | agctttatgctctgaggctttattgcttaattttgctaattctttgccttgcctgtatgatttattggatgttggaat |
| | tcctgatgcggtattttctccttacgcatctgtgcggtatttcacaccgcatatggtgcactctcagtacaatctgc |
| | tctgatgccgcatagttaagccagccccgacacccgccaacacccgctgacgcgccctgacgggcttgt |
| | ctgctcccggcatccgcttacagacaagctgtgaccgtctccgggagctgcatgtgtcagaggttttcacc |
| | gtcatcaccgaaacgcgcgagacgaaagggcctcgtgatacgcctatttttataggttaatgtcatgataat |
| | aatggtttcttagacgtcaggtggcacttttcggggaaatgtgcgcggaacccctatttgtttatttttctaaata |
| | cattcaaatatgtatccgctcatgagacaataaccctgataaatgcttcaataatattgaaaaaggaagagtat |
| | gagtattcaacatttccgtgtcgcccttattcccttttttgcggcattttgccttcctgtttttgctcacccagaaa |
| | cgctggtgaaagtaaaagatgctgaagatcagttgggtgcacgagtgggttacatcgaactggatctcaac |
| | agcggtaagatccttgagagttttcgccccgaagaacgttttccaatgatgagcacttttaaagttctgctatg |
| | tggcgcggtattatcccgtattgacgccgggcaagagcaactcggtcgccgcatacactattctcagaatg |
| | acttggttgagtactcaccagtcacagaaaagcatcttacggatggcatgacagtaagagaattatgcagtg |
| | ctgccataaccatgagtgataacactgcggccaacttacttctgacaacgatcggaggaccgaaggagct |
| | aaccgcttttttgcacaacatgggggatcatgtaactcgccttgatcgttgggaaccggagctgaatgaagc |
| | cataccaaacgacgagcgtgacaccacgatgcctgtagcaatggcaacaacgttgcgcaaactattaact |
| | ggcgaactacttactctagcttcccggcaacaattaatagactggatggaggcggataaagttgcaggacc |
| | acttctgcgctcggcccttccggctggctggtttattgctgataaatctggagccggtgagcgtgggtctcg |
| | cggtatcattgcagcactggggccagatggtaagccctcccgtatcgtagttatctacacgacggggagtc |
| | aggcaactatggatgaacgaaatagacagatcgctgagataggtgcctcactgattaagcattggtaactg |
| | tcagaccaagtttactcatatatactttagattgatttaaaacttcatttttaatttaaaaggatctaggtgaagat |
| | cctttttgataatctcatgaccaaaatcccttaacgtgagttttcgttccactgagcgtcagaccccgtagaaa |
| | agatcaaaggatcttcttgagatcctttttttctgcgcgtaatctgctgcttgcaaacaaaaaaaccaccgcta |
| | ccagcggtggtttgtttgccggatcaagagctaccaactctttttccgaaggtaactggcttcagcagagcg |
| | cagataccaaatactgtccttctagtgtagccgtagttaggccaccacttcaagaactctgtagcaccgccta |
| | catacctcgctctgctaatcctgttaccagtggctgctgccagtggcgataagtcgtgtcttaccgggttgga |
| | ctcaagacgatagttaccggataaggcgcagcggtcgggctgaacggggggttcgtgcacacagccca |
| | gcttggagcgaacgacctacaccgaactgagatacctacagcgtgagctatgagaaagcgccacgcttc |
| | ccgaagggagaaaggcggacaggtatccggtaagcggcagggtcggaacaggagagcgcacgagg |
| | gagcttccagggggaaacgcctggtatctttatagtcctgtcgggtttcgccacctctgacttgagcgtcgat |
| | ttttgtgatgctcgtcaggggggcggagcctatggaaaaacgccagcaacgcggcctttttacggttcctg |
| | gccttttgctggccttttgctcacatgttctttcctgcgttatcccctgattctgtggataaccgtattaccgccttt |
| | gagtgagctgataccgctcgccgcagccgaacgaccgagcgcagcgagtcagtgagcgaggaagcg |
| | gaagagcgcccaatacgcaaaccgcctctccccgcgcgttggccgattcattaatg |
| SEQ ID NO: | cagctgcgcgctcgctcgctcactgaggccgcccgggcaaagcccgggcgtcgggcgacctttggtcg |
| 6 | cccggcctcagtgagcgagcgagcgcgcagagagggagtggccaactccatcactaggggttccttgt |
| pAA | agttaatgattaacccgccatgctacttatctacaaactctagagcggccgcacgcgtacgctagctagact |
| V-Ck8- | agcatgctgcccatgtaaggaggcaaggcctggggacacccgagatgcctggttataattaacccagac |
| nointron- | atgtggctgcccccccccccccaacacctgctgcctctaaaaataaccctgcatgccatgttcccggcgaa |
| mbnl1- | gggccagctgtcccccgccagctagactcagcacttagtttaggaaccagtgagcaagtcagcccttggg |
| RESBsp1407i_ | gcagcccatacaaggccatggggctgggcaagctgcacgcctgggtccggggtgggcacggtgcccg |
| mir208a3XTS- | ggcaacgagctgaaagctcatctgctctcaggggcccctccctggggacagcccctcctggctagtcaca |
| CMVRenilla_ | ccctgtaggctcctctatataacccaggggcacaggggctgccctcattctaccaccacctccacagcaca |
| PARENT | gacagacactcaggagccagccagcgtaccggtgccaccATGGCAGTTAGTGTTACT |
| (MBNL1 | CCGATACGCGATACCAAGTGGCTCACTCTCGAAGTCTGTAGAGA |
| underlined, | ATTTCAGCGAGGGACCTGCTCCCGACCTGATACTGAATGTAAGT |
| and | TTGCGCACCCATCTAAATCATGTCAAGTGGAGAACGGCAGGGT |
| miR208aTSx3 | GATTGCATGTTTCGACAGTCTCAAAGGCCGGTGTTCACGAGAGA |
| in italics) | ATTGCAAGTATCTTCATCCACCACCGCACCTCAAGACTCAGCTT |
| | GAAATAAACGGCCGCAATAACCTTATACAACAGAAGAACATGG |
| | CCATGCTGGCACAACAAATGCAATTGGCCAACGCAATGATGCC |
| | AGGGGCGCCCCTGCAACCAGTTCCAATGTTCTCAGTTGCACCAT |
| | CACTCGCAACAAATGCATCCGCTGCTGCCTTCAACCCGTACCTT |
| | GGGCCCGTTTCACCCAGCCTTGTGCCCGCTGAGATTCTCCCTAC |
| | AGCGCCTATGCTGGTAACGGGTAACCCTGGTGTCCCCGTTCCCG |
| | CAGCAGCGGCCGCAGCCGCTCAGAAATTGATGCGCACCGACAG |
| | ATTGGAGGTATGCCGAGAGTACCAGCGCGGAAACTGTAATAGA |
| | GGGGAAAATGACTGCCGGTTTGCCCACCCAGCTGACTCAACAA |
| | TGATTGACACTAATGACAATACGGTTACCGTTTGTATGGACTAC |
| | ATAAAAGGACGGTGCTCCCGCGAAAAATGCAAATACTTCCATC |
| | CCCCAGCGCATCTTCAGGCCAAGATTAAGGCAGCCCAATACCA |
| | AGTCAACCAGGCAGCGGCTGCGCAAGCAGCAGCCACGGCAGCA |
| | GCAATGGGCATTCCGCAGGCAGTACTCCCACCCCTTCCAAAGAG |
| | GCCGGCGCTCGAGAAGACCAATGGTGCAACAGCTGTGTTTAAC |
| | ACGGGTATCTTTCAATATCAGCAAGCACTTGCGAACATGCAACT |
| | CCAACAACATACTGCGTTTTTGCCACCAGTCCCAATGGTACATG |
| | GCGCTACACCGGCGACAGTAAGTGCTGCGACAACGAGCGCTAC |
| | ATCCGTGCCATTCGCTGCGACAGCTACAGCGAACCAGATCCCGA |
| | TCATCTCCGCAGAGCACTTGACTTCCCATAAGTACGTTACTCAG |
| | ATGTAGtccggagttaaccctaggGGCGCGcCCGCCCCTCTCCCTCCCCCC |
| | CCCCTAACGTTACTGGCCGAAGCCGCTTGGAATAAGGCCGGTGT |
| | GCGTTTGTCTATATGTTATTTTCCACCATATTGCCGTCTTTTGGC |
| | AATGTGAGGGCCCGGAAACCTGGCCCTGTCTTCTTGACGAGCAT |
| | TCCTAGGGGTCTTTCCCCTCTCGCCAAAGGAATGCAAGGTCTGT |
| | TGAATGTCGTGAAGGAAGCAGTTCCTCTGGAAGCTTCTTGAAGA |
| | CAAACAACGTCTGTAGCGACCCTTTGCAGGCAGCGGAACCCCC |
| | CACCTGGCGACAGGTGCCTCTGCGGCCAAAAGCCACGTGTATA |
| | AGATACACCTGCAAAGGCGGCACAACCCCAGTGCCACGTTGTG |
| | AGTTGGATAGTTGTGGAAAGAGTCAAATGGCTCTCCTCAAGCGT |
| | ATTCAACAAGGGGCTGAAGGATGCCCAGAAGGTACCCCATTGT |
| | ATGGGATCTGATCTGGGGCCTCGGTGCACATGCTTTACATGTGT |
| | TTAGTCGAGGTTAAAAAAACGTCTAGGCCCCCCGAACCACGGG |
| | GACGTGGTTTTCCTTTGAAAAACACGATGATAAtTGTACACCTGC |
| | AGGCACatcgGTGGACctgaGTCACAAGCTTTTTGCTCGTCTTATGTCG |
| | ACCCTCGAGGACAAGCTTTTTGCTCGTCTTATGTCGACGACctgaGTC |
| | ACAAGCTTTTTGCTCGTCTTATCACatcgGTGCCTGCAGGTGTACAag |
| | ggAACTTGTTTATTGCAGCTTATAATGGTTACAAATAAAGCAATA |
| | GCATCACAAATTTCACAAATAAAGCATTTTTTTCACTGCagatctgaa |
| | ttggccgtttgtagataagtagcatggcgggttaatcattaactacaaggaacccctagtgatggagttggc |
| | cactccctctctgcgcgctcgctcgctcactgaggccgggcgaccaaaggtcgcccgacgcccgggctt |
| | tgcccgggcggcctcagtgagcgagcgagcgcgcagctggcgtaatagcgaagaggcccgcaccgat |
| | cgcccttcccaacagttgcgcagcctgaatggcgaatggcgattccgttgcaatggctggcggtaatattg |
| | ttctggatattaccagcaaggccgatagtttgagttcttctactcaggcaagtgatgttattactaatcaaaga |
| | agtattgcgacaacggttaatttgcgtgatggacagactcttttactcggtggcctcactgattataaaaacac |
| | ttctcaggattctggcgtaccgttcctgtctaaaatccctttaatcggcctcctgtttagctcccgctctgattct |
| | aacgaggaaagcacgttatacgtgctcgtcaaagcaaccatagtacgcgccctgtagcggcgcattaagc |
| | gcggcgggtgtggtggttacgcgcagcgtgaccgctacacttgccagcgccctagcgcccgctcctttcg |
| | ctttcttcccttcctttctcgccacgttcgccggctttccccgtcaagctctaaatcgggggctccctttagggt |
| | tccgatttagtgctttacggcacctcgaccccaaaaaacttgattagggtgatggttcacgtagtgggccatc |
| | gccctgatagacggtttttcgccctttgacgttggagtccacgttctttaatagtggactcttgttccaaactgg |
| | aacaacactcaaccctatctcggtctattcttttgatttataagggattttgccgatttcggcctattggttaaaa |
| | aatgagctgatttaacaaaaatttaacgcgaattttaacaaaatattaacgtttacaatttaaatatttgcttatac |
| | aatcttcctgtttttggggcttttctgattatcaaccggggtacatatgattgacatgctagttttacgattaccgt |
| | tcatCGATTTAGGGTTAGGCGTTTTGCGCTGCTTCGCGATGTACGG |
| | GCCAGATATAcgcgttgacattgattattgactagttattaatagtaatcaattacggggtcattagtt |
| | catagcccatatatggagttccgcgttacataacttacggtaaatggcccgcctggctgaccgcccaacga |
| | cccccgcccattgacgtcaataatgacgtatgttcccatagtaacgccaatagggactttccattgacgtcaa |
| | tgggtggactatttacggtaaactgcccacttggcagtacatcaagtgtatcatatgccaagtacgcccccta |
| | ttgacgtcaatgacggtaaatggcccgcctggcattatgcccagtacatgaccttatgggactttcctacttg |
| | gcagtacatctacgtattagtcatcgctattaccatggtgatgcggttttggcagtacatcaatgggcgtgga |
| | tagcggtttgactcacggggatttccaagtctccaccccattgacgtcaatgggagtttgttttggcaccaaa |
| | atcaacgggactttccaaaatgtcgtaacaactccgccccattgacgcaaatgggcggtaggcgtgtacg |
| | gtggggtacttatataagggggtgggggcgcgttcgtcctcagtcgcgatcgaacactcgagccgagca |
| | gacgtgcctacggaccgtaatacgactcactatagggaagcttggcattccggtactgttggtaaagccac |
| | catggcttccaaggtgtacgaccccgagcaacgcaaacgcatgatcactgggcctcagtggtgggctcg |
| | ctgcaagcaaatgaacgtgctggactccttcatcaactactatgattccgagaagcacgccgagaacgcc |
| | gtgatttttctgcatggtaacgctgcctccagctacctgtggaggcacgtcgtgcctcacatcgagcccgtg |
| | gctagatgcatcatccctgatctgatcggaatgggtaagtccggcaagagcgggaatggctcatatcgcct |
| | cctggatcactacaagtacctcaccgcttggttcgagctgctgaaccttccaaagaaaatcatctttgtgggc |
| | cacgactggggggcttgtctggcctttcactactcctacgagcaccaagacaagatcaaggccatcgtcca |
| | tgctgagagtgtcgtggacgtgatcgagtcctgggacgagtggcctgacatcgaggaggatatcgccctg |
| | atcaagagcgaagagggcgagaaaatggtgcttgagaataacttcttcgtcgagaccatgctcccaagca |
| | agatcatgcggaaactggagcctgaggagttcgctgcctacctggagccattcaaggagaagggcgag |
| | gttagacggcctaccctctcctggcctcgcgagatccctctcgttaagggaggcaagcccgacgtcgtcc |
| | agattgtccgcaactacaacgcctaccttcgggccagcgacgatctgcctaagatgttcatcgagtccgac |
| | cctgggttcttttccaacgctattgtcgagggagctaagaagttccctaacaccgagttcgtgaaggtgaag |
| | ggcctccacttcagccaggaggacgctccagatgaaatgggtaagtacatcaagagcttcgtggagcgc |
| | gtgctgaagaacgagcagtaattctagagggccctattctatagtgtcacctaaatgctagagctcgctgat |
| | cagcctcgactgtgccttctagttgccagccatctgttgtttgcccctcccccgtgccttccttgaccctggaa |
| | ggtgccactcccactgtcctttcctaataaaatgaggaaattgcatcgcattgtctgagtaggtgtcattctatt |
| | ctggggggtggggtggggcaggacagcaagggggaggattgggaagacaatagcaggcatgctggg |
| | gatgcggtgGGCTCTATGGCTTCTGAGGCGGAAAGAACCAGCTGGG |
| | GCTCGAGGGGGATcgattctcttgtttgctccagactctcaggcaatgacctgatagcctttgta |
| | gagacctctcaaaaatagctaccctctccggcatgaatttatcagctagaacggttgaatatcatattgatggt |
| | gatttgactgtctccggcctttctcacccgtttgaatctttacctacacattactcaggcattgcatttaaaatata |
| | tgagggttctaaaaatttttatccttgcgttgaaataaaggcttctcccgcaaaagtattacagggtcataatgt |
| | ttttggtacaaccgatttagctttatgctctgaggctttattgcttaattttgctaattctttgccttgcctgtatga |
| | tttattggatgttggaattcctgatgcggtattttctccttacgcatctgtgcggtatttcacaccgcatatggtgca |
| | ctctcagtacaatctgctctgatgccgcatagttaagccagccccgacacccgccaacacccgctgacgc |
| | gccctgacgggcttgtctgctcccggcatccgcttacagacaagctgtgaccgtctccgggagctgcatgt |
| | gtcagaggttttcaccgtcatcaccgaaacgcgcgagacgaaagggcctcgtgatacgcctatttttatag |
| | gttaatgtcatgataataatggtttcttagacgtcaggtggcacttttcggggaaatgtgcgcggaaccccta |
| | tttgtttatttttctaaatacattcaaatatgtatccgctcatgagacaataaccctgataaatgcttcaataatatt |
| | gaaaaaggaagagtatgagtattcaacatttccgtgtcgcccttattcccttttttgcggcattttgccttcctgt |
| | ttttgctcacccagaaacgctggtgaaagtaaaagatgctgaagatcagttgggtgcacgagtgggttaca |
| | tcgaactggatctcaacagcggtaagatccttgagagttttcgccccgaagaacgttttccaatgatgagca |
| | cttttaaagttctgctatgtggcgcggtattatcccgtattgacgccgggcaagagcaactcggtcgccgca |
| | tacactattctcagaatgacttggttgagtactcaccagtcacagaaaagcatcttacggatggcatgacagt |
| | aagagaattatgcagtgctgccataaccatgagtgataacactgcggccaacttacttctgacaacgatcg |
| | gaggaccgaaggagctaaccgcttttttgcacaacatgggggatcatgtaactcgccttgatcgttgggaa |
| | ccggagctgaatgaagccataccaaacgacgagcgtgacaccacgatgcctgtagcaatggcaacaac |
| | gttgcgcaaactattaactggcgaactacttactctagcttcccggcaacaattaatagactggatggaggc |
| | ggataaagttgcaggaccacttctgcgctcggcccttccggctggctggtttattgctgataaatctggagc |
| | cggtgagcgtgggtctcgcggtatcattgcagcactggggccagatggtaagccctcccgtatcgtagtta |
| | tctacacgacggggagtcaggcaactatggatgaacgaaatagacagatcgctgagataggtgcctcact |
| | gattaagcattggtaactgtcagaccaagtttactcatatatactttagattgatttaaaacttcatttttaatttaa |
| | aaggatctaggtgaagatcctttttgataatctcatgaccaaaatcccttaacgtgagttttcgttccactgagc |
| | gtcagaccccgtagaaaagatcaaaggatcttcttgagatcctttttttctgcgcgtaatctgctgcttgcaaa |
| | caaaaaaaccaccgctaccagcggtggtttgtttgccggatcaagagctaccaactctttttccgaaggtaa |
| | ctggcttcagcagagcgcagataccaaatactgtccttctagtgtagccgtagttaggccaccacttcaaga |
| | actctgtagcaccgcctacatacctcgctctgctaatcctgttaccagtggctgctgccagtggcgataagtc |
| | gtgtcttaccgggttggactcaagacgatagttaccggataaggcgcagcggtcgggctgaacgggggg |
| | ttcgtgcacacagcccagcttggagcgaacgacctacaccgaactgagatacctacagcgtgagctatga |
| | gaaagcgccacgcttcccgaagggagaaaggcggacaggtatccggtaagcggcagggtcggaaca |
| | ggagagcgcacgagggagcttccagggggaaacgcctggtatctttatagtcctgtcgggtttcgccacc |
| | tctgacttgagcgtcgatttttgtgatgctcgtcaggggggcggagcctatggaaaaacgccagcaacgc |
| | ggcctttttacggttcctggccttttgctggccttttgctcacatgttctttcctgcgttatcccctgattctgtgga |
| | taaccgtattaccgcctttgagtgagctgataccgctcgccgcagccgaacgaccgagcgcagcgagtca |
| | gtgagcgaggaagcggaagagcgcccaatacgcaaaccgcctctccccgcgcgttggccgattcattaa |
| | tg |

The first identification of muscleblind protein was in Drosophila. It was shown to be an RNA binding protein that acts as a required regulatory factor for differentiation of photoreceptor cells and muscle Z-bands. This factor binds to pre-mRNA in a sequence specific fashion at the common YGCY motif in pre-mRNAs and mRNAs, thereby modulating alternative splicing. In mammals there are 3 homologues of mbl: MBNL1 (HGNC: 6923 NCBI Entrez Gene: 4154 Ensembl: ENSG00000152601 OMIM®: 606516 UniProtKB/Swiss-Prot: Q9NR56), MBNL2, and MBNL3; each of which produce many alternatively spliced transcripts. MBNL proteins bind and localize with expanded double-stranded CUG RNA, but not normal length CUG repeats, in DM1 cells. Transgenic mouse knockout (KO) models of MBLN1 demonstrate that MBNL1 loss in mice causes many DM features, such as myotonia, abnormal myofibers, cataracts, and alterations in normal adult splicing patterns of mRNAs. MBNL2 can partially compensate for loss of MNBL1 in skeletal muscle and heart, but contributes to brain functional defects in mice, similar to DM. A transgenic knockout of MBNL2 and has recently been shown to protect brain structural integrity with MBNL1. MBNL3 KO transgenic mice displayed an age-associated decline in skeletal muscle regeneration.

Compound loss of MBNL1 and two proteins (MBNL2 and MBNL3) recapitulate most of the major clinical manifestations of DM in muscle and heart, providing a more representative mouse model of DM in those tissues. In mammalian development MBNL proteins are repressed in embryonic stem cells (ESCs), but increased in cells in culture, such as in HEK293T cells, and in a wide diversity of adult tissues including brain, muscle, liver, etc., where they act to repress a program of splicing found in ESCs. MBNL1 expression can compensate for satellite cell proliferation defects in both primary satellite stem cells and myogenic precursors made from DM1 iPSCs. MBNL proteins regulate splicing of a highly diverse set of gene transcripts, including genes whose protein function as gene expression regulators in differentiation and to control of cytoskeletal dynamics, act as transcription factors, kinases, cellular receptors, and ion channels. MBNL and CELF proteins act antagonistically to specify different cellular outcomes for a set of pre-mRNAs and compete with one another to determine the localization and stability of specific mRNAs that contain binding motifs for both factors. In cardiac tissue MBNL1 acts to antagonize the differentiation program in developing mouse heart induced by CELF1 proteins. Thus, MBNL proteins contribute to an organismal developmental and cellular program through their activity as splicing regulatory factors.

### MBNL1 as a Therapy for DM

Early evaluation of MBNL1 overexpression was attempted in the HSA^{LR} mouse model of DM1. The HSA^{LR} mouse was established as the first definitive functional proof in vivo that repeat expansion was the primary cause of DM. The CTG expansion was engineered in the human alpha skeletal actin gene (HSA or ACTA1) in similar 3′UTR location as in the DMPK gene in human disease. This transgenic mouse line recapitulated some of the characteristics of disease including myotonia, splicing alterations, nuclear foci with MBNL1 and repeat expanded HSA mRNA, and histological changes. Since the HSA gene was expressed in skeletal muscle, none of the cardiac, neurological, or other systemic features of associated with DM1 were present. Muscle histology showed central nucleation and loss of muscle fibers, but the histological phenotype originally seen was lost over generations of breeding.

In the earliest attempt to ameliorate the DM phenotype MBNL1 overexpression was attempted using the HSA^{LR} mice before histological changes were lost in the HSA^{LR} skeletal muscle. Local gene delivery in the tibialis anterior (TA) muscle was successful using adeno-associated viral vector serotype 1 (AAV1) at a dose of 1×10e¹¹ vector genomes (vgs) in 4-5-week-old HSA^{LR} mice. The MBNL1 gene was expressed from chicken β-actin promoter driving expression of the MBNL1 mRNA to produce a myc-tagged MBNL1 41 kd protein. Despite a 20% reduction of the endogenous MBNL1 40 kd protein, there was an overall 2-fold increase in total MBNL1 protein after 23 weeks compared to uninjected mice. Expression of MBNL1 was accompanied by an approximately 60% reduction in myotonia and reversal of splicing defects caused by MBNL1 activity reduction due to repeat expanded HSA mRNA sequestration of MBNL1 in nuclear foci in the TA myofibers of the HSA^{LR} mice. Immunofluorescence (IF) detection of MBNL1 protein showed a redistribution from punctate staining to a more diffuse cloud-like pattern in the injected mice. IF detection of CLCN1 protein, a chloride channel reduced in the HSA^{LR} mice and in DM muscle, showed restoration of the protein to the myofiber membrane. Of note was the lack of correction of the skeletal muscle histological phenotype in the HSA^{LR} mice, when histological changes were still present in the line.

CELF1 expression levels have also been linked to changes in muscle tissue. Data acquired from characterization of a mouse model overexpressing CELF1, which increases with decreasing nuclear MBNL1 protein due to sequestration in foci, demonstrated defects in muscle cell structure and function visualized by changes in muscle histology. Transgenic mice with 8-fold induction of CELF1 expression in adult mice exhibited an overlapping phenotype with DM1 muscle, including dystrophic muscle histology, decreased muscle weight, and splicing alterations in a subset of mRNAs also misregulated in human DM1 skeletal muscle.

In a second model of CELF1 overexpression, muscle histological changes were also seen, as well as fiber type switching and delayed muscle development associated with increases in proteins that are targets of CELF1 translational control, p21 and MEF2A. In contrast, lack of CELF1 in CELF1 knockout mice led to an improvement in dystrophic muscle histology and function with inducible expression of toxic CTG repeats but did not correct spicing defects. Also, overexpression of CELF1 reproduced the muscle damage observed in DM. Thus, it is evident that reduction of CELF1 in the context of toxic repeat mRNA expression in DM may not be able to reverse splicing misregulation but may be beneficial for correction of muscle integrity and functional defects of the disease. These data further support a therapy that would reduce the toxic RNA and/or increase MBNL1 to lead to a reduction in CELF1 for better muscle function.

Two different lines of evidence support the safety of MBNL1 expression, including transgenic expression of MBNL1 (without controlled induction; expression during development and throughout lifespan) and a transgenic cross between MBNL1 overexpressing mice (OE) and HSA^{LR} mice (Chamberlain C M, Ranum L P. Mouse model of muscleblind-like 1 overexpression: skeletal muscle effects and therapeutic promise. Hum Mol Genet. 2012 Nov. 1; 21 (21): 4645-54. doi: 10.1093/hmg/dds306. Epub 2012 Jul. 30. PMID: 22846424; PMCID: PMC3471398). The only indication that ˜8-fold over expression was detrimental in any way was in an increase in mortality of ˜25% at 76 weeks (1.4 yrs) of age, although at no point was a histological or functional examination of the heart performed to assess potential cardiac tissue damage. A cross of the two MBNL1 lines (1 of which minimally overexpressed MBNL1 in the heart with CMV promoter/striated muscle enhancer expression) to the HSA^{LR} mouse resulted in improvements in misregulated splicing and muscle integrity and function. These data suggested that MBNL1 overexpression could be a possible approach for developing a MBNL1-based therapy for DM, although there was still concern stemming from the AAV CMV MBNL1 local muscle expression studies that suggested the muscular dystrophy phenotype was unchanged or worsened by the 2× overexpression of MBNL1 in the HSA^{LR} mice.

More concerning data emerged later from a cross between a low-level, expanded repeat DMPK mRNA (3′ end of gene) expressing mouse, the RNA repeat inducible DM200 mouse. DM200 is an inducible/reversible mouse model of RNA toxicity in which over-expression of an eGFP-DMPK3′UTR (CUG) 200 mRNA results in many DM1 features including myotonia, RNA foci, RNA splicing defects and progressive cardiac conduction defects. A cross between the MBNL1 overexpressing mouse with heart overexpression DM200 mouse line (some DMPK repeat mRNA expressed without induction) led to cardiomegaly and disfunction resulting in early death. A cross of the DM200 mouse with the MBNL1 overexpressing mouse with low expression in the heart was viable, but upon induction of higher levels of expanded repeat DMPK mRNA minimal correction of the splicing phenotype and no reduction in myotonic discharges was observed. Of concern was the decline in muscle structural features with evidence of increased regeneration usually attributable to muscle damage and repair processes.

Some studies have reported that hyperactivation of the autophagy pathway in Drosophila and human DM1 cell models of disease and that the inhibition of this pathway could potentially restore muscle mass and function. More recent evidence describes the use of chloroquine to upregulate Muscleblind mRNA and protein in Drosophila and MBNL in a human DM1 cell model, demonstrating that inhibition of autophagy by chloroquine is a mechanism that releases MBNL1 from autophagosomes to allow build up in cells rather than degradation by fusion with lysosomes. Chloroquine testing in the HSA^{LR} mice required higher doses and this resistance to treatment was attributed to a lack of autophagy hyperactivation in this mouse model.

Taking this a step further, a mouse model expressing expanded repeats in the context of DMPK mRNA versus HSA may have different effects on cellular pathways that may influence the therapeutic response to MBNL1 overexpression in the context of DM1 disease pathology, similar to the differences in autophagy hyperactivation status in the HSA^{LR} mouse compared to Drosophila and human DM1 cell models. Considering the differences in response to MBNL1 overexpression in both the transgenic MBNL1 overexpressing mouse and the HSA^{LR} mouse with phenotypic improvements contrasted with the DM200 mouse, with little improvement in disease features and the potential for muscle toxicity, MBNL1 expression regulation seems to be a critical target for use as a therapy for DM. A critical consideration for therapy that involves treating the heart is the status of the tissue in disease. The heart is affected in DM1, with cardiac arrhythmias and heart block occurring, such that every effort should be made to improve cardiac function.

In accordance with the foregoing, in one aspect the disclosure provides therapeutic compositions.

### First Nucleic Acid

The compositions disclosed herein include at least one nucleic acid construct comprising a first nucleic acid sequence encoding a therapeutic protein. In some embodiments, the first nucleic acid sequence encodes for a MBNL protein (SEQ ID NO:1). Exemplary nucleic acids encoding MBNL protein are set forth in GenBank Accession No. NM_001376830 (SEQ ID NO: 9) and NM_001382683.1 (SEQ ID NO: 10) (Table 4). The first nucleic acid sequence encoding MBNL protein may have at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% sequence identity) to the nucleic acid sequence of SEQ ID NO: 9. The first nucleic acid sequence encoding MBNL protein may have at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% sequence identity) to the nucleic acid sequence of SEQ ID NO: 10.

The first nucleic acid sequence may encode a non-naturally occurring MBNL protein. The non-naturally occurring protein may be derived from the MBNL1 gene, optionally the non-naturally occurring MBNL1 protein lacks a functional domain encoded by exon 1 comprising the major part of the 5′UTR and downstream pre-mRNA introns, that could bind MBNL1 protein for autoregulation, of a wild-type muscleblind-like protein 1 mRNA. Alternatively, the non-naturally occurring protein may be derived from MBNL1 and lacks a functional domain encoded by exon 1 comprising the major part of the 5′UTR and downstream MBNL1 pre-mRNA introns, that can bind MBNL1 protein for autoregulation, of a wild-type Muscleblind-like protein 1 gene, and wherein the non-naturally occurring MBNL1 protein optionally further lacks a functional domain encoded by intron 2 of the wild-type Muscleblind-like protein 1 gene.

The therapeutic composition may comprise an expression vector comprising the at least one nucleic acid construct comprising a first nucleic acid sequence encoding a MBNL protein, such as a viral vector. For example, described herein are adeno-associated viral (AAV) vectors, such as pseudotyped AAV vectors (e.g., AAV2/8 and AAV2/9 vectors) containing transgenes encoding the MBNL proteins described herein that can express MBNL protein. Thus, the compositions disclosed herein include expression vectors comprising the at least one nucleic acid construct comprising a first nucleic acid sequence encoding a MBNL protein. The compositions disclosed herein include expression vectors comprising the at least one nucleic acid construct comprising a first nucleic acid sequence encoding a MBNL1 protein. In some embodiments, the first nucleic acid sequence encodes a non-naturally occurring MBNL protein, as described herein, and the composition comprises an expression vector comprising the first nucleic acid sequence encoding the non-naturally occurring MBNL. In some embodiments, the expression vector comprises a muscle specific promoter. In some embodiments, the promoter comprises CK8e and the like or a ubiquitous promoter. In some embodiments, the expression vector is a recombinant adenoviral vector.

The compositions and methods described herein may selectively increase the presence of a functional MBNL1 protein expression. As used herein, the term “functional MBNL” or “functional MBNL1” refers to MBNL protein that is not bound to a CTG microsatellite repeat in the 3′ UTR of a nucleic acid encoding DMPK. The increase in functional MBNL1 protein expression may be an increase of, for example, about 1% or more, such as an increase of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%, relative to the expression of MBNL1 in a subject prior to administration of a therapeutic agent described herein, such as a vector or nucleic acid described herein. Methods that can be used to assess protein expression levels are known in the art and include western blotting, immunoprecipitation, and other techniques described herein.

### Second Nucleic Acid

The therapeutic compositions described herein may further include a nucleic acid construct comprising a second nucleic acid sequence encoding an interfering RNA constructs that suppresses the expression of RNA transcripts containing aberrantly expanded repeat regions, such as siRNA, miRNA, and shRNA constructs that anneal to portions of nuclear-retained, repeat-expanded RNA transcripts and promote the degradation of these pathological transcripts by way of various cellular processes. The interfering RNAs described herein may be in any of a variety of forms, such as short interfering RNA (siRNA), short hairpin RNA (shRNA), or micro-RNA (miRNA). The interfering RNAs described herein may additionally be encoded by a vector, such as a viral vector. For example, described herein are adeno-associated viral (AAV) vectors, such as pseudotyped AAV vectors (e.g., AAV2/8 and AAV2/9 vectors) containing transgenes encoding interfering RNA constructs that attenuate the expression of RNA transcripts harboring expanded nucleotide repeats.

Exemplary interfering RNAs encoded by the second nucleic acid sequence are disclosed in U.S. Patent Publication No. US20210269825A1, incorporated herein by reference in its entirety.

Myotonic dystrophy patients that may be treated using the compositions and methods described herein include patients, such as human patients, having myotonic dystrophy type I, and that express a DMPK RNA transcript harboring a CUG repeat expansion. Exemplary DMPK RNA transcripts that may be expressed by a patient undergoing treatment with the compositions and methods described herein are set forth in GenBank Accession Nos. NM_001081560.1, NT_011109.15 (from nucleotides 18540696 to Ser. No. 18/555,106), NT_039413.7 (from nucleotides 16666001 to Ser. No. 16/681,000), NM_032418.1, AI007148.1, AI304033.1, BC024150.1, BC056615.1, BC075715.1, BU519245.1, CB247909.1, CX208906.1, CX732022.1, 560315.1, 560316.1, NM_001081562.1, and NM_001100.3.

The portion of each silencing/interfering RNA(s) encoded by the second nucleic acid sequence may anneal to a segment of the endogenous mRNA transcript that does not overlap with the expanded repeat region.

In some embodiments, the endogenous mRNA transcript encodes human DMPK and contains an expanded repeat region. The expanded repeat region may contain, for example, 50 or more CUG trinucleotide repeats, such as from about 50 to about 4,000 CUG trinucleotide repeats (e.g., about 50 CUG trinucleotide repeats, about 60 CUG trinucleotide repeats, about 70 trinucleotide repeats, 80 trinucleotide repeats, 90 trinucleotide repeats, 100 trinucleotide repeats, 110 trinucleotide repeats, 120 trinucleotide repeats, 130 trinucleotide repeats, 140 trinucleotide repeats, 150 trinucleotide repeats, 160 trinucleotide repeats, 170 trinucleotide repeats, 180 trinucleotide repeats, 190 trinucleotide repeats, 200 trinucleotide repeats, 210 trinucleotide repeats, 220 trinucleotide repeats, 230 trinucleotide repeats, 240 trinucleotide repeats, 250 trinucleotide repeats, 260 trinucleotide repeats, 270 trinucleotide repeats, 280 trinucleotide repeats, 290 trinucleotide repeats, 300 trinucleotide repeats, 310 trinucleotide repeats, 320 trinucleotide repeats, 330 trinucleotide repeats, 340 trinucleotide repeats, 350 trinucleotide repeats, 360 trinucleotide repeats, 370 trinucleotide repeats, 380 trinucleotide repeats, 390 trinucleotide repeats, 400 trinucleotide repeats, 410 trinucleotide repeats, 420 trinucleotide repeats, 430 trinucleotide repeats, 440 trinucleotide repeats, 450 trinucleotide repeats, 460 trinucleotide repeats, 470 trinucleotide repeats, 480 trinucleotide repeats, 490 trinucleotide repeats, 500 trinucleotide repeats, 510 trinucleotide repeats, 520 trinucleotide repeats, 530 trinucleotide repeats, 540 trinucleotide repeats, 550 trinucleotide repeats, 560 trinucleotide repeats, 570 trinucleotide repeats, 580 trinucleotide repeats, 590 trinucleotide repeats, 600 trinucleotide repeats, 610 trinucleotide repeats, 620 trinucleotide repeats, 630 trinucleotide repeats, 640 trinucleotide repeats, 650 trinucleotide repeats, 660 trinucleotide repeats, 670 trinucleotide repeats, 680 trinucleotide repeats, 690 trinucleotide repeats, 700 trinucleotide repeats, 710 trinucleotide repeats, 720 trinucleotide repeats, 730 trinucleotide repeats, 740 trinucleotide repeats, 750 trinucleotide repeats, 760 trinucleotide repeats, 770 trinucleotide repeats, 780 trinucleotide repeats, 790 trinucleotide repeats, 800 trinucleotide repeats, 810 trinucleotide repeats, 820 trinucleotide repeats, 830 trinucleotide repeats, 840 trinucleotide repeats, 850 trinucleotide repeats, 860 trinucleotide repeats, 870 trinucleotide repeats, 880 trinucleotide repeats, 890 trinucleotide repeats, 900 trinucleotide repeats, 910 trinucleotide repeats, 920 trinucleotide repeats, 930 trinucleotide repeats, 940 trinucleotide repeats, 950 trinucleotide repeats, 960 trinucleotide repeats, 970 trinucleotide repeats, 980 trinucleotide repeats, 990 trinucleotide repeats, 1,000 trinucleotide repeats, 1,100 trinucleotide repeats, 1,200 trinucleotide repeats, 1,300 trinucleotide repeats, 1,400 trinucleotide repeats, 1,500 trinucleotide repeats, 1,600 trinucleotide repeats, 1,700 trinucleotide repeats, 1,800 trinucleotide repeats, 1,900 trinucleotide repeats, 2,000 trinucleotide repeats, 2,100 trinucleotide repeats, 2,200 trinucleotide repeats, 2,300 trinucleotide repeats, 2,400 trinucleotide repeats, 2,500 trinucleotide repeats, 2,600 trinucleotide repeats, 2,700 trinucleotide repeats, 2,800 trinucleotide repeats, 2,900 trinucleotide repeats, 3,000 trinucleotide repeats, 3,100 trinucleotide repeats, 3,200 trinucleotide repeats, 3,300 trinucleotide repeats, 3,400 trinucleotide repeats, 3,500 trinucleotide repeats, 3,600 trinucleotide repeats, 3,700 trinucleotide repeats, 3,800 trinucleotide repeats, 3,900 trinucleotide repeats, or 4,000 trinucleotide repeats, among others).

In some embodiments, the endogenous mRNA transcript contains a portion having at least 85% sequence identity (e.g., 85%, 85%, 87%, 88%, 89%, 90%, 91%, 92%, 93%, 94%, 95%, 96%, 97%, 98%, 99%, 99.9%, or 100% sequence identity) to the nucleic acid sequence of SEQ ID NO: 7 or SEQ ID NO: 8 (Table 3). In some embodiments, the endogenous RNA transcript contains a portion having at least 90% sequence identity (e.g., 90%, 91%, 92%, 93%, 94%, 95%, 96%, 97%, 98%, 99%, 99.9%, or 100% sequence identity) to the nucleic acid sequence of SEQ ID NO: 7 or SEQ ID NO: 8. In some embodiments, the endogenous mRNA transcript contains a portion having at least 95% sequence identity (e.g., 95%, 96%, 97%, 98%, 99%, 99.9%, or 100% sequence identity) to the nucleic acid sequence of SEQ ID NO: 7 or SEQ ID NO: 8. The endogenous mRNA transcript may contain, for example, a portion having the nucleic acid sequence of SEQ ID NO: 7 or SEQ ID NO: 8.

The interfering RNA(s) encoded by the second nucleic acid sequence may be at least 5, at least 10, at least 17, at least 19, or more, nucleotides in length, (e.g., at least 5, 6, 7, 8, 9, 10, 1 1, 12, 13, 14, 15, 16, 17, 18, 19, or more, nucleotides in length, such as from 17 to 24, 18 to 23, or 19 to 22 nucleotides in length). The interfering RNA(s) may, e.g., each be, independently, from 10-35 nucleotides in length. In some embodiments, the interfering RNA(s) are 10 nucleotides in length. In some embodiments, the interfering RNA(s) are 11 nucleotides in length. In some embodiments, the interfering RNA(s) are 12 nucleotides in length. In some embodiments, the interfering RNA(s) are 13 nucleotides in length. In some embodiments, the interfering RNA(s) are 14 nucleotides in length. In some embodiments, the interfering RNA(s) are 15 nucleotides in length. In some embodiments, the interfering RNA(s) are 16 nucleotides in length. In some embodiments, the interfering RNA(s) are 17 nucleotides in length. In some embodiments, the interfering RNA(s) are 18 nucleotides in length. In some embodiments, the interfering RNA(s) are 19 nucleotides in length. In some embodiments, the interfering RNA(s) are 20 nucleotides in length. In some embodiments, the interfering RNA(s) are 21 nucleotides in length. In some embodiments, the interfering RNA(s) are 22 nucleotides in length. In some embodiments, the interfering RNA(s) are 23 nucleotides in length. In some embodiments, the interfering RNA(s) are 24 nucleotides in length. In some embodiments, the interfering RNA(s) are 25 nucleotides in length. In some embodiments, the interfering RNA(s) are 26 nucleotides in length. In some embodiments, the interfering RNA(s) are 27 nucleotides in length. In some embodiments, the interfering RNA(s) are 28 nucleotides in length. In some embodiments, the interfering RNA(s) are 29 nucleotides in length. In some embodiments, the interfering RNA(s) are 30 nucleotides in length. In some embodiments, the interfering RNA(s) are 31 nucleotides in length. In some embodiments, the interfering RNA(s) are 32 nucleotides in length. In some embodiments, the interfering RNA(s) are 33 nucleotides in length. In some embodiments, the interfering RNA(s) are 34 nucleotides in length. In some embodiments, the interfering RNA(s) are 35 nucleotides in length.

In some embodiments, the interfering RNA(s) contain a portion that anneals to an endogenous RNA transcript containing an expanded repeat region. The portion of each interfering RNA(s) may anneal to a segment of the endogenous RNA transcript that does not overlap with the expanded repeat region.

The compositions and methods described herein may selectively attenuate the expression of DMPK mRNA transcripts containing expanded CUG repeats, such as DMPK mRNA transcripts containing from about 50 to about 4,000, or more, CUG repeats. For example, the interfering RNA molecules described herein may activate ribonucleases, such as nuclear ribonucleases, that specifically digest nuclear-retained DMPK transcripts harboring CUG repeat expansions. The decrease in mutant DMPK mRNA expression may be a decrease of, for example, about 1% or more, such as a decrease of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%, relative to the expression of DMPK mRNA transcripts containing expanded CUG trinucleotide repeat regions by the patient prior to administration of a therapeutic agent described herein, such as a vector or nucleic acid described herein. Methods that can be used to assess RNA expression levels are known in the art and include RNA-seq assays and polymerase chain reaction techniques described herein.

In some embodiments, the therapeutic composition comprises at least one nucleic acid construct comprising the first nucleic acid sequence and the second nucleic acid sequence. In some embodiments, the therapeutic composition comprises expression vectors comprising the at least one nucleic acid construct comprising the first nucleic acid sequence and the second nucleic acid sequence.

In some embodiments, the first nucleic acid sequence and the second nucleic acid sequence are in the same expression cassette or expression vector and are operatively linked to the same first promoter. In some embodiments, the first nucleic acid sequence and the second nucleic acid sequence are in different expression cassettes or expression vectors, wherein the first nucleic acid sequence is operatively linked to a first promoter and second nucleic acid sequence is operatively linked to a second promoter. In some embodiments, the first promoter is active in a skeletal muscle cell. In some embodiments, the first promoter is or comprises CK8e and the like. In some embodiments, the second promoter is or comprises an RNA Pol III promoter (e.g., U6 promoter) sequence or RNA Pol II promoter (e.g., CK8) sequence.

### Third Nucleic Acid

The therapeutic compositions disclosed herein comprising at least one nucleic acid construct may further include a third nucleic acid sequence encoding a regulatory element useful for controlling/regulating and/or directing tissue-specific expression of the at least one nucleic acid construct described herein. In some embodiments, the third nucleic acid sequence is operatively linked to the first nucleic acid sequence. In some embodiments, the regulatory element is useful in controlling and/or directing tissue-specific expression of the therapeutic protein. In some embodiments, the third nucleic acid sequence encodes a binding or target site for a cardiac miRNA. In some embodiments, the cardiac miRNA is a miRNA expressed in cardiac muscle cells. In some embodiments, the cardiac miRNA is a miRNA expressed exclusively or predominantly in cardiac muscle cells. In some embodiments, the cardiac miRNA is miR208a. In some embodiments, association of the cardiac miRNA to the third nucleic acid sequence prevents or reduces expression of MBNL from the first nucleic acid.

In some embodiments, the therapeutic composition comprises at least one nucleic acid construct comprising the first nucleic acid sequence, the second nucleic acid sequence, and the third nucleic acid sequence. In some embodiments, the therapeutic composition comprises an expression vector comprising at least one nucleic acid construct comprising the first nucleic acid sequence, the second nucleic acid sequence, and the third nucleic acid sequence.

In some embodiments, the therapeutic composition comprises at least one nucleic acid construct comprising the first nucleic acid sequence and the third nucleic acid sequence. In some embodiments, the therapeutic composition comprises a first expression vector comprising at least one nucleic acid construct comprising the first nucleic acid sequence and the third nucleic acid sequence operatively linked to a first promoter. In some embodiments, the therapeutic composition further comprises a second expression vector comprising at least one nucleic acid construct comprising the second nucleic acid sequence operatively linked to a second promoter. In some embodiments, the first promoter is active in a skeletal muscle cell. In some embodiments, the first promoter is or comprises CK8e and the like. In some embodiments, the second promoter is or comprises an RNA Pol III promoter (e.g., U6 promoter) sequence or RNA Pol II promoter (e.g., CK8) sequence.

The compositions and methods described herein may selectively drive tissue specific expression of the at least one nucleic acid construct described herein.

### Fourth Nucleic Acid

In some embodiments, the therapeutic composition comprising the at least one nucleic acid construct further comprises a fourth nucleic acid sequence. In some embodiment, the fourth nucleic acid sequence comprises a chimeric intron with beta-globin (b-globin or β-globin) and immunoglobulin domains. In some embodiments, the chimeric intron serves as an MBLN1 binding site. In some embodiments, the chimeric intron serves to autoregulate MBNL1 expression. In some embodiments, the chimeric intron serves to enhance MBLN1 expression. In some embodiments, the fourth nucleic acid sequence is operatively linked to the first nucleic acid sequence.

In some embodiments, the therapeutic composition comprises at least one nucleic acid construct comprising the first nucleic acid sequence, the third nucleic acid sequence, and the fourth nucleic acid sequence. In some embodiments, the third nucleic acid sequence is operatively linked to the first nucleic acid sequence. In some embodiments, the fourth nucleic acid sequence and the third nucleic acid sequence are operatively linked to the first nucleic acid sequence.

In some embodiments, the therapeutic composition comprises a first expression vector comprising the first nucleic acid sequence, the third nucleic acid sequence, and a fourth nucleic acid sequence. In some embodiments, the third and the first nucleic acid sequences are operatively connected to a first promoter. In some embodiments, the therapeutic composition further comprises a second expression vector comprising the second nucleic acid sequence operatively linked to a second promoter. In some embodiments, the first promoter is active in a skeletal muscle cell. In some embodiments, the first promoter is or comprises CK8e and the like. In some embodiments, the second promoter is or comprises an RNA Pol III promoter (e.g., U6 promoter) sequence or RNA Pol II promoter (e.g., CK8) sequence.

In some embodiments, the composition comprises at least one nucleic acid construct comprising the first nucleic acid sequence, the second nucleic acid sequence, and the third nucleic acid sequence. In some embodiments, the composition comprises at least one nucleic acid construct comprising the first nucleic acid sequence and the third nucleic acid sequence.

In some embodiments, the composition comprises a vector comprising the at least one nucleic acid construct comprising the first nucleic acid sequence and the third nucleic acid sequence.

In some embodiments, the nucleic acid construct further comprises a fourth nucleic acid sequence operatively linked to the first nucleic acid sequence, wherein the fourth nucleic acid sequence comprises a chimeric intron with beta-globin (b-globin or β-globin) and immunoglobulin domains. In some embodiments, the chimeric intron serves as an MBLN1 binding site. In some embodiments, the chimeric intron serves to autoregulate MBNL1 expression. In some embodiments, the chimeric intron serves to enhance MBLN1 expression.

The compositions disclosed herein include expression vector(s) comprising the nucleic acid constructs comprising the nucleic acid sequences disclosed herein. The expression vectors disclosed herein comprise at least one promoter operably linked to at least one nucleic acid sequences of the present disclosure, and capable of driving the transcription of the at least one nucleic acid sequence of the present disclosure. In some embodiments, the first nucleic acid sequence (and optionally third and/or fourth nucleic acid sequences) and the second nucleic acid sequence are present on separate expression vector constructs. In some embodiments, the first nucleic acid sequence (and optionally third and/or fourth nucleic acid sequences) and the second nucleic acid sequence are present on the same expression vector construct. In some embodiments, the first nucleic acid sequence (and optionally third and/or fourth nucleic acid sequences) and the second nucleic acid sequence are operatively linked to the same promoter (e.g., CK8e and the like). In some embodiments, the first nucleic acid sequence (and optionally third and/or fourth nucleic acid sequences) and the second nucleic acid sequence are operatively linked to separate promoter sequences. In some embodiments, the first nucleic acid sequence (and optionally third and/or fourth nucleic acid sequences) are operatively linked to a CK8e promoter sequence and the second nucleic acid sequence is operatively linked to an RNA Pol III promoter (e.g., U6 promoter) sequence or RNA Pol II promoter (e.g., CK8) sequence.

In some embodiments, the at least one nucleic acid construct is present in a viral vector, e.g., AAV vector.

Another aspect of the disclosure provides a method of producing a viral vector. e.g., recombinant AAV vector (rAAV) of the present disclosure, comprising culturing a cell that has been transfected with any viral vector. e.g., rAAV vector of the disclosure and recovering the virus, e.g., rAAV particles from the supernatant of the transfected cells. Another aspect of the invention provides viral particles comprising any of the viral vectors. e.g., recombinant AAV vectors of the present disclosure.

Another aspect of the present disclosure provides a composition comprising any of the expression vectors of the present disclosure, e.g., the recombinant viral (AAV) vector of the present disclosure.

The present disclosure also contemplates use of any of the expression vector, e.g., AAV vectors of the disclosure for the preparation of a medicament for administering any of the expression vectors, e.g., rAAV of the disclosure to a subject suffering from muscular dystrophy.

In certain embodiments, the composition is a pharmaceutical composition further comprising a therapeutically compatible carrier, diluent, or excipient. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG). In certain embodiments, the therapeutically acceptable carrier, diluent, or excipient is a sterile aqueous solution comprising 10 mM L-histidine at pH 6.0, 150 mM sodium chloride, and 1 mM magnesium chloride.

In certain embodiments, the vector is an AAV1, AAV2, AAV4, AAV5, AAV6, AAV7, AAVrh74, AAV8, AAV9, AAV 10, AAV 11, AAV 12, or AAV 13 vector. In a related aspect the present disclosure provides a method of increasing the presence of functional muscleblind-like protein (MBNL) in the nucleus of a cell. The method comprises contacting the cell with at least one nucleic acid construct comprising the first nucleic acid sequence, described herein. In some embodiments, the first nucleic acid sequence encodes a functional MBNL protein. In some embodiments, the MBNL is characterized as functional when it is not bound to a CTG microsatellite repeat in the 3′ UTR of a nucleic acid encoding DMPK. In some embodiments, the MBNL is MBNL1. In some embodiments, the cell is a muscle cell. In some embodiments, the muscle cell is a skeletal muscle cell.

In some embodiments, the first nucleic acid sequence encodes a non-naturally occurring MBNL protein. In some embodiments, the non-naturally occurring protein is derived from the MBNL1 gene. In some embodiments, the non-naturally occurring protein lacks a functional domain encoded by exon 1 comprising the major part of the 5′UTR and downstream pre-mRNA introns, that could bind MBNL1 protein for autoregulation, of a wild-type Muscleblind-like protein 1 mRNA. In some embodiments, the non-naturally occurring protein is derived from MBNL1. In some embodiments, the non-naturally occurring protein derived from MBNL1 lacks a functional domain encoded by exon 1 comprising the major part of the 5′UTR and downstream MBNL1 pre-mRNA introns, that can bind MBNL1 protein for autoregulation, of a wild-type Muscleblind-like protein 1 gene. In some embodiments, the non-naturally occurring protein further lacks a functional domain encoded by intron 2 of the wild-type Muscleblind-like protein 1 gene.

In some embodiments of the methods disclosed herein, the at least one nucleic acid construct comprises a second nucleic acid sequence encoding a silencing/interfering RNA, as described herein. In some embodiments, the silencing/interfering RNA(s) contain a portion that anneals to an endogenous RNA transcript containing an expanded repeat region. In some embodiments, the silencing/interfering RNA hybridizes to an mRNA transcript encoding dystrophia myotonica protein kinase (DMPK). Thus, in some embodiments, the methods disclosed herein comprise contacting the cell with at least one nucleic acid construct comprising a first nucleic acid sequence encoding a functional MBNL protein and a second nucleic acid sequence encoding a silencing RNA that hybridizes to an mRNA encoding dystrophia myotonica protein kinase (DMPK).

In some embodiments, the silencing RNA is a microRNA (miRNA), or any small RNA generating and RNAi pathway engaging and activating RNA that, upon hybridizing to the mRNA encoding dystrophia myotonica protein kinase (DMPK) reduces the level of the DMPK mRNA and reduces translation of DMPK protein and cytotoxic proteins, such as repeat-associated non-AUG (RAN) translation products, from the expanded repeat-containing mRNA.

In some embodiments of the methods disclosed herein, the first nucleic acid sequence is operatively linked to a third nucleic acid sequence, as described herein. In some embodiments, the third nucleic acid encodes a binding site for a cardiac miRNA.

In some embodiments, the methods disclosed herein comprise contacting the cell with at least one nucleic acid construct comprising a first nucleic acid sequence encoding a functional MBNL protein and a third nucleic acid encoding a binding site for a cardiac miRNA.

In some embodiments, the methods disclosed herein comprise contacting the cell with at least one nucleic acid construct comprising a first nucleic acid sequence encoding a functional MBNL protein, a second nucleic acid sequence encoding a silencing RNA that hybridizes to an mRNA encoding dystrophia myotonica protein kinase (DMPK), and a third nucleic acid encoding a binding site for a cardiac miRNA.

In some embodiments, the cardiac miRNA is a miRNA expressed in cardiac muscle cells. In some embodiments, the cardiac miRNA is a miRNA expressed exclusively or predominantly in cardiac muscle cells. In some embodiments, the cardiac miRNA is miR208a.

In some embodiments of the methods disclosed herein, the first nucleic acid sequence is operatively linked to a fourth nucleic acid sequence. In some embodiments, the fourth nucleic acid sequence comprises a chimeric intron with beta-globin (b-globin or β-globin) and immunoglobulin sequences. In some embodiments, the chimeric intron serves as an MBLN1 binding site. In some embodiments, the chimeric intron serves to autoregulate MBNL1 expression. In some embodiments, the chimeric intron serves to enhance MBLN1 expression.

In some embodiments, the methods disclosed herein comprise contacting the cell with at least one nucleic acid construct comprising a first nucleic acid sequence encoding a functional MBNL protein, a third nucleic acid encoding a binding site for a cardiac miRNA, and a fourth nucleic acid sequence comprising a chimeric intron with beta-globin (b-globin or β-globin) and immunoglobulin sequences. In some embodiments, the chimeric intron serves as an MBLN1 binding site. In some embodiments, the chimeric intron serves to autoregulate MBNL1 expression. In some embodiments, the chimeric intron serves to enhance MBLN1 expression.

In some embodiments, the methods disclosed herein comprise contacting the cell with at least one nucleic acid construct comprising a first nucleic acid sequence encoding a functional MBNL protein, a second nucleic acid sequence encoding a silencing RNA that hybridizes to an mRNA encoding dystrophia myotonica protein kinase (DMPK), a third nucleic acid encoding a binding site for a cardiac miRNA, and a fourth nucleic acid sequence comprising a chimeric intron with beta-globin (b-globin or β-globin) and immunoglobulin sequences. In some embodiments, the chimeric intron serves as an MBLN1 binding site. In some embodiments, the chimeric intron serves to autoregulate MBNL1 expression. In some embodiments, the chimeric intron serves to enhance MBLN1 expression.

In some embodiments, the first nucleic acid sequence (and optionally third and/or fourth nucleic acid sequences) and the second nucleic acid sequence are present on separate expression vector constructs. In some embodiments, the first nucleic acid sequence (and optionally third and/or fourth nucleic acid sequences) and the second nucleic acid sequence are present on the same expression vector construct. In some embodiments, the first nucleic acid sequence (and optionally third and/or fourth nucleic acid sequences) and the second nucleic acid sequence are operatively linked to the same promoter (e.g., CK8e and the like). In some embodiments, the first nucleic acid sequence (and optionally third and/or fourth nucleic acid sequences) and the second nucleic acid sequence are operatively linked to separate promoter sequences. In some embodiments, the first nucleic acid sequence (and optionally third and/or fourth nucleic acid sequences) are operatively linked to a CK8e promoter sequence and the second nucleic acid sequence is operatively linked to an RNA Pol III promoter (e.g., U6 promoter) sequence or RNA Pol II promoter (e.g., CK8) sequence.

In some embodiments, the at least one nucleic acid construct is present in a viral vector, e.g., AAV vector.

In some embodiments, the cell is in vitro. In some embodiments, the cell in vivo in a subject with myotonic dystrophy type 1 (DM1), and wherein the method is a method of treating, ameliorating, or preventing symptoms of DM1.

In some embodiments, the subject is a human, rodent (e.g., mouse or rat), dog, cat, and the like. In some embodiments, expression of the MBNL protein from the first nucleic acid sequence and the silencing RNA from the second nucleic acid sequence results in an increase in functional MBNL protein in nucleic of skeletal muscle cells in the subject. In some embodiments, the first nucleic acid sequence is operatively linked to a third nucleic acid sequence that is a binding site for a cardiac miRNA, wherein the cardiac miRNA is a miRNA expressed (e.g., predominantly or exclusively) in cardiac muscle cells, optionally wherein the cardiac miRNA is miR208a. In some embodiments, association of the cardiac miRNA to the third nucleic acid sequence prevents or reduces expression of MBNL from the first nucleic acid.

Using the compositions and methods described herein, a subject, such as a subject suffering from myotonic dystrophy (e.g., myotonic dystrophy type I) may be administered one or more vectors encoding the nucleic acid constructs disclosed herein, or may be administered the compositions disclosed herein.

In another aspect, the disclosure provides a nucleic acid construct. The construct comprises a first nucleic acid sequence encoding Muscleblind like protein (MBNL); and a second nucleic acid sequence encoding a silencing RNA that hybridizes to an mRNA encoding dystrophia myotonica protein kinase (DMPK) protein.

In some embodiments, the first nucleic acid sequence and the second nucleic acid sequence are in the same expression cassette and are operatively linked to the same first promoter. In some embodiments, the first nucleic acid sequence and the second nucleic acid sequence are in different expression cassettes, wherein the first nucleic acid sequence is operatively linked to a first promoter and second nucleic acid sequence is operatively linked to a second promoter. In some embodiments, the first promoter is active in a skeletal muscle cell. In some embodiments, the first promoter is or comprises CK8e and the like. In some embodiments, the second promoter is or comprises an RNA Pol III promoter (e.g., U6 promoter) sequence or RNA Pol II promoter (e.g., CK8) sequence.

In some embodiments, the nucleic acid construct further comprises a third nucleic acid sequence that is a binding site for a cardiac miRNA and which is operatively linked to the first nucleic acid sequence. In some embodiments, the cardiac miRNA is a miRNA expressed in cardiac muscle cells. In some embodiments, the cardiac miRNA is a miRNA expressed exclusively or predominantly in cardiac muscle cells. In some embodiments, the cardiac miRNA is miR208a.

In some embodiments, the nucleic acid construct further comprises a fourth nucleic acid sequence operatively linked to the first nucleic acid sequence, wherein the fourth nucleic acid sequence comprises a chimeric intron with beta-globin (b-globin or β-globin) and immunoglobulin domains. In some embodiments, the chimeric intron serves as an MBLN1 binding site.

In another aspect, the disclosure provides a nucleic acid construct comprising: a first nucleic acid sequence encoding muscle blind like protein (MBNL); and a third nucleic acid sequence operatively linked to the first nucleic acid sequence, which is a binding site for a cardiac miRNA.

In some embodiments, the construct further comprises a second nucleic acid sequence encoding a silencing RNA that hybridizes to an mRNA encoding dystrophia myotonica protein kinase (DMPK) protein.

In some embodiments, the first nucleic acid sequence is operatively linked to a first promoter that is active in a skeletal muscle cell. In some embodiments, the first promoter is or comprises CK8e and the like. In some embodiments, the cardiac miRNA is a miRNA expressed in cardiac muscle cells. In some embodiments, the cardiac miRNA is a miRNA expressed in exclusively or predominantly in cardiac muscle cells. In some embodiments, the cardiac miRNA is miR208a. In some embodiments, the nucleic acid construct further comprises a fourth nucleic acid sequence operatively linked to the first nucleic acid sequence, wherein the fourth nucleic acid sequence comprises a chimeric intron with beta-globin (b-globin or β-globin) and immunoglobulin domains. In some embodiments, the chimeric intron serves as an MBLN1 binding site.

The present disclosure is also directed to compositions for treating myotonic dystrophy in a subject in need thereof.

Another aspect of the invention provides a method of treating a muscular dystrophy or spliceopathy in a subject in need thereof, the method comprising administering to the subject a therapeutically effective amount of any one of the expression vectors comprising at least one of the nucleic acid constructs of the disclosure, e.g., the recombinant AAV vector of the disclosure, or any one of the therapeutic compositions of the disclosure. In certain embodiments, there is provided a method of treating myotonic dystrophy type 1 (DM1) in a subject in need thereof, the method comprising administering to the subject a therapeutically effective amount of the recombinant viral vector (e.g., the recombinant AAV vector) of the disclosure, or the pharmaceutical composition comprising such expression vectors or recombinant viral vectors.

For administration, effective amounts, and therapeutically effective amounts (also referred to herein as doses) may be initially estimated based on results from in vitro assays and/or animal model studies. For example, a dose may be formulated in animal models to achieve a circulating concentration range that includes the IC₅₀ as determined in cell culture. Such information may be used to determine useful doses more accurately in subjects of interest. Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the disease state being treated and the target cells/tissue(s) that are to express the expression constructs of the present disclosure.

Specifically, the formulations described herein may be administered by, without limitation, injection, infusion, perfusion, inhalation, lavage, and/or ingestion. Routes of administration may include, but are not limited to, intravenous, intradermal, intraarterial, intraperitoneal, intralesional, intracranial, intraarticular, intraprostatic, intrapleural, intratracheal, intranasal, intravitreal, intravaginal, intrarectal, topically, intratumoral, intramuscular, intravesicular, intrapericardial, intraumbilical, intraocularal, mucosal, oral, subcutaneous, and/or subconjunctival.

In certain embodiments, the expression vector, e.g., the recombinant AAV vector comprising at least one nucleic acid construct, or the pharmaceutical composition of the present disclosure is administered by intramuscular injection, intravenous injection, parental administration, or systemic administration.

Another aspect of the invention provides a kit for preventing or treating a disease, such as DM1 or related/associated diseases, in a subject, the kit comprising: one or more expression vectors, e.g., the recombinant AAV as described herein, or a therapeutic composition as described herein; instructions for use (written, printed, electronic/optical storage media, or online); and/or packaging. In certain embodiments, a kit also includes a known therapeutic composition for treating the disease (e.g., DM1), for combination therapy.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); Coligan, J. E., et al. (eds.), Current Protocols in Immunology, John Wiley & Sons, New York (2010); Mirzaei, H. and Carrasco, M. (eds.), Modern Proteomics-Sample Preparation, Analysis and Practical Applications in Advances in Experimental Medicine and Biology, Springer International Publishing, 2016; Comai, L, et al., (eds.), Proteomic: Methods and Protocols in Methods in Molecular Biology, Springer International Publishing, 2017; Mali P, Esvelt K M, and Church G M. Cas9 as a versatile tool for engineering biology. Nat Methods. 2013 October; 10 (10): 957-63; and Dominguez A A, Lim W A, and Qi L S. Beyond editing: repurposing CRISPR-Cas9 for precision genome regulation and interrogation. Nat Rev Mol Cell Biol. 2016 January; 17 (1): 5-15, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

A nucleic acid is a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyribo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, a ribonucleotide (RNA) polymer. The nucleic acids can also be or comprise a PNA polymer, or a combination of any of the polymer types described herein (e.g., contain residues with different sugars).

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine(S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

Reference to sequence identity addresses the degree of similarity of two polymeric sequences, such as protein or nucleic acid sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

The term “treating” and grammatical variants thereof refer to any indicia of success in the treatment, amelioration, and/or prevention of a disease or condition (e.g., a myotonic dystrophy, e.g., DM1), including any objective or subjective parameter such as abatement, remission, diminishing of symptoms or making the disease condition more tolerable to the patient, slowing in the rate of degeneration or decline, or making the final point of degeneration less debilitating. The treatment or amelioration of symptoms can be based on objective or subjective parameters, including the results of an examination by a physician. Accordingly, the term “treating” includes the administration of compounds or agents to prevent or delay, to alleviate, or to arrest or inhibit development of the symptoms or conditions associated with disease or condition (e.g., a myotonic dystrophy, e.g., DM1). The term “therapeutic effect” refers to the reduction, elimination, slowing, or prevention of the disease or condition, symptoms of the disease or condition, or side effects of the disease or condition in the subject.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “about” refers to a value that is within 10% above or below the value being described. For example, the phrase “about 100 nucleic acid residues” refers to a value of from 90 to 110 nucleic acid residues.

As used herein, the term “anneal” refers to the formation of a stable duplex of nucleic acids by way of hybridization mediated by inter-strand hydrogen bonding, for example, according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another. The “stable duplex” formed upon the annealing of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

As used herein, the terms “dystrophia myotonica protein kinase” and its abbreviation, “DMPK,” refer to the serine/threonine kinase protein involved in the regulation of skeletal muscle structure and function, for example, in human subjects. The terms “dystrophia myotonica protein kinase” and “DMPK” are used interchangeably herein and refer not only to wild-type forms of the DMPK gene, but also to variants of wild-type DMPK proteins and nucleic acids encoding the same. The nucleic acid sequences of two isoforms of human DMPK mRNA are provided herein as SEQ ID NOs: 7 and 8, which correspond to GenBank Accession Nos. BC026328.1 and BC062553.1, respectively (3′ UTRs not included). These nucleic acid sequences are provided in Table 3, below.

TABLE 3
Nucleic acid sequences of exemplary human DMPK isoforms

| SEQ | |
|---|---|
| ID | |
| NO: | Nucleic Acid Sequence |
| 7 | GGCUGGACCAAGGGGUGGGGAGAAGGGGAGGAGGCCUCGGCCGGCCG |
| | CAGAGAGAAGUGGCCAGAGAGGCCCAGGGGACAGCCAGGGACAGGCA |
| | GACAUGCAGCCAGGGCUCCAGGGCCUGGACAGGGGCUGCCAGGCCCU |
| | GUGACAGGAGGACCCCGAGCCCCCGGCCCGGGGAGGGGCCAUGGUGC |
| | UGCCUGUCCAACAUGUCAGCCGAGGUGCGGCUGAGGCGGCUCCAGCA |
| | GCUGGUGUUGGACCCGGGCUUCCUGGGGCUGGAGCCCCUGCUCGACC |
| | UUCUCCUGGGCGUCCACCAGGAGCUGGGCGCCUCCGAACUGGCCCAG |
| | GACAAGUACGUGGCCGACUUCUUGCAGUGGGCCCCAAAUCCAGGGUU |
| | UUCCAAAGUGUGGUUCAAGAACCACCUGCAUCUGAAUCUAGAGCGGA |
| | GCCCAUCGUGGUGAGGCUUAAGGAGGUCCGACUGCAGAGGGACGACU |
| | UCGAGAUUCUGAAGGUGAUCGGACGCGGGGCGUUCAGCGAGGUAGCG |
| | GUAGUGAAGAUGAAGCAGACGGGCCAGGUGUAUGCCAUGAAGAUCA |
| | UGAACAAGUGGGACAUGCUGAAGAGGGGCGAGGUGUCGUGCUUCCGU |
| | GAGGAGAGGGACGUGUUGGUGAAUGGGGACCAGCGGUGGAUCACGC |
| | AGCUGCACUUCGCCUUCCAGGAUGAGAACUACCUGUACCUGGUCAUG |
| | GAGUAUUACGUGGGCGGGGACCUGCUGACACUGCUGAGCAAGUUUGG |
| | GGAGCGGAUUCCGGCCGAGAUGGCGCGCUUCUACCUGGCGGAGAUUG |
| | UCAUGGCCAUAGACUCGGUGCACCGGCUUGGCUACGUGCACAGGGAC |
| | AUCAAACCCGACAACAUCCUGCUGGACCGCUGUGGCCACAUCCGCCU |
| | GGCCGACUUCGGCUCUUGCCUCAAGCUGCGGGCAGAUGGAACGGUGC |
| | GGUCGCUGGUGGCUGUGGGCACCCCAGACUACCUGUCCCCCGAGAUC |
| | CUGCAGGCUGUGGGCGGUGGGCCUGGGACAGGCAGCUACGGGCCCGA |
| | GUGUGACUGGUGGGCGCUGGGUGUAUUCGCCUAUGAAAUGUUCUAU |
| | GGGCAGACGCCCUUCUACGCGGAUUCCACGGCGGAGACCUAUGGCAA |
| | GAUCGUCCACUACAAGGAGCACCUCUCUCUGCCGUGGUGGACGAAGG |
| | GGUCCCUGAGGAGGCUCGAGACUUCAUUCAGCGGUUGCUGUGUCCCC |
| | CGGAGACACGGCUGGGCCGGGGUGGAGCAGGCGACUUCCGGACACAU |
| | CCCUUCUUCUUUGGCCUCGACUGGGAUGGUCUCCGGGACAGCGUGCC |
| | CCCCUUUACACCGGAUUUCGAAGGUGCCACCGACACAUGCAACUUCG |
| | ACUUGGUGGAGGACGGGCUCACUGCCAUGGUGAGCGGGGGCGGGGAG |
| | ACACUGUCGGACAUUCGGGAAGGUGCGCCGCUAGGGGUCCACCUGCC |
| | UUUUGUGGGCUACUCCUACUCCUGCAUGGCCCUCAGGGACAGUGAGG |
| | UCCCAGGCCCCACACCCAUGGAACUGGAGGCCGAGCAGCUGCUUGAG |
| | CCACACGUGCAAGCGCCCAGCCUGGAGCCCUCGGUGUCCCCACAGGA |
| | UGAAACAGCUGAAGUGGCAGUUCCAGCGGCUGUCCCUGCGGCAGAGG |
| | CUGAGGCCGAGGUGACGCUGCGGGAGCUCCAGGAAGCCCUGGAGGAG |
| | GAGGUGCUCACCCGGCAGAGCCUGAGCCGGGAGAUGGAGGCCAUCCG |
| | CACGGACAACCAGAACUUCGCCAGUCAACUACGCGAGGCAGAGGCUC |
| | GGAACCGGGACCUAGAGGCACACGUCCGGCAGUUGCAGGAGCGGAUG |
| | GAGUUGCUGCAGGCAGAGGGAGCCACAGCUGUCACGGGGGUCCCCAG |
| | UCCCCGGGCCACGGAUCCACCUUCCCAUCUAGAUGGCCCCACGGCCGU |
| | GGCUGUGGGCCAGUGCCCGCUGGUGGGGCCAGGCCCCAUGCACCGCC |
| | GCCACCUGCUGCUCCCUGCCAGGGUCCCUAGGCCUGGCCUAUCGGAG |
| | GCGCUUUCCCUGCUCCUGUUCGCCGUUGUUCUGUCUCGUGCCGCCGC |
| | CCUGGGCUGCAUUGGGUUGGUGGCCCACGCCGGCCAACUCACCGCAG |
| | UCUGGCGCCGCCCAGGAGCCGCCCGCGCUCCCUGAACCCUAGAACUGU |
| | CUUCGACUCAGGGGCCCCGUUGGAAGACUGAGUGCCCGGGGCACGGC |
| | ACAGAAGCCGCGCCCACCGCCUGCCAGUUCACAACCGCUCCGAGCGUG |
| | GGUCUCCGCCCAGCACCAGUCCUGUGAUCCGGGCCCGCCCCCUAGCGG |
| | CCGGGGAGGGAGGGGCCGGGUCCGCGGCCGGCGAACGGGGCUCGAAG |
| | GGUCCUUGUAGCCGGGAAUGCUGCUGCUGCUGCUGGGGGGAUCACAG |
| | ACCAUUUCUUUCUUUCGGCCAGGCUGAGGCCCUGACGUGGAUGGGCA |
| | AACUGCAGGCCUGGGAAGGCAGCAAGCCGGGCCGUCCGUGUUCCAUC |
| | CUCCACGCACCCCCACCUAUCGUUGGUUCGCAAAGUGCAAAGCUUUC |
| | UUGUGCAUGACGCCCUGCUCUGGGGAGCGUCUGGCGCGAUCUCUGCC |
| | UGCUUACCCGGGAAAUUUGCUUUUGCCAAACCCGCUUUUUCGGGGAU |
| | CCCGCGCCCCCCUCCUCACUUGCGCUGCUCUCGGAGCCCCAGCCGGCU |
| | CCGCCCGCUUCGGCGGUUUGGAUAUUUAUUGACCUCGUCCUCCGACU |
| | CGCUGACAGGCUACAGGACCCCCAACAACCCCAAUCCACGUUUUGGA |
| | UGCACUGAGACCCCGACAUUCCUCGGUAUUUAUUGUCUGUCCCCACC |
| | UAGGACCCCCACCCCCGACCCUCGCGAAUAAAAGGCCCUCCAUCUGCC |
| | CAAAAAAAAAAAAAAAA |
| 8 | GGGACAGCCAGGGACAGGCAGACAUGCAGCCAGGGCUCCAGGGCCUG |
| | GACAGGGGCUGCCAGGCCCUGUGACAGGAGGACCCCGAGCCCCCGGC |
| | CCGGGGAGGGGCCAUGGUGCUGCCUGUCCAACAUGUCAGCCGAGGUG |
| | CGGCUGAGGCGGCUCCAGCAGCUGGUGUUGGACCCGGGCUUCCUGGG |
| | GCUGGAGCCCCUGCUCGACCUUCUCCUGGGCGUCCACCAGGAGCUGG |
| | GCGCCUCCGAACUGGCCCAGGACAAGUACGUGGCCGACUUCUUGCAG |
| | UGGGCGGAGCCCAUCGUGGUGAGGCUUAAGGAGGUCCGACUGCAGAG |
| | GGACGACUUCGAGAUUCUGAAGGUGAUCGGACGCGGGGCGUUCAGCG |
| | AGGUAGCGGUAGUGAAGAUGAAGCAGACGGGCCAGGUGUAUGCCAU |
| | GAAGAUCAUGAACAAGUGGGACAUGCUGAAGAGGGGCGAGGUGUCG |
| | UGCUUCCGUGAGGAGAGGGACGUGUUGGUGAAUGGGGACCGGCGGU |
| | GGAUCACGCAGCUGCACUUCGCCUUCCAGGAUGAGAACUACCUGUAC |
| | CUGGUCAUGGAGUAUUACGUGGGCGGGGACCUGCUGACACUGCUGAG |
| | CAAGUUUGGGGAGCGGAUUCCGGCCGAGAUGGCGCGCUUCUACCUGG |
| | CGGAGAUUGUCAUGGCCAUAGACUCGGUGCACCGGCUUGGCUACGUG |
| | CACAGGGACAUCAAACCCGACAACAUCCUGCUGGACCGCUGUGGCCA |
| | CAUCCGCCUGGCCGACUUCGGCCUUGCCUCAAGCUGCGGGCAGAUGG |
| | AACGGUGCGGUCGCUGGUGGCUGUGGGCACCCCAGACUACCUGUCCC |
| | CCGAGAUCCUGCAGGCUGUGGGCGGUGGGCCUGGGACAGGCAGCUAC |
| | GGGCCCGAGUGUGACUGGUGGGCGCUGGGUGUAUUCGCCUAUGAAAU |
| | GUUCUAUGGGCAGACGCCCUUCUACGCGGAUUCCACGGCGGAGACCU |
| | AUGGCAAGAUCGUCCACUACAAGGAGCACCUCUCUCUGCCGCUGGUG |
| | GACGAAGGGGUCCCUGAGGAGGCUCGAGACUUCAUUCAGCGGUUGCU |
| | GUGUCCCCCGGAGACACGGCUGGGCCGGGGUGGAGCAGGCGACUUCC |
| | GGACACAUCCCUUCUUCUUUGGCCUCGACUGGGAUGGUCUCCGGGAC |
| | AGCGUGCCCCCCUUUACACCGGAUUUCGAAGGUGCACCGACACAUGC |
| | AACUUCGACUUGGUGGAGGACGGGCUCACUGCCAUGGUGAGCGGGGG |
| | CGGGGAGACACUGUCGGACAUUCGGGAAGGUGCGCCGCUAGGGGUCC |
| | ACCUGCCUUUUGUGGGCUACUCCUACUCCUGCAUGGCCCUCAGGGAC |
| | AGUGAGGUCCCAGGCCCCACACCCAUGGAACUGGAGGCCGAGCAGCU |
| | GCUUGAGCCACACGUGCAAGCGCCCAGCCUGGAGCCCUCGGUGUCCC |
| | CACAGGAUGAAACAGCUGAAGUGGCAGUUCCAGCGGCUGUCCCUGCG |
| | GCAGAGGCUGAGGCCGAGGUGACGCUGCGGGAGCUCCAGGAAGCC |
| | CUGGAGGAGGAGGUGCUCACCCGGCAGAGCCUGAGCCGGGAGAUGGA |
| | GGCCAUCCGCACGGACAACCAGAACUUCGCCAGUCAACUACGCGAGG |
| | CAGAGGCUCGGAACCGGGACCUAGAGGCACACGUCCGGCAGUUGCAG |
| | GAGCGGAUGGAGUUGCUGCAGGCAGAGGGAGCCACAGCUGUCACGGG |
| | GGUCCCCAGUCCCCGGGCCACGGAUCCACCUUCCCAUCUAGAUGGCCC |
| | CCCGGCCGUGGCUGUGGGCCAGUGCCCGCUGGUGGGGCCAGGCCCCA |
| | UGCACCGCCGCCACCUGCUGCUCCCUGCCAGGGUCCCUAGGCCUGGCC |
| | UAUCGGAGGCGCUUUCCCUGCUCCUGUUCGCCGUUGUUCUGUCUCGU |
| | GCCGCCGCCCUGGGCUGCAUUGGGUUGGUGGCCCACGCCGGCCAACU |
| | CACCGCAGUCUGGCGCCGCCCAGGAGCCGCCCGCGCUCCCUGAACCCU |
| | AGAACUGUCUUCGACUCCGGGGCCCCGUUGGAAGACUGAGUGCCCGG |
| | GGCACGGCACAGAAGCCGCGCCCACCGCCUGCCAGUUCACAACCGCUC |
| | CGAGCGUGGGUCUCCGCCCAGCUCCAGUCCUGUGAUCCGGGCCCGCCC |
| | CCUAGCGGCCGGGGAGGGAGGGGCCGGGUCCGCGGCCGGCGAACGGG |
| | GCUCGAAGGGUCCUUGUAGCCGGGAAUGCUGCUGCUGCUGCUGGGGG |
| | GAUCACAGACCAUUUCUUUCUUUCGGCCAGGCUGAGGCCCUGACGUG |
| | GAUGGGCAAACUGCAGGCCUGGGAAGGCAGCAAGCCGGGCCGUCCGU |
| | GUUCCAUCCUCCACGCACCCCCACCUAUCGUUGGUUCGCAAAGUGCA |
| | AAGCUUUCUUGUGCAUGACGCCCUGCUCUGGGGAGCGUCUGGCGCGA |
| | UCUCUGCCUGCUUACUCGGGAAAUUUGCUUUUGCCAAACCCGCUUUU |
| | UCGGGGAUCCCGCGCCCCCCUCCUCACUUGCGCUGCUCUCGGAGCCCC |
| | AGCCGGCUCCGCCCGCUUCGGCGGUUUGGAUAUUUAUUGACCUCGUC |
| | CUCCGACUCGCUGACAGGCUACAGGACCCCCAACAACCCCAAUCCACG |
| | UUUUGGAUGCACUGAGACCCCGACAUUCCUCGGUAUUUAUUGUCUGU |
| | CCCCACCUAGGACCCCCACCCCCGACCCUCGCGAAUAAAAGGCCCUCC |
| | AUCUGCCCAAAAAAAAAAAAAAAAAAAAAAAAAAAAA |

The terms “dystrophia myotonica protein kinase” and “DMPK” as used herein include, for example, forms of the human DMPK transcript that have a nucleic acid sequence that is at least 85% identical to the nucleic acid sequence of SEQ ID NO: 7 or SEQ ID NO: 8 (e.g., 85%, 86%, 87%, 88%, 89%, 90%, 91%, 92%, 93%, 94%, 95%, 96%, 97%, 98%, 99%, 99.9%, or 100% identical to the nucleic acid sequence of SEQ ID NO: 7 or SEQ ID NO: 8 and/or forms of the human DMPK gene that encode a DMPK protein having one or more (e.g., up to 25) conservative amino acid substitutions relative to a wild-type DMPK protein. The terms “dystrophia myotonica protein kinase” and “DMPK” as used herein additionally include DMPK RNA transcripts containing expanded CUG trinucleotide repeat regions relative to the length of the CUG trinucleotide repeat region of a wild-type DMPK mRNA transcript. The expanded repeat region may contain, for example, 50 or more CUG trinucleotide repeats, such as from about 50 to about 4,000 CUG trinucleotide repeats (e.g., about 50 CUG trinucleotide repeats, about 60 CUG trinucleotide repeats, about 70 trinucleotide repeats, 80 trinucleotide repeats, 90 trinucleotide repeats, 100 trinucleotide repeats, 110 trinucleotide repeats, 120 trinucleotide repeats, 130 trinucleotide repeats, 140 trinucleotide repeats, 150 trinucleotide repeats, 160 trinucleotide repeats, 170 trinucleotide repeats, 180 trinucleotide repeats, 190 trinucleotide repeats, 200 trinucleotide repeats, 210 trinucleotide repeats, 220 trinucleotide repeats, 230 trinucleotide repeats, 240 trinucleotide repeats, 250 trinucleotide repeats, 260 trinucleotide repeats, 270 trinucleotide repeats, 280 trinucleotide repeats, 290 trinucleotide repeats, 300 trinucleotide repeats, 310 trinucleotide repeats, 320 trinucleotide repeats, 330 trinucleotide repeats, 340 trinucleotide repeats, 350 trinucleotide repeats, 360 trinucleotide repeats, 370 trinucleotide repeats, 380 trinucleotide repeats, 390 trinucleotide repeats, 400 trinucleotide repeats, 410 trinucleotide repeats, 420 trinucleotide repeats, 430 trinucleotide repeats, 440 trinucleotide repeats, 450 trinucleotide repeats, 460 trinucleotide repeats, 470 trinucleotide repeats, 480 trinucleotide repeats, 490 trinucleotide repeats, 500 trinucleotide repeats, 510 trinucleotide repeats, 520 trinucleotide repeats, 530 trinucleotide repeats, 540 trinucleotide repeats, 550 trinucleotide repeats, 560 trinucleotide repeats, 570 trinucleotide repeats, 580 trinucleotide repeats, 590 trinucleotide repeats, 600 trinucleotide repeats, 610 trinucleotide repeats, 620 trinucleotide repeats, 630 trinucleotide repeats, 640 trinucleotide repeats, 650 trinucleotide repeats, 660 trinucleotide repeats, 670 trinucleotide repeats, 680 trinucleotide repeats, 690 trinucleotide repeats, 700 trinucleotide repeats, 710 trinucleotide repeats, 720 trinucleotide repeats, 730 trinucleotide repeats, 740 trinucleotide repeats, 750 trinucleotide repeats, 760 trinucleotide repeats, 770 trinucleotide repeats, 780 trinucleotide repeats, 790 trinucleotide repeats, 800 trinucleotide repeats, 810 trinucleotide repeats, 820 trinucleotide repeats, 830 trinucleotide repeats, 840 trinucleotide repeats, 850 trinucleotide repeats, 860 trinucleotide repeats, 870 trinucleotide repeats, 880 trinucleotide repeats, 890 trinucleotide repeats, 900 trinucleotide repeats, 910 trinucleotide repeats, 920 trinucleotide repeats, 930 trinucleotide repeats, 940 trinucleotide repeats, 950 trinucleotide repeats, 960 trinucleotide repeats, 970 trinucleotide repeats, 980 trinucleotide repeats, 990 trinucleotide repeats, 1,000 trinucleotide repeats, 1,100 trinucleotide repeats, 1,200 trinucleotide repeats, 1,300 trinucleotide repeats, 1,400 trinucleotide repeats, 1,500 trinucleotide repeats, 1,600 trinucleotide repeats, 1,700 trinucleotide repeats, 1,800 trinucleotide repeats, 1,900 trinucleotide repeats, 2,000 trinucleotide repeats, 2,100 trinucleotide repeats, 2,200 trinucleotide repeats, 2,300 trinucleotide repeats, 2,400 trinucleotide repeats, 2,500 trinucleotide repeats, 2,600 trinucleotide repeats, 2,700 trinucleotide repeats, 2,800 trinucleotide repeats, 2,900 trinucleotide repeats, 3,000 trinucleotide repeats, 3,100 trinucleotide repeats, 3,200 trinucleotide repeats, 3,300 trinucleotide repeats, 3,400 trinucleotide repeats, 3,500 trinucleotide repeats, 3,600 trinucleotide repeats, 3,700 trinucleotide repeats, 3,800 trinucleotide repeats, 3,900 trinucleotide repeats, or 4,000 trinucleotide repeats, among others).

As used herein, the term “interfering RNA” refers to a RNA, such as a short interfering RNA (siRNA), micro RNA (miRNA), or short hairpin RNA (shRNA) that suppresses the expression of a target RNA transcript by way of (i) annealing to the target RNA transcript, thereby forming a nucleic acid duplex; and (ii) promoting the nuclease-mediated degradation of the RNA transcript and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient, such as a human patient having myotonic dystrophy, in the form of, for example, a single- or double-stranded oligonucleotide, or in the form of a vector (e.g., a viral vector, such as an adeno-associated viral vector described herein) containing a transgene encoding the interfering RNA. Exemplary interfering RNA platforms are described, for example, in Lam et al., Molecular Therapy-Nucleic Acids 4: e252 (2015); Rao et al., Advanced Drug Delivery Reviews 61:746-769 (2009); and Borel et al., Molecular Therapy 22:692-701 (2014), the disclosures of each of which are incorporated herein by reference in their entirety. As used herein, the “length” of a nucleic acid refers to the linear size of the nucleic acid as assessed by measuring the quantity of nucleotides from the 5′ to the 3′ end of the nucleic acid. Exemplary molecular biology techniques that may be used to determine the length of a nucleic acid of interest are known in the art.

As used herein, the term “myotonic dystrophy” refers to an inherited muscle wasting disorder characterized by the nuclear retention of RNA transcripts encoding DMPK and containing an expanded CUG trinucleotide repeat region in the 3′ untranslated region (UTR), such as an expanded CUG trinucleotide repeat region having from 50 to 4,000 CUG repeats. Wild-type RMPK RNA transcripts, by comparison, typically contain from 5 to 37 CUG repeats in the 3′ UTR. In patients having myotonic dystrophy, the expanded CUG repeat region interacts with RNA-binding splicing factors, such as muscleblind-like protein. This interaction causes the mutant transcript to be retained in nuclear foci and leads to sequestration of RNA-binding proteins away from other pre-mRNA substrates, which, in turn, promotes spliceopathy of proteins involved in modulating muscle structure and function. In type I myotonic dystrophy (DM1), skeletal muscle is often the most severely affected tissue, but the disease also imparts toxic effects on cardiac and smooth muscle, the ocular lens, and the brain. The cranial, distal limb, and diaphragm muscles are preferentially affected. Manual dexterity is compromised early, which causes several decades of severe disability. The median age at death of myotonic dystrophy patients is 55 years, which is usually caused by respiratory failure (de Die-Smulders C E, et al., Brain 121:1557-1563 (1998)).

TABLE 4
MBNL transcript variants

| SEQ ID NO: | Nucleic Acid Sequence |
|---|---|
| 9 | agtcttttca ctgcagctga atgagttgtg gcgcccacaa tgctcccatg acaaggagct |
| | gacaagttcc attttccgtc gcgggcatct tggaatcatg actcccacaa tgccttgggc |
| | acttggtcga cagtggggcc gcctctgaaa aaaaaatgtg agaggttggt actaagaagt |
| | gcctttcctg acgtctctgc tgcttggaac cgcttctaga gcagtctctg cttttgcctt |
| | gcttgctgcc agctagactg tgacgacagc acatccaccc tccacctcta gcccagacac |
| | ccccatttct acttataatc aagagaaaag ctctaagtat ctggcattgc cctaggctgc |
| | tttagtgtta aaagaaaagt ttgctgaaaa agtaagatat cttctgccag gaaatcaagg |
| | aggaaaaaaa aaatcatttt ctcgattttg ctctaaactg ctgcatctgt ctatgccaaa |
| | ctaatcaata ccgattgcac caccaaactc cattgcaaat tcagctgtga ggagattccc |
| | tttcagacaa ctttgctgaa agcagcttgg aaattcggtg tcgaagggtc tgccacgttt |
| | tcatgcttgc attttgggct ccaaattggc actgggaagg ggttactgag agcacaaggc |
| | tgataccagg ccctactttt aaacgttcat ctacttacaa tcctagtatt tctctaaaaa |
| | ccaaaacctc tttgaattaa cagtttcatg ctgtgaattt ctagtgggag atcttttcct |
| | tgatattgac gacacaattt tccatgtact tttaaagcag ggagtgggga aaagtatttt |
| | gaggggacat tttcatcatc agttcagctt tttttttttg gttgttgctc ttttttgggg |
| | gggttgggtt tgttggtttc actgaaacat ttaactacct gtaaaatcta aacatggctg |
| | ttagtgtcac accaattcgg gacacaaaat ggctaacact ggaagtatgt agagagttcc |
| | agagggggac ttgctcacgg ccagacacgg aatgtaaatt tgcacatcct tcgaaaagct |
| | gccaagttga aaatggacga gtaatcgcct gctttgattc attgaaaggc cgttgctcca |
| | gggagaactg caaatatctt catccacccc cacatttaaa aacgcagttg gagataaatg |
| | gacgcaataa cttgattcag cagaagaaca tggccatgtt ggcccagcaa atgcaactag |
| | ccaatgccat gatgcctggt gccccattac aacccgtgcc aatgttttca gttgcaccaa |
| | gcttagccac caatgcatca gcagccgcct ttaatcccta tctgggacct gtttctccaa |
| | gcctggtccc ggcagagatc ttgccgactg caccaatgtt ggttacaggg aatccgggtg |
| | tccctgtacc tgcagctgct gcagctgctg cacagaaatt aatgcgaaca gacagacttg |
| | aggtatgtcg agagtaccaa cgtggcaatt gcaaccgagg agaaaatgat tgtcggtttg |
| | ctcatcctgc tgacagcaca atgattgaca ccaatgacaa cacagtcact gtgtgtatgg |
| | attacatcaa agggagatgc tctcgggaaa agtgcaaata ctttcatccc cctgcacatt |
| | tgcaagccaa gatcaaggct gcccaatacc aggtcaacca ggctgcagct gcacaggctg |
| | cagccaccgc agctgccatg ggaattcctc aagctgtact tcccccatta ccaaagaggc |
| | ctgctcttga aaaaaccaac ggtgccaccg cagtctttaa cactggtatt ttccaatacc |
| | aacaggctct agccaacatg cagttacaac agcatacagc atttctccca ccaggctcaa |
| | tattgtgcat gacacccgct acaagtgttg ttcccatggt gcacggtgct acgccagcca |
| | ctgtgtccgc agcaacaaca tctgccacaa gtgttccctt cgctgcaaca gccacagcca |
| | accagatacc cataatatct gccgaacatc tgactagcca caagtatgtt acccagatgt |
| | agaattttca tcactaaaca atcatgctaa agaggaaagg acagtgtgct tggttagagt |
| | aaaggacgag gtcattagcc atattgtata tatcgtcaag caacacacac aaaagttcct |
| | cagccacaag acatccacat attgcatgtt aaccagaaga aaagacaaca ttttccggaa |
| | atccactgca cactgttgcc tatacacttt gtacatttaa ttgatatttg tgctgaggtg |
| | atattcctgt ctaaaagaac aacattgtct ttcttttcta gcacagagtt atgcattcaa |
| | agatgcatac ctagttagtt tcctatatat tcatgccatc ttgaaaagac agactatggt |
| | gtaaccatga ttctattatg tattggtacg tctgtagacc aagatataat tttttaaaaa |
| | taagtttatt tctttcaagg tttacaaata acaaaggtgc accttgtatt taaaattgcc |
| | attatagatg agagcgtgca tgcacagtca tttttgttta agagtaatat ttttaatgta |
| | atagattgta agacgtggtg agggagggat ctgacagaga tgaatgtgcc aagcaaaacc |
| | acaactgtgt atattttaaa gcacatcatg gctttaagta ccatgttgtt aaggattctc |
| | atgaagtgcc atagactgta catcaaatta gagtattatt tcttcagtgt tattgttttc |
| | agagccacat tttgttgcat atttgctagt actaatcagt caaagggcac cattcttttt |
| | tttttttttt gaaaccaaag ctgtctcaga aatggccaat ttaactttac agtaacaata |
| | gacagcacaa cacaaactct ctcaatacag ataaactcac acatactgga gatatatata |
| | taatagatat atataaaatt attttaatgc attgtagtgt aatatttatg catactatac |
| | tgtataacat gttattcaaa agggattgcc atttctgaga cacagtaaca aaaaaatgag |
| | gaaattattt tgcttctatt tatagcctct gtcaaaagtc aaaagactat aaatgctttg |
| | caaaaatggt ttcacgtttg cttaaatgct tcatcacagt cacattcaaa atagtgactc |
| | taaacaaaga agaaagcagc actgtcatca gatgcatgat aaaccaaaat atgaaaatgg |
| | gaaatgttta attaacctag taattgggtg ggttaagtac atgggtgaat tttatatgtg |
| | atttttgttt tgttttgttt tgttcagatt aactgcttat agccttagaa agccttttac |
| | aaaattaaaa aaaaaataga tgtgcattca gtttttaaga atggaatcat ccaaaggaat |
| | tccttttttt gaggtttgga tgttgcagct agtaaaggat atttttgctc tgttcagcag |
| | ttctaaaaat tgctgaagta ggggccaggt cactggtagt tatagtatgg aatgggagaa |
| | gtgaaagttc agttatagaa ctttccatac ttccaagttt actgcaagtt tttatgcttg |
| | agagagatgc tttctaatat aagactgatg tgttgatttt actgattgta ctgtacatct |
| | attaaagcct tagattatta cattacgggt tggaacccat accaatgtaa tttcaatcgt |
| | gttaagaaag taatggtgac ttcacatgtt attgtagtta gttacattat agaatattac |
| | ttatttttct tgttaaaatg tagtttttca tttcctacat ttattagatt ttcattttct |
| | attaacaatt gaataccatt tcagtttata gacttgtttt attagatttt accaatgaat |
| | ttttcaaaat acaaaaaaaa gtagtttttc cttcataaca tactcagttt tgaattacat |
| | gtagtgtcac atgaatattc gtattgttaa ctaaatgatt tatattttac tgatttaata |
| | ttacagtgta agaatgtcag tcattgttag ttcttgtcta gttttcatta aaagaacaaa |
| | gatcttttat atggatatct tataaatata taatcattgc taagtaagaa gttaagttgt |
| | tgctatcgca acaatcctgg cagacaattg agtaatattt tgatgattta ttttgtttgt |
| | aattagttat tataagaaga tctagatcct agatattaga ataaaattta ttttctactg |
| | tatccatttc aaatgttaaa atattgttta atatttttga aatccctgag tatcaggcct |
| | tgttataaat aagctgcata atcaataaat agaacaaggg actttttgtt gataatccaa |
| | atactcaaag tttacgtaat gaaaattata gcgtgtgtgc aaactcttga gggttgatta |
| | tgctgcaatt tagcatgttg gaacgtctag ggagaaggtt gactttttgc acttctgtat |
| | atagtcaaaa gagagaaacc tgtataatag taagatctta ttttgaataa aaacgtctat |
| | aattacaagg agttttgtta aggctaatac aatgacagac tgagcaaaat tgcttgcaaa |
| | agtggcacag agttagcact ccatacccct tcaaacatgt tgctttgctt tcttgtggac |
| | agcttgtagt ttgccaggat tttttcagct ggaaagatac gccatccttt caaaccctca |
| | tgactgacaa aaactccatg gggccaaatc tgcctgaaga tcattaccaa aaatagcagg |
| | tacttctacc attaaggtga aatcatggat cagatattcc ttacattttt caaaactact |
| | gcatgtttaa aacttcaaca aaaaaagaga gaaagaacta tactaagaac atatattatt |
| | cagatcagtt tctgccaatt tcagtggttt attgttcaca aaaaaatctt caaaacaagt |
| | attgactttc acaaaattta aatcataaac aggcaaacca aacagcacac tgtagctata |
| | gttgttatgt gattgttttt taattgctgt aggatcctgt tctttcagca ggtgaaaaat |
| | aaaacgcagt tcaaatttca tggttttaat tttcaactca gaagcactca aaaatgcaaa |
| | atgtgataat gggcacttgt ttaaaagaat tagtgtatcc agccttcact ccagctggtt |
| | aaaaatgttg cacttatcag caaccctacc actttcatct gctgaaagga caaatgtgct |
| | tggttttact attatgtaat cacaacttac tttctgcttg tagttgctta aaattatgta |
| | ttttgtcttg ggctgcaatt tgttttatgc ttattttatt attactgcag tagttgactt |
| | tgctgtatgg aaaaataaag tgaaattgcc ctaataaaac ttctctttct taagta |
| 10 | agactgtctt tgcttcatca tctgaaggta aaattttcca gatacggcag acggctttca |
| | gagtacaata aacagggaat gagaactatt tacatggaag tttctttctc atgatgcggt |
| | ggagaagcct cggccacttg gttctgccag atgttcctgg ggttactgta aatgggaagg |
| | acaggcagag ctaaacaagg tttatcattt aaaagtgcct gtgtgaagtc acttttgctg |
| | gaaaactgca gcttgggagc tttctttgta ttcacatccc actcttctgt caagtacact |
| | ttaccctgac cttatgagtg gatgaagata cctcagttgt ctgactttgc caattgctta |
| | atttcagaat ttaaaaaggg gaaagaaaaa catcctgcta aaatatgaac atctgagtgt |
| | cttattttcc aacatcgtca atagctgtga gcgtcagcat taaatattct cccaaggagt |
| | gccatgatat tgaagtcact ttattaataa cagctgtatc tgcaaaacag tcaagagact |
| | cggacgttga aagccagaga tgacactgag catgctttta ttgcggccta ccatctttaa |
| | gtgggacata ttgattgatg agtgattgcc tgtccataca ctctctcatc atcctgttcc |
| | ttggattgga cttcactaag caatttatca ctcaccttca gacttacatg tgggagtttt |
| | cacaacagta gttttggaat cattagaact tggattgatt tcatcattta acagaaacaa |
| | acagcccaaa ttactttatc accatggctt tgaacgttgc cccagtcaga gatacaaaat |
| | ggctgacatt agaagtctgc agacagtttc aaagaggaac atgctcacgc tctgatgaag |
| | aatgcaaatt tgctcatccc cccaaaagtt gtcaggttga aaatggaaga gtaattgcct |
| | gctttgattc cctaaagggc cgttgttcga gagagaactg caagtatctt caccctccga |
| | cacacttaaa aactcaacta gaaattaatg gaaggaacaa tttgattcag caaaaaactg |
| | cagcagcaat gcttgcccag cagatgcaat ttatgtttcc aggaacacca cttcatccag |
| | tgcccacttt ccctgtaggt cccgcgatag ggacaaatac ggctattagc tttgctcctt |
| | acctagcacc tgtaacccct ggagttgggt tggtcccaac ggaaattctg cccaccacgc |
| | ctgttattgt tcccggaagt ccaccggtca ctgtcccggg ctcaactgca actcagaaac |
| | ttctcaggac tgacaaactg gaggtatgca gggagttcca gcgaggaaac tgtgcccggg |
| | gagagaccga ctgccgcttt gcacaccccg cagacagcac catgatcgac acaagtgaca |
| | acaccgtaac cgtttgtatg gattacataa aggggcgttg catgagggag aaatgcaaat |
| | attttcaccc tcctgcacac ttgcaggcca aaatcaaagc tgcgcagcac caagccaacc |
| | aagctgcggt ggccgcccag gcagccgcgg ccgcggccac agtcatgact |
| | cagtcgactg ccaaagcaat gaagcgacct ctcgaagcaa ctgtagacct |
| | ggcctttccc cctggtgctc ttcatccttt accaaagaga caagcacttg aaaaaagcaa |
| | tggtaccagc gcggtcttta accccagcgt cttgcactac cagcaggctc tcaccagcgc |
| | acagttgcag caacacgccg cgttcattcc aacagggtca gttttgtgca tgacacccgc |
| | taccagtatt gataattctg aaataatcag cagaaacgga atggaatgcc aagaatctgc |
| | attgagaata actaaacatt gttactgtac atactatcct gtttcctcct caatagaatt |
| | gccacaaact gcatgctaaa taaagatgta gttcttctgg acagaccaca actctaagaa |
| | gctagtgctg ctatctcata tatgagtatt aaatatggta tgcttagtat attccaacct |
| | aagatagtta actacctgag accagctgtg atgtttaaag acataaagga taaagtttac |
| | ttttaaaggg tttctaaaca tagtttctgt cctaggaata ttgtcttatc tccataacta |
| | tagctgatgc agaaagtcca gccagtttac tcatttcgat tcagaatatt tcaaatttag |
| | caataaacaa ttagcattag ttaaaaaaga aacatattcc aagggcaggt tcgattctag |
| | ctctaattac tgtcatgtca tttacccact ggatcaaagg gtatgtttca cttcttgaca |
| | atataaatgc tgcagcaaag atgagaggtg aagtaaaacc gatacctgtc ctgcaggtct |
| | aaaatttgaa tggaaattca agcacaagta ctggggacac atcaaagtgt ggtgtttggt |
| | ttgcctggag atgccacgtt gaatcatgtg attctagatt aacattaaat agattgaaaa |
| | agaaactttg cacggtatga gcttcatacc ccaccaaaca aagtcttgaa ggtattattt |
| | tacaagtata tttttaaagt tgttttataa gagagacttt gtagaagtgc ctagattttg |
| | ccagacttca tccagcttga caagattgag aggcccatgc caacagtcta atctaagaga |
| | ttagtctttc aaactcacca tccagttgcc tgttacagaa taactcttct taactaaaaa |
| | cctagtcaaa caaggaagct gtaggtgagg agatctgtat aatattctaa tttaagtaag |
| | tttgagttta gtcactgcaa atttgactgt gactttaatc taaattacta tgtaaacaaa |
| | aagtagatag tttcactttt taaaaaatcc attactgttt tgcatttcaa aagttggatt |
| | aaagggttgt aactgactac agcatggaaa aaaatagttc ttttaattct ttcaccttaa |
| | agcatatttt atgtctcaaa agtataaaaa actttaatac aagtacatac atattatata |
| | tacacataca tatatatact atatatggat gaaacatatt ttaatgttgt ttactttttt |
| | aaatacttgg ttgatcttca aggtaatagc gatacaatta aattttgttc agaaagtttg |
| | ttttaaagtt tattttaagc actatcgtac caaatatttc atatttcaca ttttatatgt |
| | tgcacatagc ctatacagta cctacatagt ttttaaatta ttgtttaaaa aacaaaacag |
| | ctgttataaa tgaatattat gtgtaattgt ttcaaacatc cattttcttt gtgaacatat |
| | tagtgattga agtattttga cttttgagat tgaatgtaaa atattttaaa tttgggatca |
| | tcgcctgttc tgaaaactag atgcaccaac cgtatcatta tttgtttgag gaaaaaaaga |
| | aatctgcatt ttaattcatg ttggtcaaag tcgaattact atctatttat cttatatcgt |
| | agatctgata accctatcta aaagaaagtc acacgctaaa tgtattctta catagtgctt |
| | gtatcgttgc atttgtttta atttgtggaa aagtattgta tctaacttgt attactttgg |
| | tagtttcatc tttatgtatt attgatattt gtaattttct caactataac aatgtagtta |
| | cgctacaact tgcctaaaac attcaaactt gttttctttt ttctgttttt ttctttgtta |
| | attcatttaa actcattgaa aacatagtat acattactaa aaggtaaatt atgggaatca |
| | ctgaaatatt tttgtagatt aattgttgta acattgtctt tctttttttt cttttgtttc atgattttga |
| | tttttaaaat tattagcaca caactatttt cagcccttta ataatggagc atcaaaaaca |
| | tcacctgtaa ccccaagcaa atatagaaga ctgtattttt tactatgata tccattttcc |
| | agaattgtga ttacaatatg caaagagtca taaatatgcc atttacaata aggaggaggc |
| | aaggcaaatg catagatgta caaatatatg tacaacagat tttgcttttt atttatttat |
| | aatgtaattt tatagaataa ttctgggatt tgagaggatc taaaactatt tttctgtata |
| | aatattattt gccaaaagtt tgtttatatt cagaagtctg actatgatga ataaatctta |
| | aatgctttgt ttaattaaaa aacaaaaatc accaatatcc aagacatgaa gatatcagtt |
| | caacaaatac tgtagttaag agactaactc tccacttgta tgggaactac atttcactct |
| | tggttttcag gatataacag cacttcaccg aaatattctt tcagccatac cactggtaac |
| | atttctacta aatctttctg taacacttaa agaattccct cattcattac cttacagtgt |
| | aaacaggagt ctaatttgta tcaatactat gttttggttg taatattcag ttcactcacc |
| | caatgtacaa ccaatgaaat aaaagaagca tttaaaagga |

As used herein, the term “operably linked” refers to a first molecule (e.g., a first nucleic acid) joined to a second molecule (e.g., a second nucleic acid), wherein the molecules are so arranged that the first molecule affects the function of the second molecule. The two molecules may or may not be part of a single contiguous molecule and may or may not be adjacent to one another. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, one segment of a nucleic acid molecule is considered to “overlap with” another segment of the same nucleic acid molecule if the two segments share one or more constituent nucleotides. For example, two segments of the same nucleic acid molecule are considered to “overlap with” one another if the two segments share 1, 2, 3, 4, 5, 6, 7, 8, 9, 10, 15, 20, 25, 30, 35, 40, 45, 50, 100, or more, constituent nucleotides. The two segments are not considered to “overlap with” one another if the two segments have zero constituent nucleotides in common.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:
100 multiplied by (the fraction X/Y)
where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

As used herein, the term “functional MBNL” or “functional MBNL1” refers to MBNL protein that is not bound to a CTG microsatellite repeat in the 3′ UTR of a nucleic acid encoding DMPK.

As used herein, the term “pharmaceutical composition” refers to a mixture containing a therapeutic agent, such as a nucleic acid construct or expression vector described herein, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions, and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “repeat region” refers to segments within a gene of interest or an RNA transcript thereof containing nucleic acid repeats, such as the poly CTG sequence in the 3″ UTR of the human DMPK gene (or the poly CUG sequence in the 3′ UTR of the RNA transcript thereof). A repeat region is considered to be an “expanded repeat region.” a “repeat expansion,” or the like, if the number of nucleotide repeats in the repeat region exceeds the quantity of repeats ordinarily found in the repeat region of a wild-type form of the gene or RNA transcript thereof. For example, the 3′ UTRs of wild-type human DMPK genes typically contain from 5 to 37 CTG or CUG repeats. “Expanded repeat regions” and “repeat expansions” in the context of the DMPK gene or an RNA transcript thereof thus refer to repeat regions containing greater than 37 CTG or CUG repeats, such as from about 50 to about 4,000 CUG trinucleotide repeats (e.g., about 50 CUG trinucleotide repeats, about 60 CUG trinucleotide repeats, about 70 trinucleotide repeats, 80 trinucleotide repeats, 90 trinucleotide repeats, 100 trinucleotide repeats, 110 trinucleotide repeats, 120 trinucleotide repeats, 130 trinucleotide repeats, 140 trinucleotide repeats, 150 trinucleotide repeats, 160 trinucleotide repeats, 170 trinucleotide repeats, 180 trinucleotide repeats, 190 trinucleotide repeats, 200 trinucleotide repeats, 210 trinucleotide repeats, 220 trinucleotide repeats, 230 trinucleotide repeats, 240 trinucleotide repeats, 250 trinucleotide repeats, 260 trinucleotide repeats, 270 trinucleotide repeats, 280 trinucleotide repeats, 290 trinucleotide repeats, 300 trinucleotide repeats, 310 trinucleotide repeats, 320 trinucleotide repeats, 330 trinucleotide repeats, 340 trinucleotide repeats, 350 trinucleotide repeats, 360 trinucleotide repeats, 370 trinucleotide repeats, 380 trinucleotide repeats, 390 trinucleotide repeats, 400 trinucleotide repeats, 410 trinucleotide repeats, 420 trinucleotide repeats, 430 trinucleotide repeats, 440 trinucleotide repeats, 450 trinucleotide repeats, 460 trinucleotide repeats, 470 trinucleotide repeats, 480 trinucleotide repeats, 490 trinucleotide repeats, 500 trinucleotide repeats, 510 trinucleotide repeats, 520 trinucleotide repeats, 530 trinucleotide repeats, 540 trinucleotide repeats, 550 trinucleotide repeats, 560 trinucleotide repeats, 570 trinucleotide repeats, 580 trinucleotide repeats, 590 trinucleotide repeats, 600 trinucleotide repeats, 610 trinucleotide repeats, 620 trinucleotide repeats, 630 trinucleotide repeats, 640 trinucleotide repeats, 650 trinucleotide repeats, 660 trinucleotide repeats, 670 trinucleotide repeats, 680 trinucleotide repeats, 690 trinucleotide repeats, 700 trinucleotide repeats, 710 trinucleotide repeats, 720 trinucleotide repeats, 730 trinucleotide repeats, 740 trinucleotide repeats, 750 trinucleotide repeats, 760 trinucleotide repeats, 770 trinucleotide repeats, 780 trinucleotide repeats, 790 trinucleotide repeats, 800 trinucleotide repeats, 810 trinucleotide repeats, 820 trinucleotide repeats, 830 trinucleotide repeats, 840 trinucleotide repeats, 850 trinucleotide repeats, 860 trinucleotide repeats, 870 trinucleotide repeats, 880 trinucleotide repeats, 890 trinucleotide repeats, 900 trinucleotide repeats, 910 trinucleotide repeats, 920 trinucleotide repeats, 930 trinucleotide repeats, 940 trinucleotide repeats, 950 trinucleotide repeats, 960 trinucleotide repeats, 970 trinucleotide repeats, 980 trinucleotide repeats, 990 trinucleotide repeats, 1,000 trinucleotide repeats, 1,100 trinucleotide repeats, 1,200 trinucleotide repeats, 1,300 trinucleotide repeats, 1,400 trinucleotide repeats, 1,500 trinucleotide repeats, 1,600 trinucleotide repeats, 1,700 trinucleotide repeats, 1,800 trinucleotide repeats, 1,900 trinucleotide repeats, 2,000 trinucleotide repeats, 2,100 trinucleotide repeats, 2,200 trinucleotide repeats, 2,300 trinucleotide repeats, 2,400 trinucleotide repeats, 2,500 trinucleotide repeats, 2,600 trinucleotide repeats, 2,700 trinucleotide repeats, 2,800 trinucleotide repeats, 2,900 trinucleotide repeats, 3,000 trinucleotide repeats, 3,100 trinucleotide repeats, 3,200 trinucleotide repeats, 3,300 trinucleotide repeats, 3,400 trinucleotide repeats, 3,500 trinucleotide repeats, 3,600 trinucleotide repeats, 3,700 trinucleotide repeats, 3,800 trinucleotide repeats 3,900 trinucleotide repeats, or 4,000 trinucleotide repeats, among others).

As used herein, the phrases “specifically binds” and “binds” refer to a binding reaction which is determinative of the presence of a particular molecule, such as an RNA transcript, in a heterogeneous population of ions, salts, small molecules, and/or proteins that is recognized, e.g., by a ligand or receptor, such as an RNA-binding splicing factor protein, with particularity. A ligand (e.g., an RNA-binding protein described herein) that specifically binds to a species (e.g., an RNA transcript) may bind to the species, e.g., with a K_{D} of less than 1 mM. For example, a ligand that specifically binds to a species may bind to the species with a K_{D} of up to 100 μM (e.g., between 1 μM and 100 μM). A ligand that does not exhibit specific binding to another molecule may exhibit a K_{D} of greater than 1 mM (e.g., 1 μM, 100 μM, 500 μM, 1 mM, or greater) for that particular molecule or ion. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” refer to an organism that receives treatment for a particular disease or condition as described herein (such as a heritable muscle-wasting disorder, e.g., myotonic dystrophy). Examples of subjects and patients include mammals, such as humans, receiving treatment for a disease or condition described herein.

As used herein, the terms “treat” or “treatment” refer to therapeutic treatment, in which the object is to prevent or slow down (lessen) an undesired physiological change or disorder, such as the progression of a heritable muscle-wasting disorder, for example, myotonic dystrophy, and particularly, type I myotonic dystrophy. In the context of myotonic dystrophy treatment, beneficial or desired clinical results that are indicative of successful treatment include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment of a patient having myotonic dystrophy (e.g., type I myotonic dystrophy may manifest in one or more detectable changes, such as a decrease in the expression of DMPK RNA transcripts that contain expanded CUG trinucleotide repeat regions (e.g., a decrease in the expression of DMPK RNA transcripts that contain expanded CUG trinucleotide repeat regions of 1% or more, such as a decrease of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%, relative to the expression of DMPK RNA transcripts containing expanded CUG trinucleotide repeat regions by the patient prior to administration of a therapeutic agent, such as a vector or nucleic acid described herein. Methods that can be used to assess RNA expression levels are known in the art and include RNA-seq assays and polymerase chain reaction techniques described herein. Additional clinical indications of successful treatment of a CPVT patient include alleviation of spliceopathy, for example, of an RNA transcript that is spliced in a manner that is dependent upon muscleblind-like protein. For example, observations that signal successful treatment of a patient having myotonic dystrophy include a finding that the patient exhibits an increase in corrective splicing of one or more RNA transcript substrates of muscleblind-like protein following administration of a therapeutic agent, such as a therapeutic agent described herein. For example, indicators that signal successful treatment of myotonic dystrophy include a determination that the patient exhibits an increase in expression of sarcoplasmic/endoplasmic reticulum calcium ATPase 1 (SERCA1) mRNA containing exon 22, such as an increase of about 1.1-fold to about 10-fold, or more (e.g., an increase in expression of SERCA1 mRNA containing exon 22 by about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, or more), as assessed, for example, using an RNA or protein detection assay described herein. Treatment of myotonic dystrophy may also manifest as a decrease in expression of chloride voltage-gated channel 1 (CLCN1) mRNA containing exon 7a, such as a decrease of about 1% to about 100% (e.g., a decrease in expression of CLCN1 mRNA containing exon 7a by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%), as assessed, for example, using an RNA or protein detection assay described herein. Additionally, successful treatment may be signaled by a determination that the patient exhibits a decrease in expression of ZO-2 associated speckle protein (ZASP) containing exon 11, such as a decrease of about 1% to about 100% (e.g., a decrease in expression of ZASP mRNA containing exon 11 by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%), as assessed, for example, using an RNA or protein detection assay described herein. Successful treatment of myotonic dystrophy may also be signaled by a finding that, following the therapy, the patient exhibits an increase in corrective splicing of RNA transcripts encoding insulin receptor, ryanodine receptor 1 (RYR1), cardiac muscle troponin, and/or skeletal muscle troponin, such as an increase of about 1.1-fold to about 10-fold, or more (e.g., an increase in expression of correctly spliced RNA transcripts encoding insulin receptor, RYR1, cardiac muscle troponin, and/or skeletal muscle troponin by about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, or more), as assessed, for example, using an RNA or protein detection assay described herein. Additional clinical indications of successful treatment of myotonic dystrophy include improvements in muscle function, such as in the cranial, distal limb, and diaphragm muscles.

As used herein, the term “vector” refers to a nucleic acid, e.g., DNA or RNA, that may function as a vehicle for the delivery of a gene of interest into a cell (e.g., a mammalian cell, such as a human cell), tissue, organ, or organism, such as a patient undergoing treatment for a disease or condition described herein, for purposes of expressing an encoded transgene. Exemplary vectors useful in conjunction with the compositions and methods described herein are plasmids, DNA vectors, RNA vectors, virions, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

Myotonic dystrophy (DM) (e.g., DM), is a multisystemic disease, as described above. This example describes development of reagents and methods to treat, prevent, and/or ameliorate DM by simultaneously promoting MBNL1 expression and reducing (via e.g., via RNAi) DMPK repeat mRNA. It is demonstrated that the simultaneous action of both elements synergistically permit an effect using levels of each construct that are lower than what would be needed for each individually to achieve similar results. Accordingly, this combined gene therapy increases the safety of the therapeutic window with this combined gene therapy to regulate MBNL1 levels in treated tissues. Furthermore, the specific de-targeting of MBNL1 in cardiac muscle provides additional protection against damage to the heart. While this Example describes delivery systems focused on attenuation of the muscle system effects, the described approach can be applied to controlled delivery and expression in other tissues affected by the disease.

Approaches to Deliver a Combination Protein and RNAi Therapy with Regulation of

### MBNL1 Expression

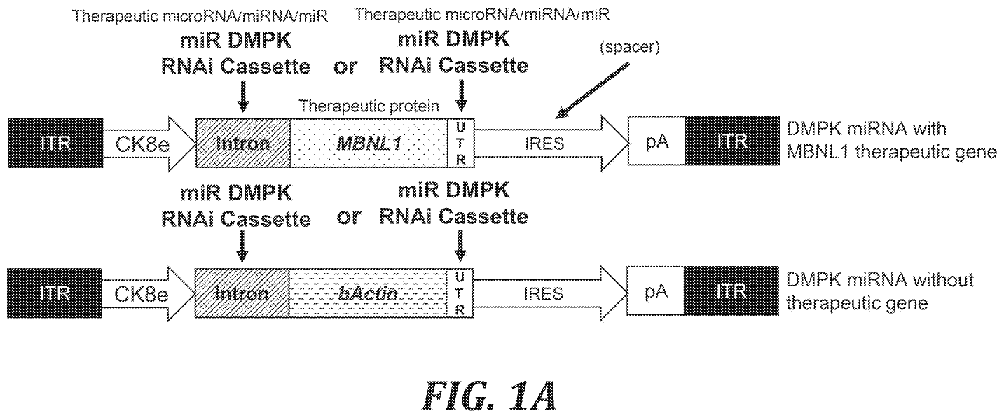

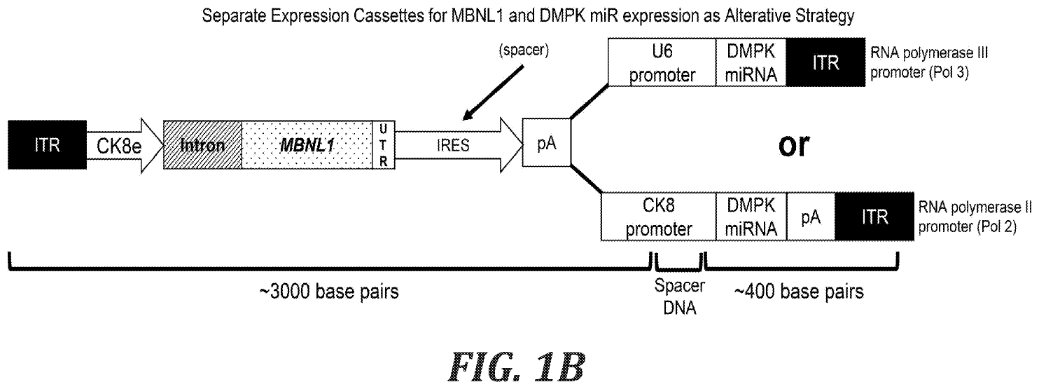

A unique aspect and advantage of this approach stems from the discovery that a combination therapy would require less expression of both heterologously encoded MBNL1 and anti-DMPK miRNA for efficacy, with less risk of toxicity for both the protein expression and RNAi expression cassette producing siRNA for DMPK mRNA reduction (and potential release of endogenous MBNL1). FIGS. 1A-1B schematically illustrate an exemplary gene delivery cassette for co-expression of MBNL1 in skeletal muscle and modalities to control expression of MBNL1. The inventors previously developed an AAV delivery vehicle for RNAi microRNA-based expression cassettes targeting the DMPK described in U.S. Patent Publication No. 20210269825A1, incorporated herein by reference in its entirety.

In this disclosure, such an exemplary vector would add, within the same vector (or optionally in a separate vector), either a separate or single muscle restricted RNA polymerase II promoter (Pol 2) expressing MBNL1 40 kd protein. If the DMPK RNAi expression cassette were separate from the MBNL1 expression cassette the DMPK RNAi expression cassette could be expressed from either 1) a strong RNA polymerase 3 promoter (Pol 3) or 2) the above-mentioned RNA Pol 2 promoters. Any of the muscle specific expression cassette (MSEC) promoters could be applicable and could modulate expression in cardiac and skeletal muscle at similar or different levels. For proof of concept, the inventors are using Creatine Kinase 8 (CK8) as the promoter of choice, which is in use in clinical trials for expressing microdystrophin.

### Gene Expression Cassettes:

Pol 3: Human snRNA U6 (small nuclear RNA [non-coding, involved in splicing in context of U6 small nuclear ribonuclear protein particle]) promoter driving expression of mir30a-based artificial gene for RNA interference (RNAi) that targets disease DMPK mRNA in myotonic dystrophy type 1 (DM1; could be used to express an RNAi RNA hairpin to target DM2 gene, CNBP or ZNF9). Exemplary interfering RNA are disclosed in U.S. Patent Publication No. 20210269825A1, incorporated herein by reference in its entirety.

Pol 2: several Pol 2 promoters including human β-Actin, Creatine kinase 8 (CK8), have been tested and muscle tropic promoters (Muscle Specific Expression Cassettes or MSECs) as discussed above, may also be utilized.

Typically, Pol 2 cassettes show 5-10-fold lower expression levels than Pol 3 promoters and factors controlling vector delivery will dictate whether the strength of these promoters is sufficient for efficacy in muscle with systemic delivery. Exemplary cassettes also include the MyoAAVs, which are 10-50× better at transducing muscle tissue with lower doses.

FIGS. 1A-1B, depict several approaches described above to deliver both the RNAi expression cassette and the MBNL1 gene cDNA (isoform 40; KIAA0428). Preferably, the RNAi encoding sequences are expressed in the context of the MBNL1 cDNA transcript with a single Pol 2 promoter driving expression of a contiguous mRNA containing MBNL1 and therapeutic pre-miRNAs to target the DMPK mRNA (or CNBP for DM2) (FIGS. 1A-1B). FIG. 1A also depicts therapeutic RNAi expression in the context of the skeletal muscle β-actin as a carrier gene that would not itself contribute any therapeutic benefit but would act as a carrier Pol 2 gene. FIG. 1B describes other exemplary arrangements of expression cassettes for vector gene delivery with separate promoters to express the repeat expanded mRNA (DMPK or CNBP).

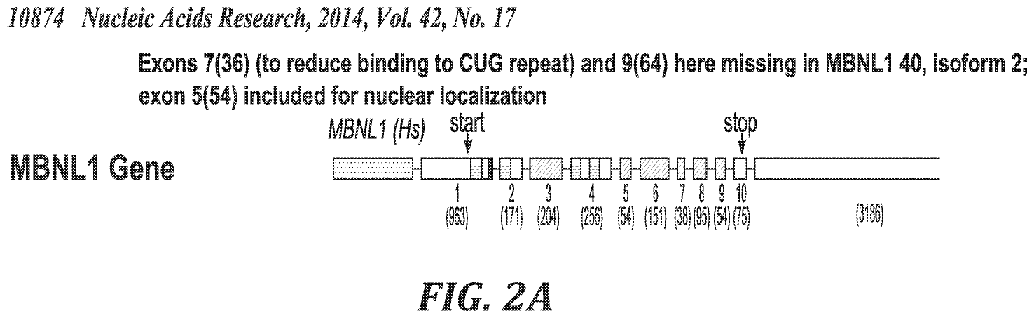

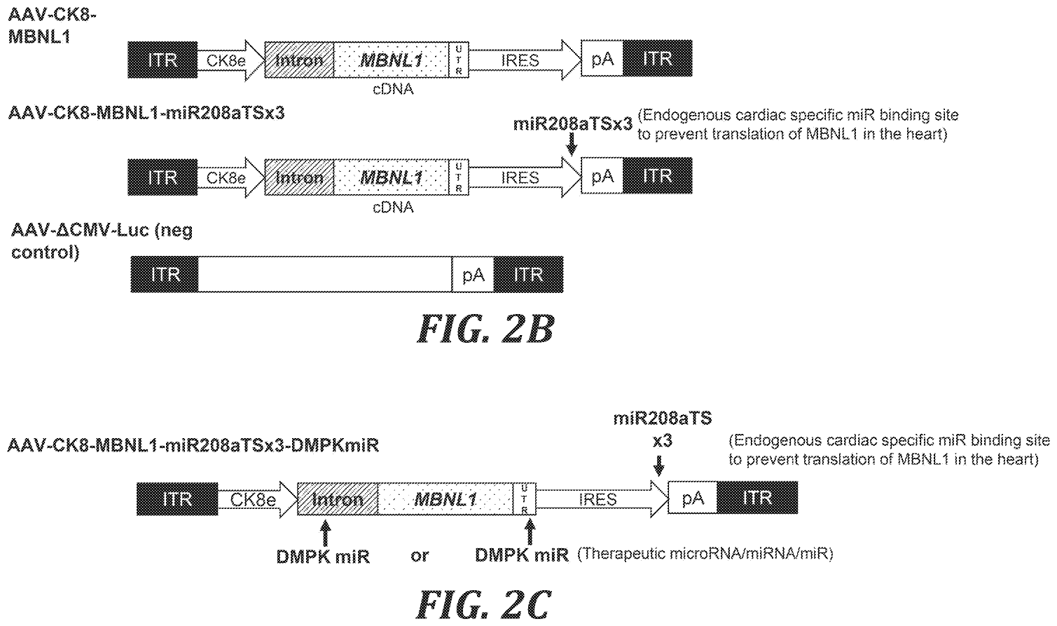

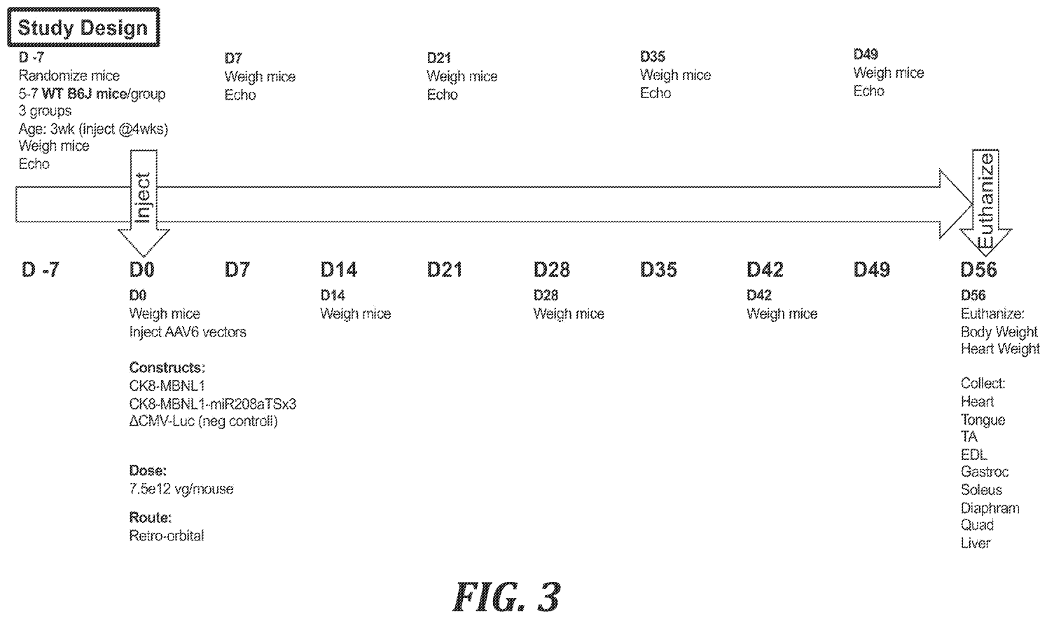

FIGS. 2A-2C illustrate methods to control MBNL1 gene expression levels in skeletal muscle and heart in steps to combine MBNL1 expression and DMPK RNAi as a systemic therapy. Schematic representation of the MBNL1 gene with 5′ UTR, exons, and 3′UTR depicted as boxes with exons and their lengths in DNA base pairs in parentheses indicated below (FIG. 2A). Translation start and stop codon sequences are labeled at top. The cDNA used for MBNL1 expression, MBNL1 40 (isoform 40) is a prevalent isoform and does not include exons 7 and 9 as indicated. The absence of exon 7 reduces toxic DMPK mRNA expanded repeat binding and inclusion of exon 5 is known to direct nuclear localization. For MBNL1 expression restriction to skeletal muscle, an AAV vector expression cassette containing the target sequence of the cardiac, endogenously expressed microRNA208a (miR208a) was engineered (FIG. 2B-2C). FIG. 2B also shows the negative control expression vector. This is the first approach to explore delivery of a MBNL1 expression cassette to provide levels of MBNL1 that do not cause toxicity in skeletal or cardiac muscle. Advantageously, the compositions disclosed herein can increase MBNL1 expression in vivo without any associated cardiotoxicity.

### Gene Expression Cassettes Modified to Control MBNL1 Expression:

Differential splicing between tissues contributes an additional layer of tissue specific regulation of protein expression. MBNL1 functions as an alternative splicing regulator in the nucleus and plays a role in mRNA localization in the cytoplasm that contributes to efficiency of translation of some mRNAs. MBNL proteins bind the sequence YGCY motifs (Y indicates pyrimidines) with their four paired CCCH zinc fingers, at sites in the pre-mRNAs they interact with to direct splicing, as well as the CUG (DM1) and CCUG (DM2) repeats at CG Watson-Crick base pairs of double stranded regions of the repeat expanded mRNAs. Levels of MBNL1 influence the inclusion of exon 5 of its own mRNA which was shown to influence the protein's cellular localization with a nuclear localization sequence encoded by this exon. Exon 5 is excluded at the highest rate in skeletal muscle (more than 2× amount in liver), but also in heart and thymus. This alternative splicing pattern of MBNL1 pre-mRNA itself suggests a higher demand of MBNL1 in these adult tissues. Exon 7 of human MBNL1 aids in multimerization in a ring-like structure when bound to repeat expanded, double-stranded hairpin RNA structures in DM1 tissues and causes MBNL1 self-association in yeast 2 hybrid studies. In FIGS. 1A-1B and FIG. 2A (detailed schematic drawing) MBNL1 40 kd protein producing gene construct used in the gene expression cassettes herein does not contain exon 7 (to reduce/dampen) binding to the toxic DM1 RNA hairpins or exon 9 in the prevalent 40 kd isoform. The presence of exon 5 directs nuclear localization for splicing correction.

In addition, MBNL1 itself can affect its own protein levels. Several published reports describe MBNL1's influence on its own biogenesis, through a mechanism known as autoregulation of gene expression. When levels increase over an equilibrium necessary for proper tissue splicing regulation MBNL1 binds to its first coding exon of its pre-mRNA and causes repression of translation (FIG. 2B). Based on this observation the inclusion of a sequence for binding MBNL1 early in a MBNL1 gene intended to supplement levels of nuclear MBNL1 could serve to regulate MBNL1 protein levels to achieve a safe and efficacious therapy, regardless of the variable level of vector-delivered genes in each muscle nucleus.

As a means of achieving proper levels of MBNL1 protein in treated DM muscle cells, inclusion of sequences to control MBNL1 levels could contribute to achieving safe levels while restoring and balancing alternative splicing in DM1 tissues. To control MBNL1 expression in skeletal and cardiac muscle two approaches were taken as follows: 1) inclusion of an endogenous miRNA binding site for binding a microRNA (miRNA) that is only expressed in cardiac tissues to reduce gene expression at the level of translation (the miRNA binding site or ‘target site=TS’ used here is miRNA208a in triplicate, but could be different to tune for non-human primates [NHPs] and humans); and 2) inclusion of a chimeric intron from the β-globin and immunoglobulin genes and/or sequences in the MBNL1 cDNA that serve as a MBNL1 regulatory binding site to prevent mRNA splicing and translation (FIG. 2C; description of each feature).

The present disclosure provides a novel approach for controlling MBNL1 expression both in skeletal muscle (MBNL1 binding and autoregulation) and in heart (miR208a binding site with miR208a expressed only in cardiac muscle) by combining: de-targeting the MBNL1 mRNA for translation inhibition with a repeated miRNA binding site; and adding a chimeric intron from genes likely controlled by MBNL1 for autoregulation and increased expression. The miR208a levels in the DM1 heart are not altered so expression levels are expected to provide miR208a binding to the target site in the engineered gene cassette. Methods for use of this sequence to treat a different muscular dystrophy are described in U.S. Patent Publication No. 20160058890. Variation of the placement of the miRNA target site and their number, for example placement in the 3′UTR sequences of the MBNL1 cDNA or in the chimeric intron or 5′UTR, can be used to optimize the control of MBNL1 expression with these DNA sequence elements, with exemplary options depicted in FIG. 2C.

### Example 2

### AAV-Mediated Systemic Expression of MBNL1 in Wild Type Mice

FIGS. 3-8 illustrate the evaluation of the effects of expressing MBNL1 in striated muscles of wild-type mice delivered by a AAV vector.

The study design to evaluate AAV-mediated systemic expression of MBNL1 in wild type mice with and without cardiac restriction is illustrated in FIG. 3. This study was conducted to assess the timeline and evaluating the safety of expressing MBNL1 in mice following AAV6-mediated systemic delivery. Wild type male and female C57BL6J 3wk old mice were randomized into 3 groups of 5-7mice, weighed, and cardiac function analyzed by echocardiogram 1 week prior to treatment. At 4 weeks of age, each group was retro-orbitally infused with 7.5×10¹² vector genomes (vg) of one of the experimental AAV vectors: CK8-MBNL1, or CK8-MBNL1-miR208aTSx3, as well as ACMV-Luciferase (ACMV-Luc [negative control]; lacking promoter activity due to deletion of CMV promoter sequences) serving as a negative control vector. Mice were weighed weekly and cardiac function was analyzed by echocardiogram at 1-, 3-, 5-, and 7-weeks post infusion. Mice were euthanized 8 weeks post infusion and tissues were harvested for analysis.

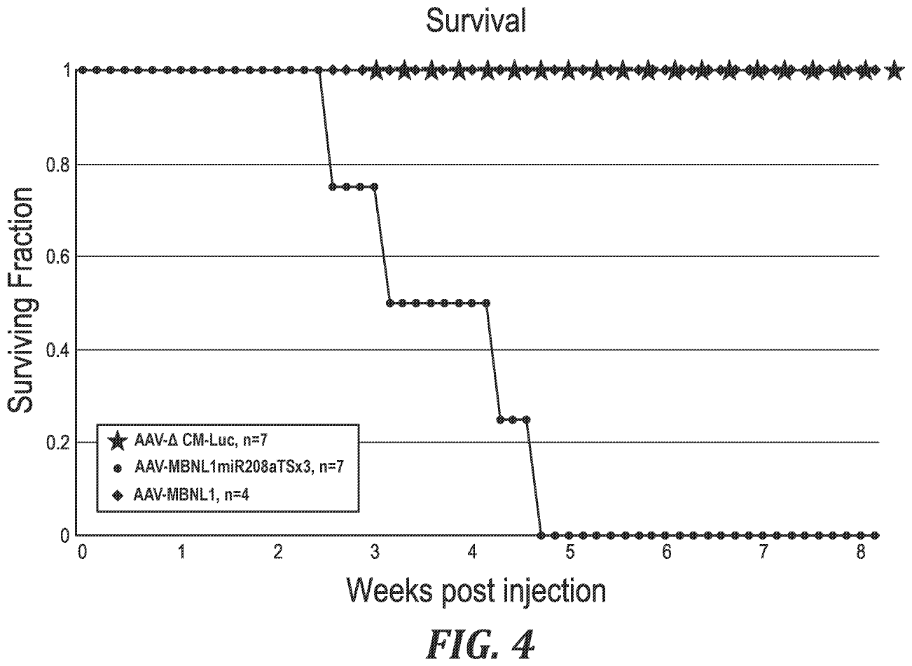

Mice receiving AAV-ACMV-Luc and AAV-MBNL1miR208aTSx3 appeared heathy and lived to meet the study endpoint at 8 weeks. In contrast, AAV-CK8-MBNL1 injected mice showed a rapid decline in body condition at about 2 weeks post infusion. Poorly functioning mice, as determined to have met humane end point criteria by research staff in consultation with facility veterinary staff after the first mouse died, were euthanized prior to the 8 weeks post injection study endpoint between 3-5 weeks post injection. Humane endpoint criteria included body condition scoring and echocardiogram evaluation. FIG. 4 illustrates survival assessment post treatment with MBNL1 and control vectors.

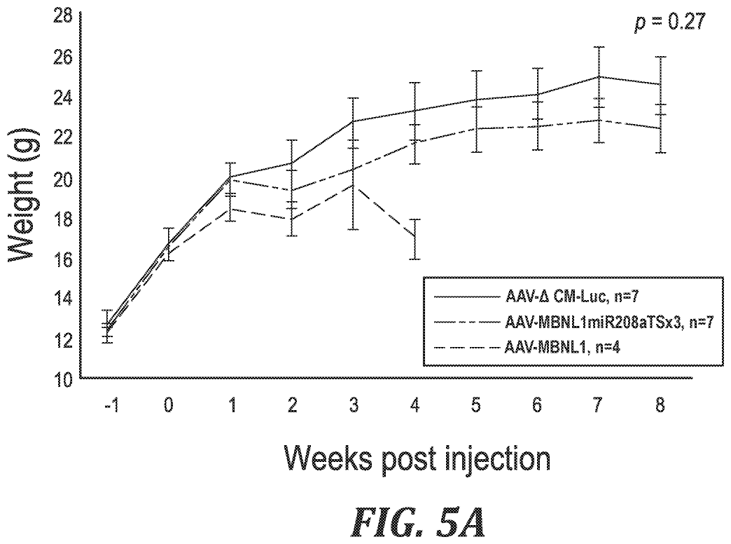

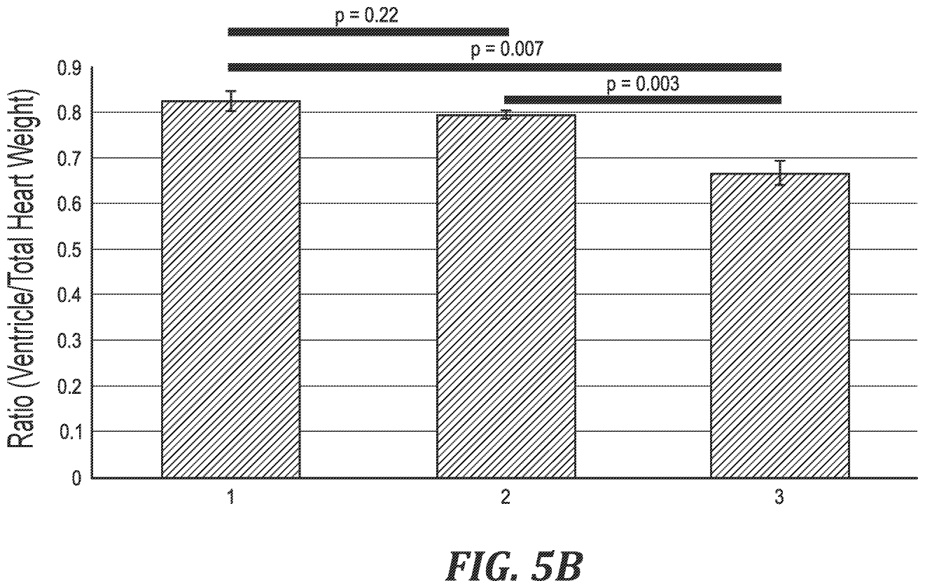

Mice receiving AAV-ACMV-Luc and AAV-MBNL1miR208aTSx3 appeared heathy and lived to meet the study endpoint at 8 weeks. In contrast, AAV-CK8-MBNL1 injected mice showed a rapid decline in body condition at about 2 weeks post infusion. Poorly functioning mice, as determined to have met humane end point criteria by research staff in consultation with facility veterinary staff after the first mouse died, were euthanized prior to the 8 weeks post injection study endpoint between 3-5 weeks post injection. Humane endpoint criteria included body condition scoring and echocardiogram evaluation. FIGS. 5A-5B illustrate body and heart weight monitoring with AAV-mediated systemic expression of MBNL1 in wild type mice with and without cardiac restriction.

The foregoing data and results validated the combinatorial approach design, i.e., combining RNAi and controlled MBNL1 expression, using the AAV-delivered MBNL engineered gene cassettes shown in FIGS. 2A-2C.

### Example 3

To test the expression of MBNL1 in vivo C57BL/6 mice were injected by the retro-orbital route with AAV6-MBNL1/intron/miR208a binding site (AAV-MBNL1-miR208aTSx3) at a dose of 7.5×10¹² vector genomes (vg) at 4 weeks of age. These mice were compared to AAV-ACMV-Luciferase (non-expressing negative control vector) and AAV-MBNL1 (no miR208aTS) and were monitored by weight and echocardiography longitudinally over 8 weeks as outlined in FIG. 3 as ‘Study Design.’ Without the miR208aTS sequences mice began to die after 3 weeks of age, with the whole cohort with unrestricted MBNL1 expression (no miR208a heart specific target site) died by 5 weeks post injection as shown on the Kaplan-Meier curve to assess mortality (FIG. 4). Death was attributable to gene delivery of MBNL1 (uncontrolled expression), since the no-expression negative control vector AAV-1CMV-Luc did not appear toxic. Importantly, MBNL1 expression with the miR208a target site (AAV-MBNL1miR208aTSx3) to restrict heart expression did not affect the lifespan of the treated mice over the course of the study. Body weight plateaued at 1 week after injection in the AAV-MBNL1 mice before they began dying, while the AAV-MBNL1-miR208aTSx3 and AAV-ACMV-Luciferase mice continued to gain weight and did not show a significant difference in weight at the end of the study (FIG. 5A).

Similarly, there was a reduction in ventricle to total heart weight for MBNL1 expression, whereas restriction of MBNL1 in cardiac tissue did not significantly reduce the ventricle to total heart weight (FIG. 5B). In overall health monitoring it was clear that the miR208aTSx3 sequence had a significant impact in preventing the toxic effects of MBNL1 expression with survival and weight measurements comparable to control treated mice.

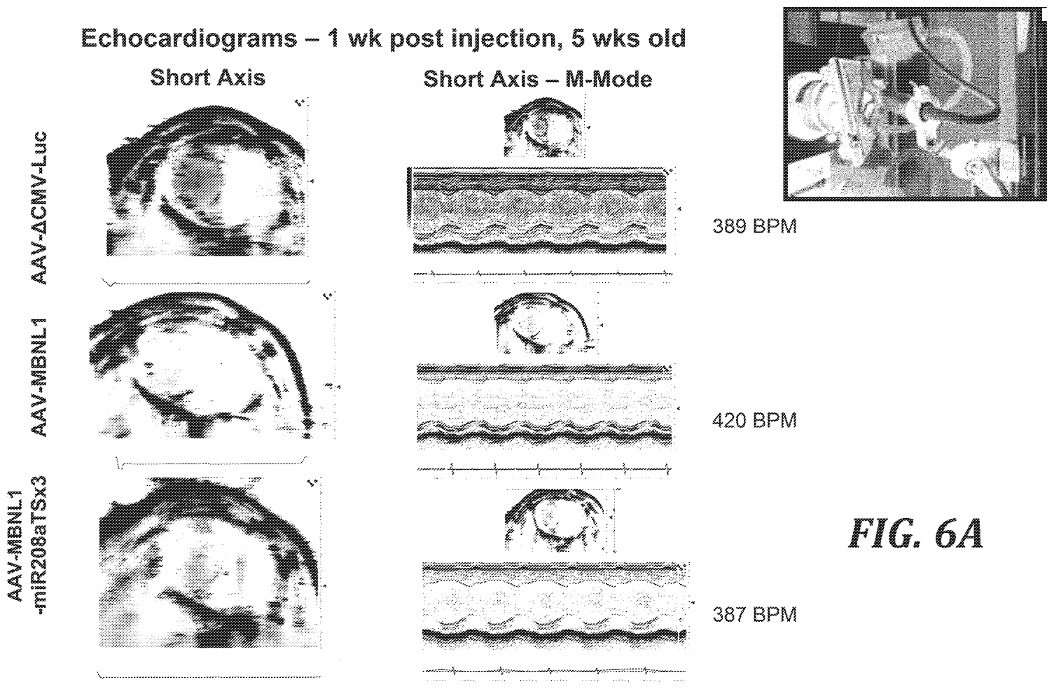

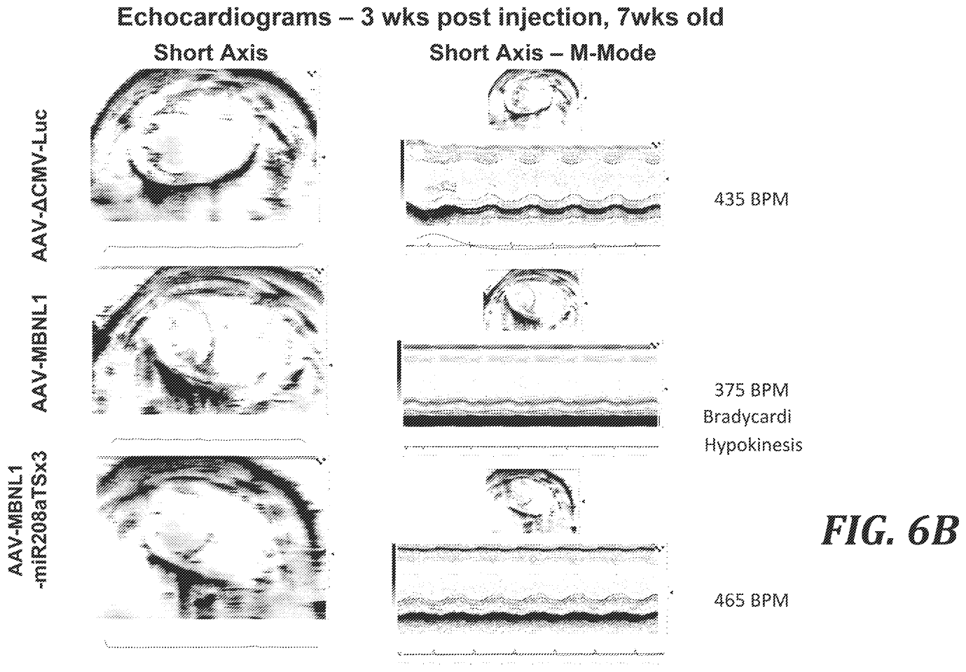

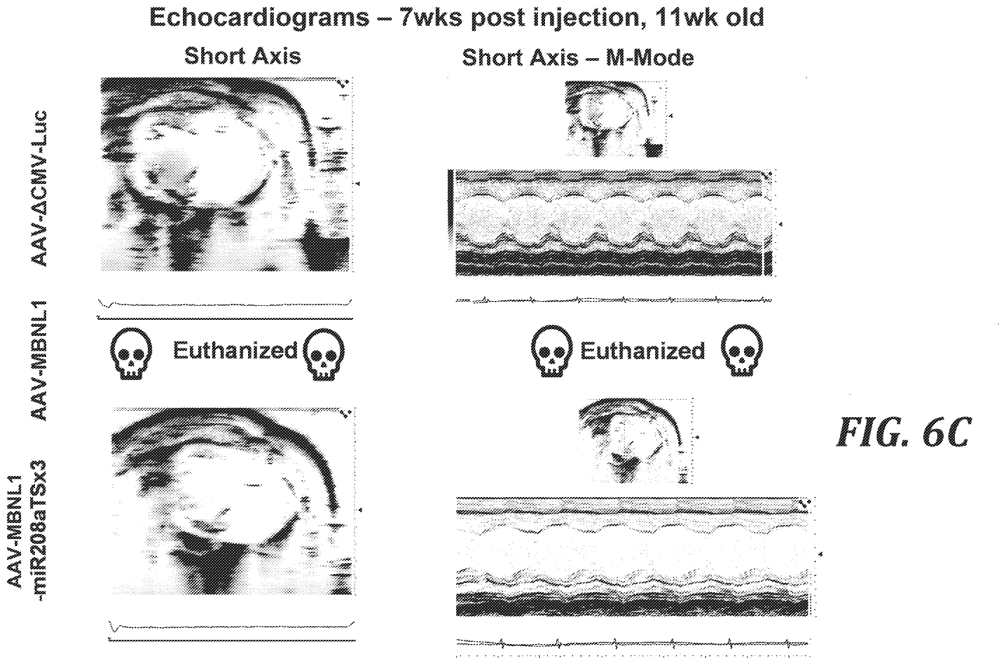

FIGS. 6A-6C illustrate echocardiographic examination of AAV-mediated expression of MBNL1 with and without cardiac restriction. During the time course of the experiment mice were also monitored by echocardiography at 1, 3, and 7 weeks (FIGS. 6A, 6B, and 6C, respectively, post-treatment). Echocardiogram traces are similar in AAV-ACMV-Luciferase and AAV-MBNL1-miR208aTSx3 treated mice at 7 weeks, whereas none of the AAV-MBNL1 mice survived past 4.75 weeks. In contrast, the MBNL1 injected mice showed severe bradycardia (slow heart rate) and hypokinesis (reduced range of movement) at 3 weeks as they were succumbing to the cardiotoxicity. There may be some minor changes in the AAV-MBNL1miR208aTSx3 mouse heart, such as a slightly slower heartbeat (but w/in normal 10% fluctuations), which could result from higher amounts of AAV6 transducing cardiac tissue vs. skeletal muscle, which is typically 5× higher compared to skeletal muscle. Lower doses of the vector are contemplated for therapeutic use for efficacy with the combined expression of MBNL1 and DMPK mRNA-targeted RNAi, combined with more effective vectors (i.e. engineered AAV capsids provide greater transduction with less vector) to avoid any potential deleterious effects in cardiac tissue.

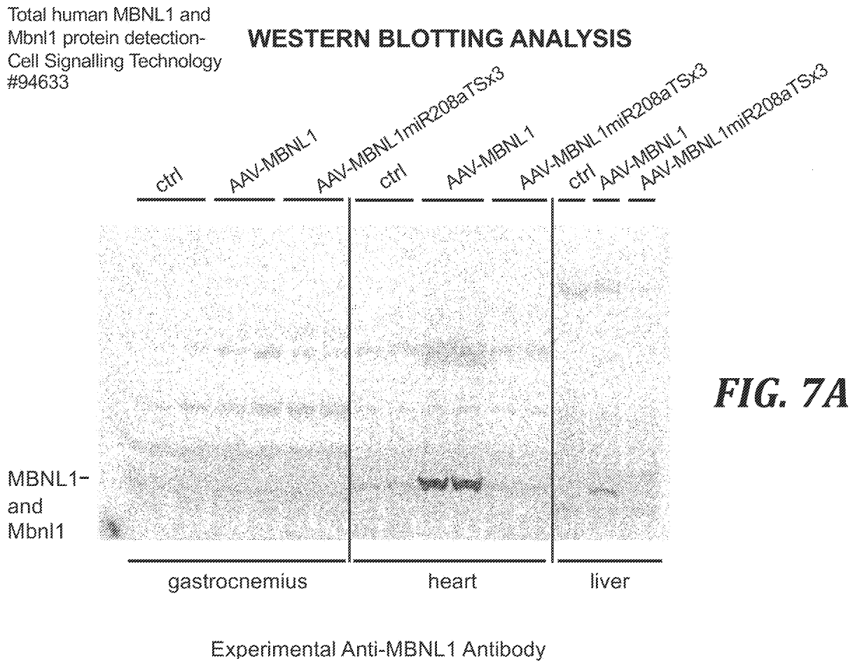

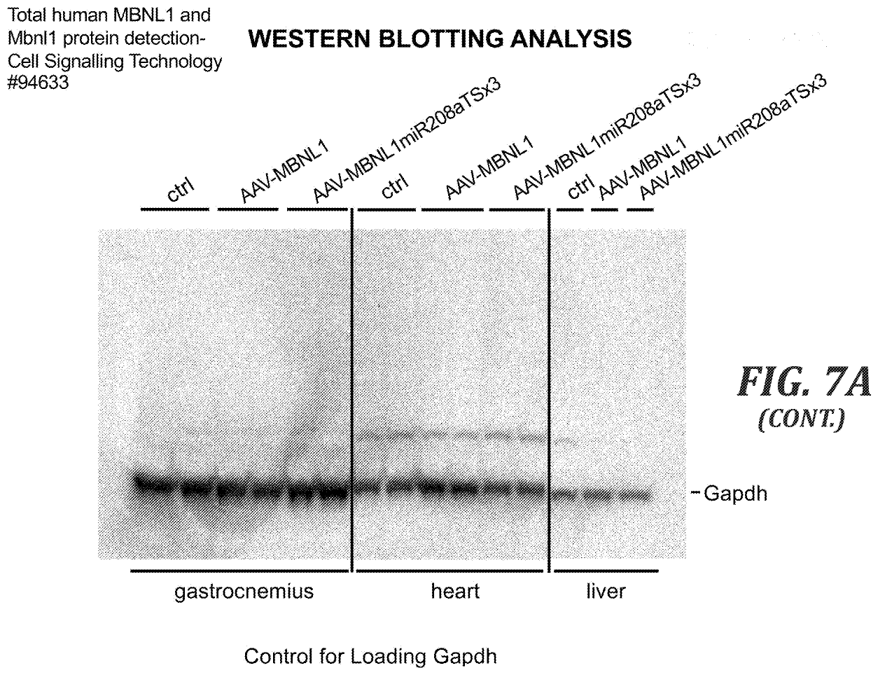

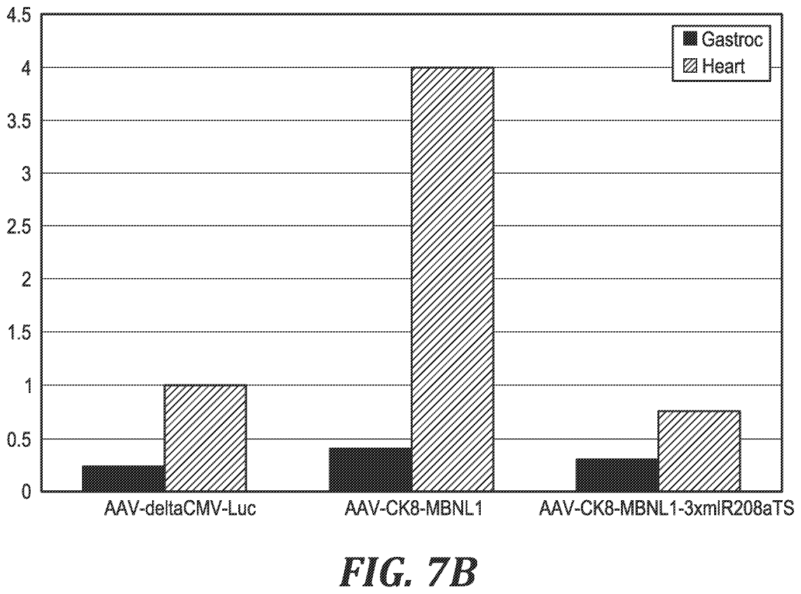

FIGS. 7A-7B illustrate immunoblot quantitation of MBNL1 protein expression levels of MBNL1 and control vectors at study endpoint. These additional assays were conducted to assess level of MBNL1 expression in skeletal, cardiac, and liver tissues following systemic injections of control, MBNL1, and MBNL1-miR208aTSx3 vectors. Protein lysates were made from gastrocnemius muscle, heart and livers of injected mice and analyzed by immunoblotting. Two different antibodies, the commonly used A2764 anti-MBNL1 polyclonal antibody (gift of C. Thornton, U Rochester, NY) and anti-MBNL1 polyclonal antibody #94633 (Cell Signaling Technology, Danvers MA) which resulted in similar patterns of MBNL1 detection on Western blot analysis of the tissue lysates (FIG. 7A). These antibodies detect both mouse endogenous and AAV-delivered human MBNL1 protein. Quantitation of total mouse and Mbnl1 and human MBNL1 expression in heart from lysates of AAV-MBNL1 systemic administration showed over a 4-fold increase in MBNL1 protein in heart compared to the control vector, whereas the AAV-MBNL1mir208aTSx3 injected hearts were 80% of the control (FIG. 7B).

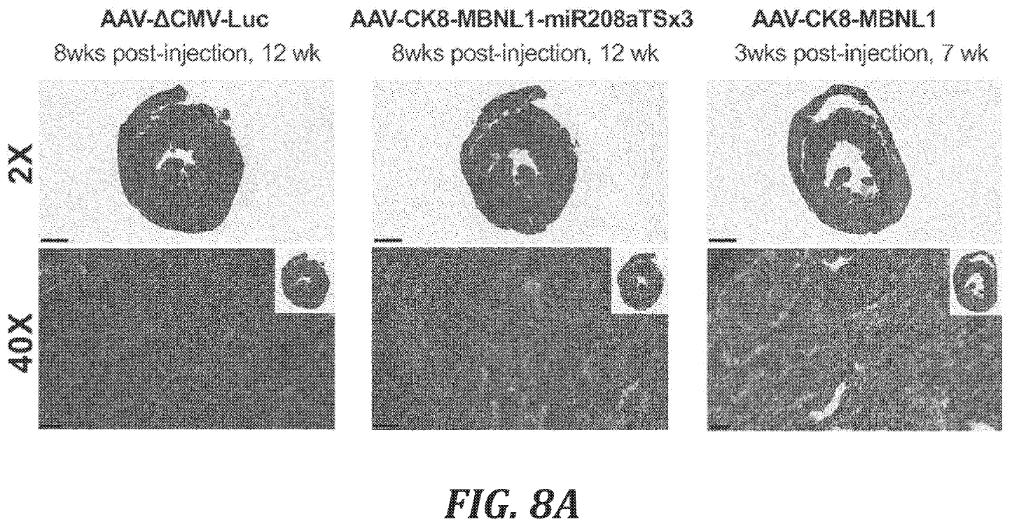

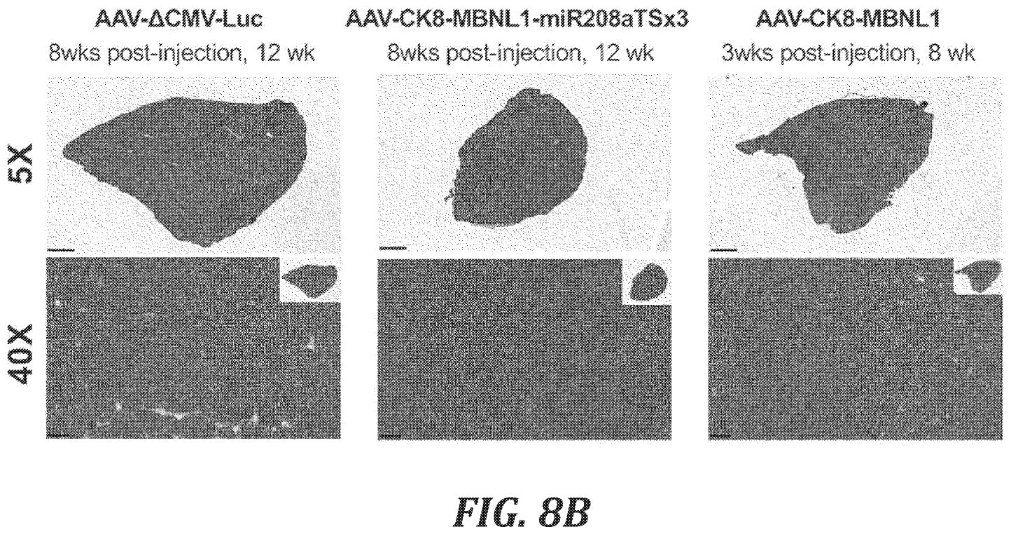

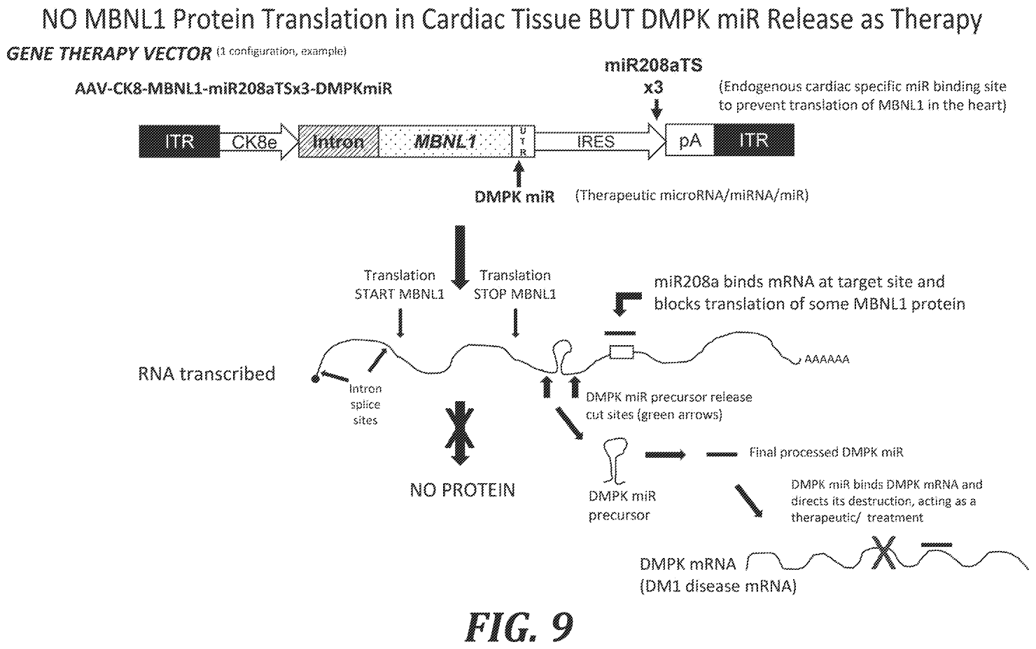

FIGS. 8A-8B illustrate striated muscle tissue structural analysis for assessment of AAV-mediated MBNL1 protein expression effects. To evaluate the tissue effects of MBNL1 expression striated muscle transverse cryosections were stained with hematoxylin and eosin (H&E) to look for potential structural changes soon after euthanasia of the mice and cryopreservation of tissues.

Strikingly, severe dilation of the ventricle and was apparent in the mice that were injected with AAV-MBNL1 compared with the AAV-ACMV-Luc control and AAV-MBNL1miR208aTSx3 (FIG. 8A). Accompanying the dilation in the AAV-MBNL1 treated mice were cardiac fiber changes indicating damage, marked by lighter hematoxylin staining regions with gaps between fibers displaying a disorganized appearance. Not surprisingly the AAV-MBNL1 mice became lethargic and showed deficits on echocardiography and were euthanized at a humane endpoint as determined by institutional IACUC standards at 3-5 weeks post injection. No skeletal muscle changes in fiber size or centralized nuclei that would indicate damage and repair had occurred, were observed. Rather, skeletal muscle fibers appeared normal in size and nuclei appeared at the fiber periphery, typical of unaffected muscle, for all of the injected mice.

These results indicate that MBNL1 overexpression in naïve wild type hearts leads to cardiopathology associated with cardiac insufficiency and death and establish the need for the controlled expression of MBNL1, as demonstrated herein, as an approach for using MBNL1 as a therapeutic either alone or in a combination with other therapies. The inventors demonstrate a safe and effective therapy that can prevent cardiac overexpression of MBNL1 using a cardiac-specific microRNA (miR) target site, such that expression of the corresponding miR only in cardiac tissue prevents expression (gene silencing) through the RNA interference pathway that functions to control gene expression, by preventing translation of mRNAs with homologous sequences in the cardiac tissue.

### Example 4

Skeletal Muscle-Restricted MBNL1 Expression in the Human Skeletal Actin Long Repeat Mouse Model (HSA^{LR}) of DM1 to Ameliorate Alternative Splicing Caused by the DMPK mRNA Repeat Expansion

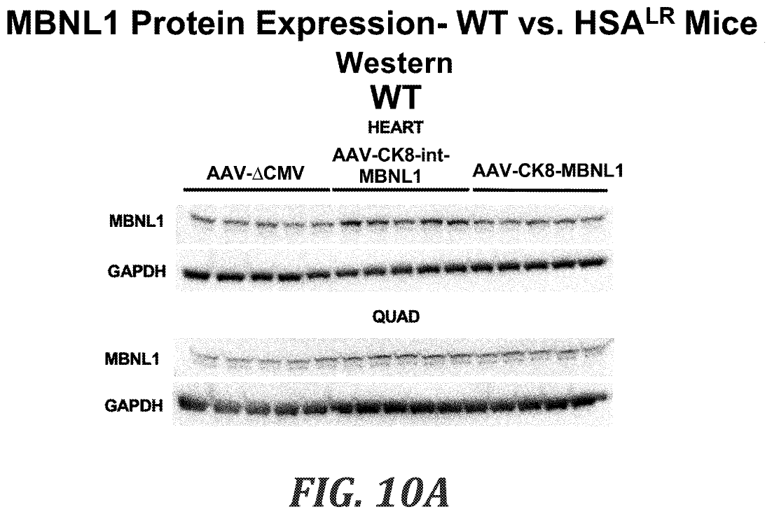

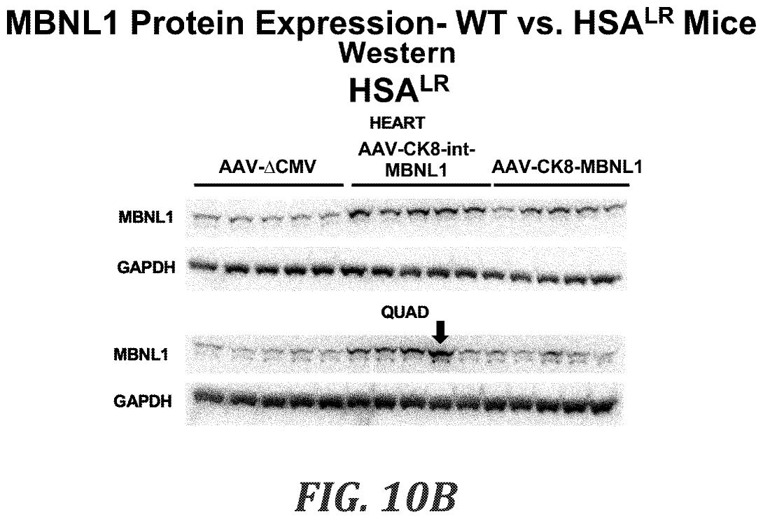

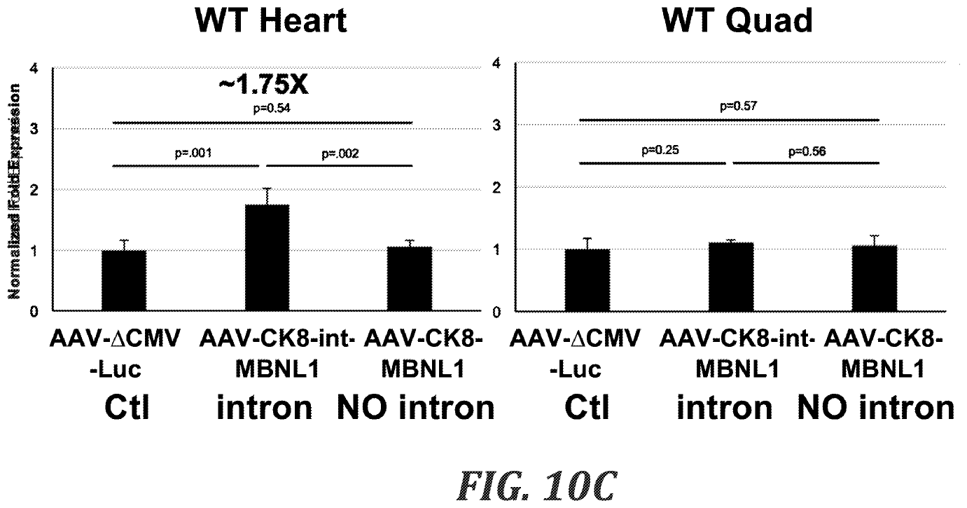

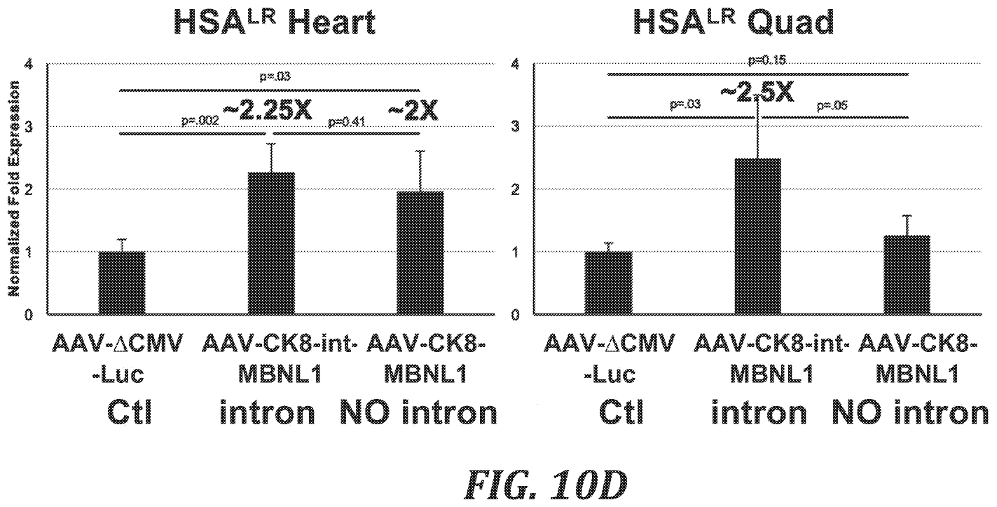

The combination of MBNL1 expression and miR208 target site binding was tested for safety and efficacy in HSA^{LR} mice. Quantitation of the total level of MBNL1 (antibody binds both mouse and human proteins) was accomplished by western blot analysis of wild-type (FIG. 10A) and HSA^{LR} (FIG. 10B) mice injected with AAV6-MBNL1 vectors either containing or lacking an intron sequence to evaluate the effect of the intron on expression levels in the heart and quadriceps muscle. FIGS. 10C and 10D represent bar graphs of quantitation for wild-type and HSA^{LR} mice for expression of MBNL1 in heart (left panel) and quadricep muscle (quad) (right panel), respectively.

Vector treated wild-type mice showed a slight increase in MBNL1 in heart tissues and no upregulation of MBNL1 in the quadriceps (quad) muscle (FIG. 10C, bar graph of quantitation shown). Vector (and delivered gene) levels are typically 10-fold higher in cardiac muscle with AAV6; although an expected reduction of levels to 1.75-fold and 2.5-fold higher (compared with 10.5-fold without the miR208aTSx3 site in FIGS. 7A-7B) were observed with the presence of the mir208aTSx3 binding element included in both vectors in heart tissue of wild-type (FIGS. 10A and 10C) and HSA^{LR} mice (FIGS. 10B and 10D).

The lack of MBNL1 overexpression in the quadriceps muscle of wild-type mice potentially is a response of the MBNL1 gene to the maximal level of functional MBNL1 in normal skeletal muscle, even with the strong CK8 promoter expected to produce high levels of protein. When endogenous MBNL1 is at functional levels, as it is in normal mice, then the MBNL1 gene is repressed. In the HSA^{LR} mouse, however, a proportion of functional MBNL1 is bound by the repeat-expanded HSA mRNA (HSA is platform gene with expanded repeat that leads to skeletal muscle disease phenocopy) causing a functional deficit of MBNL1 protein. The level of MBNL1 is elevated 1.0-4-fold (FIG. 10B, arrow indicates highest expression) in the treated HSA^{LR} quadriceps muscles from the cohort of mice treated (mean=2.5), where functional MBNL1 is lacking. The combined observations of the lack of upregulation of MBNL1 in wild-type mice and the increase in the HSA^{LR} mice suggest that the MBNL1 transgene is autoregulated to approximate homeostasis in both mouse lines.

An AAV-delivered muscle MBNL1 expression cassette (Creatine kinase promoter version 8, CK8) with miR208a target sites, to prevent elevated expression in heart, in the HSA^{LR} mouse model was tested. HSA^{LR} model is based on skeletal muscle expression of a human skeletal actin gene with the same DNA repeat sequences found in the DMPK mRNA in DM1 in the same mRNA location in the 3′ untranslated region. This model does not express the repeat-containing mRNA in the heart; therefore, only the ability of the vector to mitigate alternative splicing defects in skeletal muscle in HSA^{LR} mice was tested. As in wild-type mice systemic delivery could cause toxicity in cardiac tissue with overexpression of MBNL1, so the miR208a target binding sites were included to prevent cardiac dysfunction. In the previous vector tests upregulation of MBNL1 protein expression in skeletal muscle of wild-type mice was not observed.

In this study, a vector expressing a MBNL1 protein that was deleted for the chimeric β-globin/immunoglobulin gene intron, was included to circumvent potential repression being mediated by MBNL1 binding to the intronic DNA or RNA sequence. It was speculated that MBNL1 could bind to this intronic sequence to block upregulation of MBNL1 protein production in an autoregulatory manner. MBNL1 is known to regulate immunoglobulin genes. Alternatively, sequences in the intron could interfere with endogenous MBNL1 cDNA sequences included for autoregulation of MBNL1 expression for control of therapeutic functional MBNL1 protein levels. In the current experiment vectors with either CK8-intron-MBNL1 miR208a target sites (CK8-intron-MBNL1) (i.e., MBNL1 containing the chimeric β-globin/immunoglobulin gene intron), CK8-MBNL1 miR208a target sites (CK8-MBNL1; MBNL1 without the chimeric β-globin/immunoglobulin gene intron), and a control promoter less luciferase vector (ACMV-Luc) were tested.

The results show that inclusion of the chimeric β-globin/immunoglobulin gene intron did indeed increase MBNL1/Mbnl1 total expression (no specific antibody to human MBNL1 is known to exist), rather than inhibiting overexpression (which would increase MBNL1/Mbnl1 levels above the gene cassette including the intron) with MBNL1 binding and feedback loop suppression (FIGS. 10A-10D). FIG. 10B shows western analyses of heart lysates (top panel), and quadriceps muscle (bottom panel) for immunodetection of MBNL1 protein. The bar graph representing the increase in MBNL1/Mbnl1 expression in each tissue is also shown (FIG. 10D).

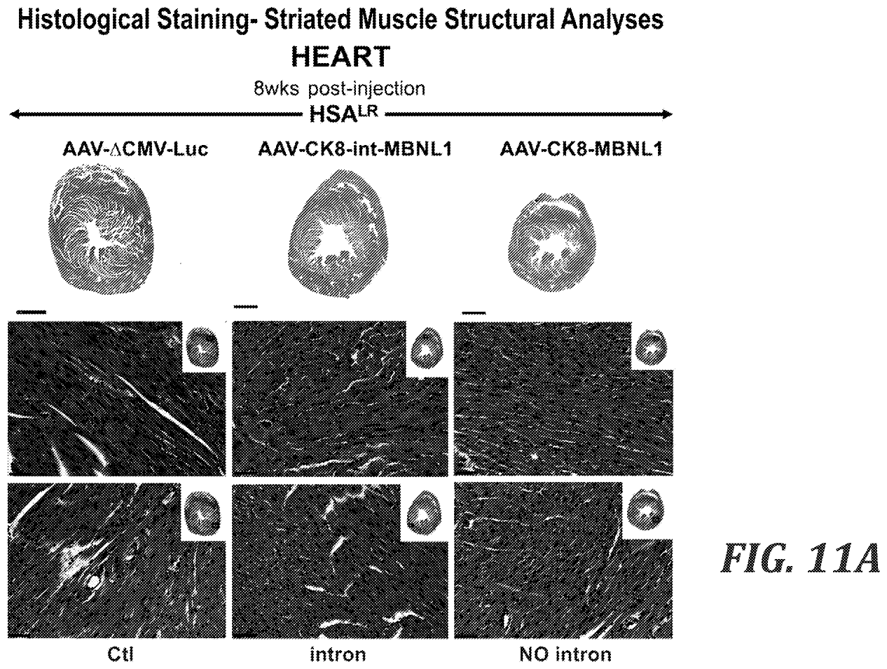

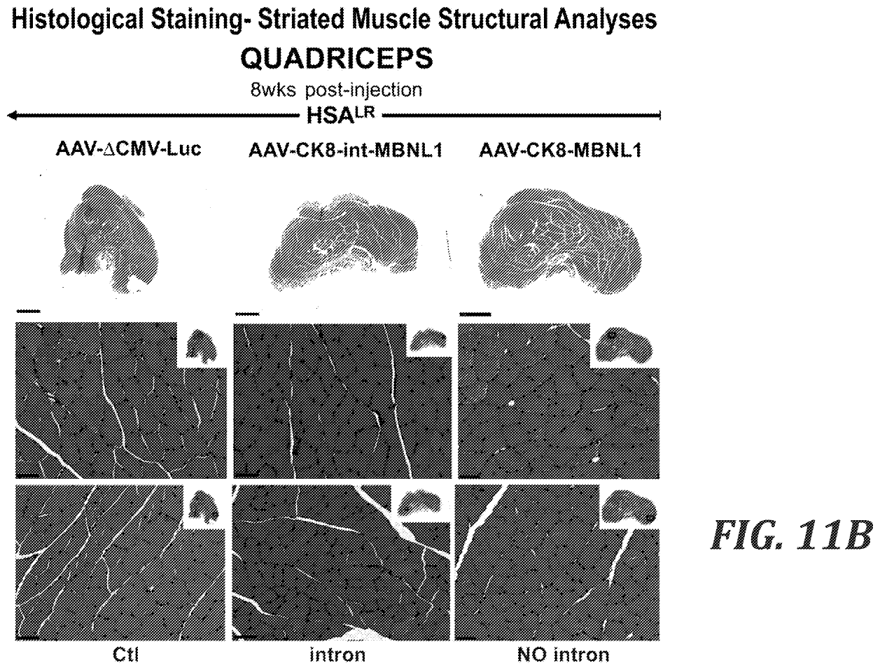

The contribution of MBNL1 with delivery of the CK8-MBNL1-intron vector to the total MBNL1/Mbnl1 ranged from 1.75 to 2.25-fold higher than the control ACMV-Luc vector in the hearts of wild-type and HSA^{LR} mice (n=5 each group) appears on histological staining with H&E to not be enough to cause toxicity (FIG. 11A, left panel). The inclusion of miR208a binding sites prevent high level expression in heart as seen previously using vectors without the miR208a sites in wild-type mice (FIG. 10A). Further, no upregulation of MBNL1 in the wild-type mouse quadriceps, was observed, supporting the hypothesis that MBNL1 and/or Mbnl1 are autoregulating the transgene and preventing expression when sufficient MBNL1/Mbnl1 is present.

Conversely MBNL1/Mbnl1 total levels are increased in the HSA^{LR} mouse quadriceps, presumably because of the reduction in free MBNL1/Mbnl1 needed to achieve equilibrium for efficient splicing in the cell. The quadriceps muscle appeared normal on histological examination (FIG. 11B, right panel). The MBNL1 expressed from the vector appears to have supplemented the level of free MBNL1 until enough free and functional total amount of MBNL1/Mbnl1 (-sequestered Mbnl1 by the repeat expanded DMPK mRNA) was achieved resulting in overall totals exceeding the baseline HSA^{LR} Mbnl1 levels (+sequestered Mbnl1). The five CK8-intron-MBNL1 vector-treated mouse quadriceps showed upregulation of MBNL1/Mbnl1 ranging from 1.5-6-fold, which will be considered in the context of the relationship of total amount of protein and its relative effect on alternative splicing as a readout of therapeutic efficacy.

### Example 5

### Therapeutic Efficacy

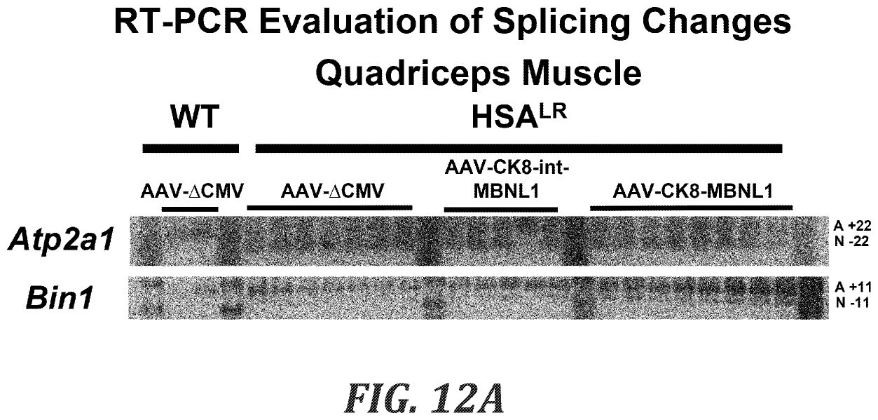

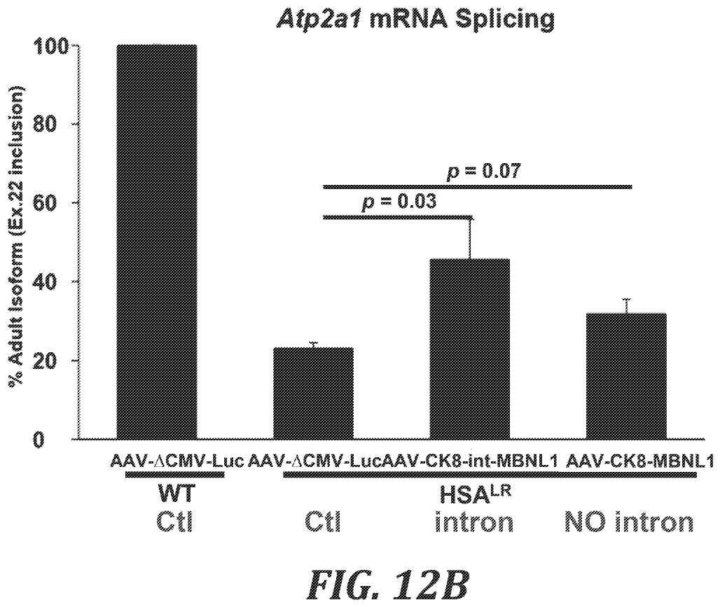

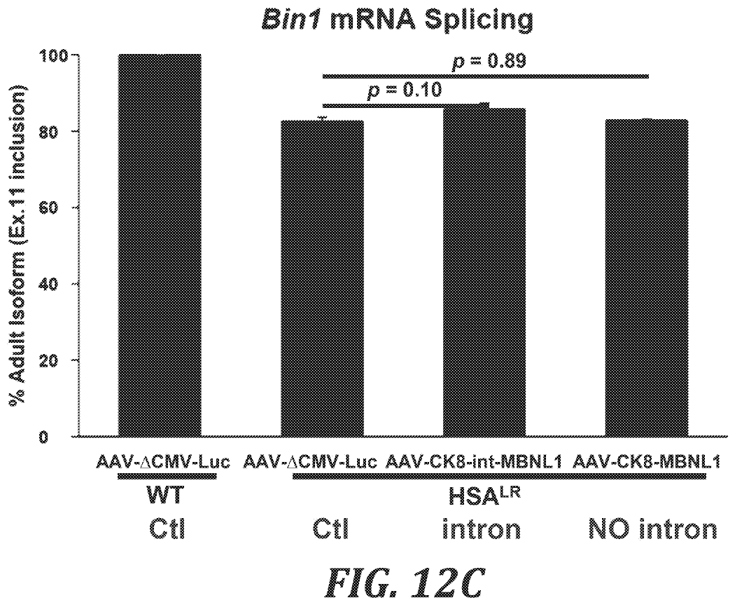

Two well-characterized alternatively spliced genes affected by the level of functional MBNL1/Mbnl1, Atp2a1 and Bin1, were examined (FIGS. 12A-12C). RT-PCR analyses of transcripts from quadriceps with low PCR cycle number (25) showed alternative splicing changes that were significant for both the vector-derived MBNL1 expression cassettes with or without the intron for Atp2a1 alternative splicing (FIGS. 12A-12B). The results indicated that both MBNL1 expression cassettes increased the level of splicing correction indicated by the increase presence of exon 22, likely due to the increase in MBNL1 protein expression, although the vector MBNL1 expression cassette with the intron did increase the correction of missplicing more (˜2-fold), reaching significance (p<0.05) compared to the vector without the intron. There appears to be a correlation with the increase in MBNL1/Mbnl1 protein and the highest level of correction for quadriceps sample 4 protein isolated from the cohort treated with the vector including the intron. Notably, the quadriceps is a muscle that is difficult to treat with systemic AAV6 gene delivery. A minimal shift in alternative splicing was noted for Bin but did not reach a level of significance for this assay (FIGS. 12A and 12C). These data support the hypothesis that MBNL1 supplementation at levels achievable with AAV-CK8-intron-MBNL1 systemic delivery can improve splicing outcomes in a mouse model of DM1, suggesting that this approach could be optimized to increase the therapeutic potential.

### Example 6

DMPK miRNA Activity Quantitation in HEK293 and DM1 Myogenic Precursor Cells

Two cell screening platforms were assessed for DMPK miR activity defined as reduction of the DMPK mRNA. Therapeutic targeting and reduction of the DMPK mRNA carrying the toxic repeat by RNA interference in myotonic dystrophy patient-derived cells reverses the pathological changes in these cells and in animal models. In the first example (FIG. 13A) HEK293 cells, which express low levels of DMPK mRNA, were transfected with DNA plasmids containing Pol 3 U6 DMPK miR expression cassettes in a small study to screen DMPK miRs designed and manufactured in the laboratory for reduction of DMPK mRNA. When compared to transfection of a plasmid lacking a miR several DMPK miR-containing plasmids were able to reduce the level of DMPK mRNA. In a second example of a cell-based screen DM1 myogenic precursors were infected with AAV6 vectors carrying some of the same and additional Pol 3 U6 DMPK miR expression cassettes. These DM1 myogenic precursors were made from patient fibroblasts converted to induced pluripotent stem cells and induced to form muscle-like cells that possess characteristics of DM1 muscle cell pathology, such as DMPK expanded repeat mRNA, nuclear foci, and splicing defects, and can be differentiated to form myogenic syncytial fibers in cell culture dishes (Mondragon-Gonzalez, R, and Perlingeiro, R. C. R. Recapitulating muscle disease phenotypes with myotonic dystrophy 1 induced pluripotent stem cells: a tool for disease modeling and drug discovery. Disease Models & Mechanisms (2018) 11, dmm034728. doi: 10.1242/dmm.034728). AAV6 DMPK miR vectors were applied to cells and then the cells were differentiated for 5 days and RNA was isolated for RNA sequencing. Analysis of the pA enriched RNA showed varying levels of DMPK mRNA reduction plotted as bar graphs in FIG. 13B. The DMPK miR vector with miR sequence 97 showed a 43% reduction of DMPK miR relative to the highest DMPK levels in the screen. Quantitation of DMPK mRNA was assessed relative to control mRNAs that are typically used as internal quantitation standards, β-actin, GAPDH, and RPS9. Vector 8 displayed knockdown activity in both cell assay platforms and vector 97 showed greater activity compared to vector 8 in the DM1 myogenic precursors. These techniques for cell screening of DMPK miR activity are employed to identify DMPK miR sequences for use in a combination therapy.

The inventors successfully demonstrate that combining RNAi to target the DMPK mRNA for destruction in combination with controlled expression/supplementation with MBNL1 in limited amounts could provide beneficial improvements in muscle and, with more widespread gene delivery, a wide variety of tissues affected by myotonic dystrophy.

Experimental assessment of controlled MBNL1 expression in muscle is critical for achieving an effective combination therapy for DM. The inventors show that muscle-restricted expression with an MSEC promoter, CK8, combined with an intron-based autoregulation of MBNL1 in skeletal muscle and reduction of expression in heart with a heart-specific miRNA binding site, lead to a safety profile compatible with MBNL1 supplementation as part of a MBNL1 protein expression/RNAi targeting therapy. Further, the data presented herein also shows that overexpression of MBNL1 in hearts of wild type mice lead to cardiotoxicity and death. This highlights the importance of employing strategies to restrict MBNL1 in the heart.

In addition, these studies have also revealed that MBNL1 is limited in skeletal muscle, likely due to inclusion of an intron that binds MBNL1 itself for autoregulation. Thus, exogenous autoregulated expression of MBNL1 in skeletal muscle will potentially provide additional nuclear supplemental MBNL1 protein (MBNL1 40 kd isoform is nuclear), although an increase in total human MBNL1 and mouse Mbnl1 was not observed in these studies.

MiRNA restriction of MBNL1 protein expression in cardiac tissue occurs at the level of translation inhibition, so therapeutic DMPK RNAi knockdown could potentially occur with DMPK RNAi processing from the MBNL1 transcript including the DMPK RNAi RNA hairpin sequences.

The present disclosure also demonstrates the ability of the AAV6-CK8-intron-MBNL1 vector applied as a systemic therapy to provide MBNL1 protein expression to the MBNL1/Mbnl1 total protein in the HSA^{LR} mouse model of DM1. With an expression increased ˜6-fold relative to untreated HSA^{LR} mice one can achieve full correction of Atp2a1 splicing to wild-type mouse levels. Potentially, levels between 6-10-fold will provide sufficient correction of many misspliced mRNAs due to the reduction of functional MBNL1. The muscle expressed MBNL1 isoform was selected because of studies that provided indications of activities suitable for therapeutic application. This MBNL1 protein produced from the vector was characterized as a version of the MBNL1 protein that lacks dimerization ability to prevent binding to the expanded repeat RNA, and can localize in the nucleus, and bind to its cognate transcript for potential negative autoregulation.

The data herein suggests MBNL1 autoregulation pathway is active and the transgene mRNA is responsive in the disease context, since an increase in total MBNL1/Mbnl1 in vector treated HSA^{LR} muscle and not wild-type muscle was observed. MBNL1 is produced in HSA^{LR} skeletal muscle to compensate for the lack of functional Mbnl 1 in the DM1 disease model, but not in wild-type mice where Mbnl 1 functional levels are in equilibrium with the levels needed for splicing and other functions. The present disclosure contemplates combining the MBNL1 expression cassette with DMPK RNAi sequences in newly developed AAV vectors for an approach that would ultimately 1) degrade the DMPK mRNA (RNAi); 2) provide additional functional MBNL1 to correct defective alternative splicing; 3) provide a safeguard to prevent MBNL1 expression in cardiac tissue; and 4) attain more uniform transduction of skeletal muscle with lower doses using emerging engineered AAV capsids for less variability in effect. The present disclosure contemplates utilizing new myotropic vectors to achieve improved efficacy with systemic delivery. An exemplary myotropic vector contemplated by the present disclosure includes MyoAAVs. The use of such vectors is potentially useful for lowering the ratio of cardiac to skeletal muscle transduction and reducing liver transduction due to lower doses necessary to transduce muscle.

Efficacy from controlled nuclear MBNL1 expression, along with degradation of DMPK mRNA by RNAi to reduce the level of toxic CUG repeat expanded DMPK mRNA, is expected to be a more potent approach than either method alone. Dose-limiting toxicity with more potent vectors are expected to provide a dose range that will alleviate safety concerns to achieve the ultimate DM treatment goal, which is the increase in splicing and other activities of MBNL1 and effects of accumulation of toxic DMPK mRNA with expanded CUG repeats (or CCUG for CNBP RNA, DM2) to return DM patient cells throughout the body to a normal functional state.

While illustrative embodiments have been illustrated and described, it will be appreciated that various changes can be made therein without departing from the spirit and scope of the invention.

1. A method of increasing the presence of functional muscleblind-like protein (MBNL) in the nucleus of a cell, comprising contacting the cell with at least one nucleic acid construct comprising a first nucleic acid sequence encoding MBNL and a second nucleic acid sequence encoding a silencing RNA that hybridizes to an mRNA encoding dystrophia myotonica protein kinase (DMPK).

2. The method of claim 1, wherein functional MBNL is not bound to a CTG microsatellite repeat in the 3′ UTR of a nucleic acid encoding DMPK.

3. The method of claim 1 or 2, wherein the MBNL is MBNL1.

4. The method of any one of claims 1 to 3, wherein the cell is a muscle cell.

5. The method of claim 4, wherein the muscle cell is a skeletal muscle cell.

6. The method of any one of claims 1 to 5, wherein the first nucleic acid sequence encodes a non-naturally occurring MBNL protein.

7. The method of claim 6, wherein the non-naturally occurring protein is derived from the MBNL1 gene, optionally wherein the non-naturally occurring protein lacks a functional domain encoded by exon 1 comprising the major part of the 5′UTR and downstream pre-mRNA introns, that could bind MBNL1 protein for autoregulation, of a wild-type muscleblind-like protein 1 mRNA.

8. The method of claim 6, wherein the non-naturally occurring protein is derived from MBNL1 gene and lacks a functional domain encoded by exon 1 comprising the major part of the 5′UTR and downstream MBNL1 pre-mRNA introns, that can bind MBNL1 protein for autoregulation, of a wild-type Muscleblind-like protein 1 gene, and wherein non-naturally occurring protein optionally further lacks a functional domain encoded by intron 2 of the wild-type Muscleblind-like protein 1 gene.

9. The method of any one of claims 1 to 8, wherein the silencing RNA is a microRNA (miRNA), or any small RNA generating and RNAi pathway engaging and activating RNA that, upon hybridizing to the mRNA encoding dystrophia myotonica protein kinase (DMPK), reduces the level of the DMPK mRNA and reduces translation of DMPK protein and cytotoxic proteins, such as repeat-associated non-AUG (RAN) translation products, from the expanded repeat-containing mRNA.

10. The method of any one of claims 1 to 9, wherein the first nucleic acid sequence is operatively linked to a third nucleic acid sequence that is a binding site for a cardiac miRNA.

11. The method of claim 10, wherein the cardiac miRNA is a miRNA expressed in cardiac muscle cells.

12. The method of claim 11, wherein the cardiac miRNA is a miRNA expressed in exclusively or predominantly in cardiac muscle cells.

13. The method of any one of claims 10 to 12, wherein the cardiac miRNA is miR208a.

14. The method of any one of claims 1 to 13, wherein the first nucleic acid sequence is operatively linked to a fourth nucleic acid sequence comprising a chimeric intron with beta-globin (b-globin or β-globin) and immunoglobulin sequences serving as an MBNL1 binding site.

15. The method of any one of claims 1 to 14, wherein the first nucleic acid sequence (and optionally third and/or fourth nucleic acid sequences) and the second nucleic acid sequence are present on separate expression vector constructs.

16. The method of any one of claims 1 to 14, wherein the first nucleic acid sequence (and optionally third and/or fourth nucleic acid sequences) and the second nucleic acid sequence are present on the same expression vector construct.

17. The method of claim 16, wherein the first nucleic acid sequence (and optionally third and/or fourth nucleic acid sequences) and the second nucleic acid sequence are operatively linked to the same promoter, optionally wherein the promoter is CK&e and the like.

18. The method of claim 16, wherein the first nucleic acid sequence (and optionally third and/or fourth nucleic acid sequences) and the second nucleic acid sequence are operatively linked to separate promoter sequences.

19. The method of claim 18, wherein the first nucleic acid sequence (and optionally third and/or fourth nucleic acid sequences) are operatively linked to a CK8e promoter sequence and the second nucleic acid sequence is operatively linked to an RNA Pol III promoter, sequence or RNA Pol II promoter, optionally wherein the RNA Pol III promoter sequence is a U6 promoter sequence, and wherein the RNA Pol II promoter is a CK8 promoter sequence.

20. The method of any one of claims 1 to 19, wherein the one or more nucleic acid constructs are present in a viral vector, e.g., AAV vector.

21. The method of any one of claims 1 to 20, wherein the cell is in vitro.

22. The method of any one of claims 1 to 20, wherein the cell in vivo in a subject with myotonic dystrophy type 1 (DM1), and wherein the method is a method of treating, ameliorating, or preventing symptoms of DM1.

23. The method of claim 22, wherein the subject is a human, rodent (e.g., mouse or rat), dog, cat, and the like.

24. The method of claim 21 or 22, wherein expression of the MBNL protein from the first nucleic acid sequence and the silencing RNA from the second nucleic acid sequence results in an increase in functional MBNL protein in nucleic of skeletal muscle cells in the subject.

25. The method of any one of claims 22 to 24, wherein the first nucleic acid sequence is operatively linked to a third nucleic acid sequence that is a binding site for a cardiac miRNA, wherein the cardiac miRNA is a miRNA expressed in cardiac muscle cells, optionally wherein the cardiac miRNA is miR208a.

26. The method of claim 25, wherein association of the cardiac miRNA to the third nucleic acid sequence prevents or reduces expression of MBNL from the first nucleic acid.

27. A nucleic acid construct comprising:
a first nucleic acid sequence encoding Muscleblind like protein (MBNL); and
a second nucleic acid sequence encoding a silencing RNA that hybridizes to an mRNA encoding dystrophia myotonica protein kinase (DMPK) protein.

28. The nucleic acid construct of claim 27, wherein the first nucleic acid sequence encodes a non-naturally occurring MBNL protein.

29. The nucleic acid construct of claim 28, wherein the non-naturally occurring protein is derived from the MBNL1 gene, optionally wherein the non-naturally occurring protein lacks a functional domain encoded by exon 1 comprising the major part of the 5′UTR and downstream pre-mRNA introns, that could bind MBNL1 protein for autoregulation, of a wild-type muscleblind-like protein 1 mRNA.

30. The nucleic acid construct of claim 28, wherein the non-naturally occurring protein is derived from MBNL1 gene and lacks a functional domain encoded by exon 1 comprising the major part of the 5′UTR and downstream MBNL1 pre-mRNA introns, that can bind MBNL1 protein for autoregulation, of a wild-type Muscleblind-like protein 1 gene, and wherein non-naturally occurring protein optionally further lacks a functional domain encoded by intron 2 of the wild-type Muscleblind-like protein 1 gene.

31. The nucleic acid construct of any one of claims 27 to 30, wherein the first nucleic acid sequence and the second nucleic acid sequence are present in an expression vector and are operatively linked to a first promoter.

32. The nucleic acid construct of any one of claims 27 to 30, wherein the first nucleic acid sequence and the second nucleic acid sequence are present in different expression vectors, wherein the first nucleic acid is operatively linked to a first promoter and second nucleic acid sequence is operatively linked to a second promoter.

33. The nucleic acid construct of claim 31 or 32, wherein the first promoter is active in a skeletal muscle cell.

34. The nucleic acid construct of claim 33, wherein the first promoter is or comprises CK8e and the like.

35. The nucleic acid construct of claim 32, wherein the second promoter is or comprises an RNA Pol III promoter sequence or RNA Pol II promoter sequence, optionally, wherein the RNA pol III promoter is a U6 promoter and the RNApol II promoter is a CK8 promoter.

36. The nucleic acid construct of any one of claims 27 to 35, wherein the nucleic acid construct further comprises a third nucleic acid sequence that is a binding site for a cardiac miRNA, and optionally, wherein the third nucleic acid is operatively linked to the first nucleic acid sequence.

37. The nucleic acid construct of claim 36, wherein the cardiac miRNA is a miRNA expressed in cardiac muscle cells.

38. The nucleic acid construct of claim 37, wherein the cardiac miRNA is a miRNA expressed exclusively or predominantly in cardiac muscle cells.

39. The nucleic acid construct of claim 37, wherein the cardiac miRNA is miR208a.

40. The nucleic acid construct of any one of claims 27 to 39, wherein the nucleic acid construct further comprises a fourth nucleic acid sequence operatively linked to the first nucleic acid sequence, wherein the fourth nucleic acid sequence comprises a chimeric intron with beta-globin (b-globin or β-globin) and immunoglobulin domains.

41. The nucleic acid construct of claim 40, wherein the first nucleic acid sequence and optionally third and/or fourth nucleic acid sequences, and the second nucleic acid sequence are operatively linked to separate promoter sequences.

42. An expression vector comprising a nucleic acid construct, the nucleic acid construct comprising:
a first nucleic acid sequence encoding muscle blind like protein (MBNL);
optionally, a second nucleic acid sequence encoding a silencing RNA that hybridizes to an mRNA encoding dystrophia myotonica protein kinase (DMPK) protein; and
a third nucleic acid sequence operatively linked to the first nucleic acid sequence, wherein the third nucleic acid sequence comprises a binding site for a cardiac miRNA.

43. The expression vector of claim 42, wherein the first nucleic acid sequence is operatively linked to a first promoter that is active in a skeletal muscle cell, and wherein the second nucleic acid sequence is operatively linked to a second promoter.

44. The expression vector of claim 43, wherein the first promoter is or comprises CK8e and the like.

45. The expression vector of any one of claims 42 to 44, wherein the cardiac miRNA is a miRNA expressed in cardiac muscle cells.

46. The expression vector of claim 45, wherein the cardiac miRNA is a miRNA expressed in exclusively or predominantly in cardiac muscle cells.

47. The expression vector of claim 46, wherein the cardiac miRNA is miR208a.

48. The expression vector of any one of claims 42 to 47, wherein the nucleic acid construct further comprises a fourth nucleic acid sequence operatively linked to the first nucleic acid sequence, wherein the fourth nucleic acid sequence comprises a chimeric intron with beta-globin (b-globin or β-globin) and immunoglobulin domains

49. The expression vector of any one of claims 42 to 47, wherein the first nucleic acid sequence and optionally third and/or fourth nucleic acid sequences, and the second nucleic acid sequence are operatively linked to separate promoter sequences

50. The expression vector of any one of claims 42 to 49, wherein the non-naturally occurring protein is derived from the MBNL1 gene, optionally wherein the non-naturally occurring protein lacks a functional domain encoded by exon 1 comprising the major part of the 5′UTR and downstream pre-mRNA introns, that could bind MBNL1 protein for autoregulation, of a wild-type muscleblind-like protein 1 mRNA.

51. The expression vector of any one of claims 42 to 49, wherein the non-naturally occurring protein is derived from MBNL1 gene and lacks a functional domain encoded by exon 1 comprising the major part of the 5′UTR and downstream MBNL1 pre-mRNA introns, that can bind MBNL1 protein for autoregulation, of a wild-type Muscleblind-like protein 1 gene, and wherein non-naturally occurring protein optionally further lacks a functional domain encoded by intron 2 of the wild-type Muscleblind-like protein 1 gene.

52. The expression vector of any one of claims 42 to 51, wherein the expression vector lacks the second nucleic acid sequence.

53. The nucleic acid construct of any one of claims 42 to 52, wherein the first promoter is active in a skeletal muscle cell.

54. The nucleic acid construct of claim 53, wherein the first promoter is or comprises CK8e and the like.

55. The expression vector of any one of claims 42 to 54, wherein the expression vector is a recombinant AAV vector.

56. An expression vector comprising a nucleic acid construct, the nucleic acid construct comprising a second nucleic acid sequence encoding a silencing RNA that hybridizes to an mRNA encoding dystrophia myotonica protein kinase (DMPK) protein, wherein the second nucleic acid sequence is operatively linked to a second promoter.

57. The expression vector of claim 56, wherein the second promoter is or comprises an RNA Pol III promoter sequence or RNA Pol II promoter sequence, optionally, wherein the RNA pol III promoter is a U6 promoter and the RNApol II promoter is a CK8 promoter.

58. The expression vector of any one of claim 56 or 57, wherein the expression vector is a recombinant AAV vector.

59. A pharmaceutical composition comprising the nucleic acid construct of any one of claims 27 to 40 or the expression vector of claims 42 to 58.

60. A method of treating a muscular dystrophy or spliceopathy in a subject in need thereof, the method comprising administering to the subject a therapeutically effective amount of any one of the expression vectors of claims 42 to 58 or at least one of the nucleic acid constructs of claims 27 to 41.


SEQUENCE LISTING (10 sequences)

SEQ ID NO: 1 - protein, 370 aa, synthetic construct
MAVSVTPIRDTKWLTLEVCREFQRGTCSRPDTECKFAHPSKSCQVENGRVIACFDSLKGRCSRENCKYLHPPPHLKTQLEINGRNNLIQQKNMAMLAQQMQLANAMMPGAPLQPVPMFSVAPSLATNASAAAFNPYLGPVSPSLVPAEILPTAPMLVTGNPGVPVPAAAAAAAQKLMRTDRLEVCREYQRGNCNRGENDCRFAHPADSTMIDTNDNTVTVCMDYIKGRCSREKCKYFHPPAHLQAKIKAAQYQVNQAAAAQAAATAAAMGIPQAVLPPLPKRPALEKTNGATAVFNTGIFQYQQALANMQLQQHTAFLPPVPMVHGATPATVSAATTSATSVPFAATATANQIPIISAEHLTSHKYVTQM

SEQ ID NO: 2 - protein, 311 aa, synthetic construct
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 3 - DNA, 8656 bp, synthetic construct
  source 1..8656: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacaaactctagagcggccgcacgcgtacgctagctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtatacgtacttctgacacaacagtctcgaacttaagctgcagaagttggtcgtgaggcactgggcaggtaagtatcaaggttacaagacaggtttaaacggagaccaatagaaactgggcttgtcgagacagagaagactcttgcgtttctgataggcacctattggtcttactgacatccactttgcctttctctccacaggtgtccactcccagttcaattacagctcttaaggctagagtaaccggtgccaccatggcagttagtgttactccgatacgcgataccaagtggctcactctcgaagtctgtagagaatttcagcgagggacctgctcccgacctgatactgaatgtaagtttgcgcacccatctaaatcatgtcaagtggagaacggcagggtgattgcatgtttcgacagtctcaaaggccggtgttcacgagagaattgcaagtatcttcatccaccaccgcacctcaagactcagcttgaaataaacggccgcaataaccttatacaacagaagaacatggccatgctggcacaacaaatgcaattggccaacgcaatgatgccaggggcgcccctgcaaccagttccaatgttctcagttgcaccatcactcgcaacaaatgcatccgctgctgccttcaacccgtaccttgggcccgtttcacccagccttgtgcccgctgagattctccctacagcgcctatgctggtaacgggtaaccctggtgtccccgttcccgcagcagcggccgcagccgctcagaaattgatgcgcaccgacagattggaggtatgccgagagtaccagcgcggaaactgtaatagaggggaaaatgactgccggtttgcccacccagctgactcaacaatgattgacactaatgacaatacggttaccgtttgtatggactacataaaaggacggtgctcccgcgaaaaatgcaaatacttccatcccccagcgcatcttcaggccaagattaaggcagcccaataccaagtcaaccaggcagcggctgcgcaagcagcagccacggcagcagcaatgggcattccgcaggcagtactcccaccccttccaaagaggccggcgctcgagaagaccaatggtgcaacagctgtgtttaacacgggtatctttcaatatcagcaagcacttgcgaacatgcaactccaacaacatactgcgtttttgccaccagtcccaatggtacatggcgctacaccggcgacagtaagtgctgcgacaacgagcgctacatccgtgccattcgctgcgacagctacagcgaaccagatcccgatcatctccgcagagcacttgacttcccataagtacgttactcagatgtagtccggagttaaccctaggggcgcgcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataattgtacacctgcaggcacatcggtggacctgagtcacaagctttttgctcgtcttatgtcgaccctcgaggacaagctttttgctcgtcttatgtcgacgacctgagtcacaagctttttgctcgtcttatcacatcggtgcctgcaggtgtacaagggaacttgtttattgcagcttataatggttacaaataaagcaatagcatcacaaatttcacaaataaagcatttttttcactgcagatctgaattggccgtttgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgatttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtggggtacttatataagggggtgggggcgcgttcgtcctcagtcgcgatcgaacactcgagccgagcagacgtgcctacggaccgtaatacgactcactatagggaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagggggatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 4 - DNA, 8508 bp, synthetic construct
  source 1..8508: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacaaactctagagcggccgcacgcgtacgctagctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtatacgtacttctgacacaacagtctcgaacttaagctgcagaagttggtcgtgaggcactgggcaggtaagtatcaaggttacaagacaggtttaaacggagaccaatagaaactgggcttgtcgagacagagaagactcttgcgtttctgataggcacctattggtcttactgacatccactttgcctttctctccacaggtgtccactcccagttcaattacagctcttaaggctagagtaaccggtgccaccatggcagttagtgttactccgatacgcgataccaagtggctcactctcgaagtctgtagagaatttcagcgagggacctgctcccgacctgatactgaatgtaagtttgcgcacccatctaaatcatgtcaagtggagaacggcagggtgattgcatgtttcgacagtctcaaaggccggtgttcacgagagaattgcaagtatcttcatccaccaccgcacctcaagactcagcttgaaataaacggccgcaataaccttatacaacagaagaacatggccatgctggcacaacaaatgcaattggccaacgcaatgatgccaggggcgcccctgcaaccagttccaatgttctcagttgcaccatcactcgcaacaaatgcatccgctgctgccttcaacccgtaccttgggcccgtttcacccagccttgtgcccgctgagattctccctacagcgcctatgctggtaacgggtaaccctggtgtccccgttcccgcagcagcggccgcagccgctcagaaattgatgcgcaccgacagattggaggtatgccgagagtaccagcgcggaaactgtaatagaggggaaaatgactgccggtttgcccacccagctgactcaacaatgattgacactaatgacaatacggttaccgtttgtatggactacataaaaggacggtgctcccgcgaaaaatgcaaatacttccatcccccagcgcatcttcaggccaagattaaggcagcccaataccaagtcaaccaggcagcggctgcgcaagcagcagccacggcagcagcaatgggcattccgcaggcagtactcccaccccttccaaagaggccggcgctcgagaagaccaatggtgcaacagctgtgtttaacacgggtatctttcaatatcagcaagcacttgcgaacatgcaactccaacaacatactgcgtttttgccaccagtcccaatggtacatggcgctacaccggcgacagtaagtgctgcgacaacgagcgctacatccgtgccattcgctgcgacagctacagcgaaccagatcccgatcatctccgcagagcacttgacttcccataagtacgttactcagatgtagtccggagttaaccctaggggcgcgcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataattgtacaagggaacttgtttattgcagcttataatggttacaaataaagcaatagcatcacaaatttcacaaataaagcatttttttcactgcagatctgaattggccgtttgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgatttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtggggtacttatataagggggtgggggcgcgttcgtcctcagtcgcgatcgaacactcgagccgagcagacgtgcctacggaccgtaatacgactcactatagggaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagggggatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 5 - DNA, 8739 bp, synthetic construct
  source 1..8739: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacaaactctagagcggccgcacgcgtacgctagctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtatacgtacttctgacacaacagtctcgaacttaagctgcagaagttggtcgtgaggcactgggcaggtaagtatcaaggttacaagacaggtttaaacggagaccaatagaaactgggcttgtcgagacagagaagactcttgcgtttctgataggcacctattggtcttactgacatccactttgcctttctctccacaggtgtccactcccagttcaattacagctcttaaggctagagtaaccggtgccaccatggcagttagtgttactccgatacgcgataccaagtggctcactctcgaagtctgtagagaatttcagcgagggacctgctcccgacctgatactgaatgtaagtttgcgcacccatctaaatcatgtcaagtggagaacggcagggtgattgcatgtttcgacagtctcaaaggccggtgttcacgagagaattgcaagtatcttcatccaccaccgcacctcaagactcagcttgaaataaacggccgcaataaccttatacaacagaagaacatggccatgctggcacaacaaatgcaattggccaacgcaatgatgccaggggcgcccctgcaaccagttccaatgttctcagttgcaccatcactcgcaacaaatgcatccgctgctgccttcaacccgtaccttgggcccgtttcacccagccttgtgcccgctgagattctccctacagcgcctatgctggtaacgggtaaccctggtgtccccgttcccgcagcagcggccgcagccgctcagaaattgatgcgcaccgacagattggaggtatgccgagagtaccagcgcggaaactgtaatagaggggaaaatgactgccggtttgcccacccagctgactcaacaatgattgacactaatgacaatacggttaccgtttgtatggactacataaaaggacggtgctcccgcgaaaaatgcaaatacttccatcccccagcgcatcttcaggccaagattaaggcagcccaataccaagtcaaccaggcagcggctgcgcaagcagcagccacggcagcagcaatgggcattccgcaggcagtactcccaccccttccaaagaggccggcgctcgagaagaccaatggtgcaacagctgtgtttaacacgggtatctttcaatatcagcaagcacttgcgaacatgcaactccaacaacatactgcgtttttgccaccagtcccaatggtacatggcgctacaccggcgacagtaagtgctgcgacaacgagcgctacatccgtgccattcgctgcgacagctacagcgaaccagatcccgatcatctccgcagagcacttgacttcccataagtacgttactcagatgtagtccggagtttcgagtgagcgcggcttaaggaggtccgattgactgtaaagccacagatgcttagtcggacctccttaagccacgcctactagaaccctaggggcgcgcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataattgtacacctgcaggcacatcggtggacctgagtcacaagctttttgctcgtcttatgtcgaccctcgaggacaagctttttgctcgtcttatgtcgacgacctgagtcacaagctttttgctcgtcttatcacatcggtgcctgcaggtgtacaagggaacttgtttattgcagcttataatggttacaaataaagcaatagcatcacaaatttcacaaataaagcatttttttcactgcagatctgaattggccgtttgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgatttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtggggtacttatataagggggtgggggcgcgttcgtcctcagtcgcgatcgaacactcgagccgagcagacgtgcctacggaccgtaatacgactcactatagggaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagggggatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 6 - DNA, 8413 bp, synthetic construct
  source 1..8413: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacaaactctagagcggccgcacgcgtacgctagctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtaccggtgccaccatggcagttagtgttactccgatacgcgataccaagtggctcactctcgaagtctgtagagaatttcagcgagggacctgctcccgacctgatactgaatgtaagtttgcgcacccatctaaatcatgtcaagtggagaacggcagggtgattgcatgtttcgacagtctcaaaggccggtgttcacgagagaattgcaagtatcttcatccaccaccgcacctcaagactcagcttgaaataaacggccgcaataaccttatacaacagaagaacatggccatgctggcacaacaaatgcaattggccaacgcaatgatgccaggggcgcccctgcaaccagttccaatgttctcagttgcaccatcactcgcaacaaatgcatccgctgctgccttcaacccgtaccttgggcccgtttcacccagccttgtgcccgctgagattctccctacagcgcctatgctggtaacgggtaaccctggtgtccccgttcccgcagcagcggccgcagccgctcagaaattgatgcgcaccgacagattggaggtatgccgagagtaccagcgcggaaactgtaatagaggggaaaatgactgccggtttgcccacccagctgactcaacaatgattgacactaatgacaatacggttaccgtttgtatggactacataaaaggacggtgctcccgcgaaaaatgcaaatacttccatcccccagcgcatcttcaggccaagattaaggcagcccaataccaagtcaaccaggcagcggctgcgcaagcagcagccacggcagcagcaatgggcattccgcaggcagtactcccaccccttccaaagaggccggcgctcgagaagaccaatggtgcaacagctgtgtttaacacgggtatctttcaatatcagcaagcacttgcgaacatgcaactccaacaacatactgcgtttttgccaccagtcccaatggtacatggcgctacaccggcgacagtaagtgctgcgacaacgagcgctacatccgtgccattcgctgcgacagctacagcgaaccagatcccgatcatctccgcagagcacttgacttcccataagtacgttactcagatgtagtccggagttaaccctaggggcgcgcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataattgtacacctgcaggcacatcggtggacctgagtcacaagctttttgctcgtcttatgtcgaccctcgaggacaagctttttgctcgtcttatgtcgacgacctgagtcacaagctttttgctcgtcttatcacatcggtgcctgcaggtgtacaagggaacttgtttattgcagcttataatggttacaaataaagcaatagcatcacaaatttcacaaataaagcatttttttcactgcagatctgaattggccgtttgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgatttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtggggtacttatataagggggtgggggcgcgttcgtcctcagtcgcgatcgaacactcgagccgagcagacgtgcctacggaccgtaatacgactcactatagggaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagggggatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 7 - RNA, 2888 nt, synthetic construct
  source 1..2888: mol_type = other RNA
ggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggccccaaatccagggttttccaaagtgtggttcaagaaccacctgcatctgaatctagagcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccagcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccacggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactcaggggccccgttggaagactgagtgcccggggcacggcacagaagccgcgcccaccgcctgccagttcacaaccgctccgagcgtgggtctccgcccagcaccagtcctgtgatccgggcccgccccctagcggccggggagggaggggccgggtccgcggccggcgaacggggctcgaagggtccttgtagccgggaatgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttacccgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaaaaaaaaaaaaaaa

SEQ ID NO: 8 - RNA, 2768 nt, synthetic construct
  source 1..2768: mol_type = other RNA
gggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaaaaaaaaaaaaaaaaaaaaaaaaaaaa

SEQ ID NO: 9 - RNA, 5516 nt, synthetic construct
  source 1..5516: mol_type = other RNA
agtcttttcactgcagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaagtaagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccaggctcaatattgtgcatgacacccgctacaagtgttgttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtagaattttcatcactaaacaatcatgctaaagaggaaaggacagtgtgcttggttagagtaaaggacgaggtcattagccatattgtatatatcgtcaagcaacacacacaaaagttcctcagccacaagacatccacatattgcatgttaaccagaagaaaagacaacattttccggaaatccactgcacactgttgcctatacactttgtacatttaattgatatttgtgctgaggtgatattcctgtctaaaagaacaacattgtctttcttttctagcacagagttatgcattcaaagatgcatacctagttagtttcctatatattcatgccatcttgaaaagacagactatggtgtaaccatgattctattatgtattggtacgtctgtagaccaagatataattttttaaaaataagtttatttctttcaaggtttacaaataacaaaggtgcaccttgtatttaaaattgccattatagatgagagcgtgcatgcacagtcatttttgtttaagagtaatatttttaatgtaatagattgtaagacgtggtgagggagggatctgacagagatgaatgtgccaagcaaaaccacaactgtgtatattttaaagcacatcatggctttaagtaccatgttgttaaggattctcatgaagtgccatagactgtacatcaaattagagtattatttcttcagtgttattgttttcagagccacattttgttgcatatttgctagtactaatcagtcaaagggcaccattctttttttttttttttgaaaccaaagctgtctcagaaatggccaatttaactttacagtaacaatagacagcacaacacaaactctctcaatacagataaactcacacatactggagatatatatataatagatatatataaaattattttaatgcattgtagtgtaatatttatgcatactatactgtataacatgttattcaaaagggattgccatttctgagacacagtaacaaaaaaatgaggaaattattttgcttctatttatagcctctgtcaaaagtcaaaagactataaatgctttgcaaaaatggtttcacgtttgcttaaatgcttcatcacagtcacattcaaaatagtgactctaaacaaagaagaaagcagcactgtcatcagatgcatgataaaccaaaatatgaaaatgggaaatgtttaattaacctagtaattgggtgggttaagtacatgggtgaattttatatgtgatttttgttttgttttgttttgttcagattaactgcttatagccttagaaagccttttacaaaattaaaaaaaaaatagatgtgcattcagtttttaagaatggaatcatccaaaggaattcctttttttgaggtttggatgttgcagctagtaaaggatatttttgctctgttcagcagttctaaaaattgctgaagtaggggccaggtcactggtagttatagtatggaatgggagaagtgaaagttcagttatagaactttccatacttccaagtttactgcaagtttttatgcttgagagagatgctttctaatataagactgatgtgttgattttactgattgtactgtacatctattaaagccttagattattacattacgggttggaacccataccaatgtaatttcaatcgtgttaagaaagtaatggtgacttcacatgttattgtagttagttacattatagaatattacttatttttcttgttaaaatgtagtttttcatttcctacatttattagattttcattttctattaacaattgaataccatttcagtttatagacttgttttattagattttaccaatgaatttttcaaaatacaaaaaaaagtagtttttccttcataacatactcagttttgaattacatgtagtgtcacatgaatattcgtattgttaactaaatgatttatattttactgatttaatattacagtgtaagaatgtcagtcattgttagttcttgtctagttttcattaaaagaacaaagatcttttatatggatatcttataaatatataatcattgctaagtaagaagttaagttgttgctatcgcaacaatcctggcagacaattgagtaatattttgatgatttattttgtttgtaattagttattataagaagatctagatcctagatattagaataaaatttattttctactgtatccatttcaaatgttaaaatattgtttaatatttttgaaatccctgagtatcaggccttgttataaataagctgcataatcaataaatagaacaagggactttttgttgataatccaaatactcaaagtttacgtaatgaaaattatagcgtgtgtgcaaactcttgagggttgattatgctgcaatttagcatgttggaacgtctagggagaaggttgactttttgcacttctgtatatagtcaaaagagagaaacctgtataatagtaagatcttattttgaataaaaacgtctataattacaaggagttttgttaaggctaatacaatgacagactgagcaaaattgcttgcaaaagtggcacagagttagcactccataccccttcaaacatgttgctttgctttcttgtggacagcttgtagtttgccaggattttttcagctggaaagatacgccatcctttcaaaccctcatgactgacaaaaactccatggggccaaatctgcctgaagatcattaccaaaaatagcaggtacttctaccattaaggtgaaatcatggatcagatattccttacatttttcaaaactactgcatgtttaaaacttcaacaaaaaaagagagaaagaactatactaagaacatatattattcagatcagtttctgccaatttcagtggtttattgttcacaaaaaaatcttcaaaacaagtattgactttcacaaaatttaaatcataaacaggcaaaccaaacagcacactgtagctatagttgttatgtgattgttttttaattgctgtaggatcctgttctttcagcaggtgaaaaataaaacgcagttcaaatttcatggttttaattttcaactcagaagcactcaaaaatgcaaaatgtgataatgggcacttgtttaaaagaattagtgtatccagccttcactccagctggttaaaaatgttgcacttatcagcaaccctaccactttcatctgctgaaaggacaaatgtgcttggttttactattatgtaatcacaacttactttctgcttgtagttgcttaaaattatgtattttgtcttgggctgcaatttgttttatgcttattttattattactgcagtagttgactttgctgtatggaaaaataaagtgaaattgccctaataaaacttctctttcttaagta

SEQ ID NO: 10 - RNA, 4650 nt, synthetic construct
  source 1..4650: mol_type = other RNA
agactgtctttgcttcatcatctgaaggtaaaattttccagatacggcagacggctttcagagtacaataaacagggaatgagaactatttacatggaagtttctttctcatgatgcggtggagaagcctcggccacttggttctgccagatgttcctggggttactgtaaatgggaaggacaggcagagctaaacaaggtttatcatttaaaagtgcctgtgtgaagtcacttttgctggaaaactgcagcttgggagctttctttgtattcacatcccactcttctgtcaagtacactttaccctgaccttatgagtggatgaagatacctcagttgtctgactttgccaattgcttaatttcagaatttaaaaaggggaaagaaaaacatcctgctaaaatatgaacatctgagtgtcttattttccaacatcgtcaatagctgtgagcgtcagcattaaatattctcccaaggagtgccatgatattgaagtcactttattaataacagctgtatctgcaaaacagtcaagagactcggacgttgaaagccagagatgacactgagcatgcttttattgcggcctaccatctttaagtgggacatattgattgatgagtgattgcctgtccatacactctctcatcatcctgttccttggattggacttcactaagcaatttatcactcaccttcagacttacatgtgggagttttcacaacagtagttttggaatcattagaacttggattgatttcatcatttaacagaaacaaacagcccaaattactttatcaccatggctttgaacgttgccccagtcagagatacaaaatggctgacattagaagtctgcagacagtttcaaagaggaacatgctcacgctctgatgaagaatgcaaatttgctcatccccccaaaagttgtcaggttgaaaatggaagagtaattgcctgctttgattccctaaagggccgttgttcgagagagaactgcaagtatcttcaccctccgacacacttaaaaactcaactagaaattaatggaaggaacaatttgattcagcaaaaaactgcagcagcaatgcttgcccagcagatgcaatttatgtttccaggaacaccacttcatccagtgcccactttccctgtaggtcccgcgatagggacaaatacggctattagctttgctccttacctagcacctgtaacccctggagttgggttggtcccaacggaaattctgcccaccacgcctgttattgttcccggaagtccaccggtcactgtcccgggctcaactgcaactcagaaacttctcaggactgacaaactggaggtatgcagggagttccagcgaggaaactgtgcccggggagagaccgactgccgctttgcacaccccgcagacagcaccatgatcgacacaagtgacaacaccgtaaccgtttgtatggattacataaaggggcgttgcatgagggagaaatgcaaatattttcaccctcctgcacacttgcaggccaaaatcaaagctgcgcagcaccaagccaaccaagctgcggtggccgcccaggcagccgcggccgcggccacagtcatgactcagtcgactgccaaagcaatgaagcgacctctcgaagcaactgtagacctggcctttccccctggtgctcttcatcctttaccaaagagacaagcacttgaaaaaagcaatggtaccagcgcggtctttaaccccagcgtcttgcactaccagcaggctctcaccagcgcacagttgcagcaacacgccgcgttcattccaacagggtcagttttgtgcatgacacccgctaccagtattgataattctgaaataatcagcagaaacggaatggaatgccaagaatctgcattgagaataactaaacattgttactgtacatactatcctgtttcctcctcaatagaattgccacaaactgcatgctaaataaagatgtagttcttctggacagaccacaactctaagaagctagtgctgctatctcatatatgagtattaaatatggtatgcttagtatattccaacctaagatagttaactacctgagaccagctgtgatgtttaaagacataaaggataaagtttacttttaaagggtttctaaacatagtttctgtcctaggaatattgtcttatctccataactatagctgatgcagaaagtccagccagtttactcatttcgattcagaatatttcaaatttagcaataaacaattagcattagttaaaaaagaaacatattccaagggcaggttcgattctagctctaattactgtcatgtcatttacccactggatcaaagggtatgtttcacttcttgacaatataaatgctgcagcaaagatgagaggtgaagtaaaaccgatacctgtcctgcaggtctaaaatttgaatggaaattcaagcacaagtactggggacacatcaaagtgtggtgtttggtttgcctggagatgccacgttgaatcatgtgattctagattaacattaaatagattgaaaaagaaactttgcacggtatgagcttcataccccaccaaacaaagtcttgaaggtattattttacaagtatatttttaaagttgttttataagagagactttgtagaagtgcctagattttgccagacttcatccagcttgacaagattgagaggcccatgccaacagtctaatctaagagattagtctttcaaactcaccatccagttgcctgttacagaataactcttcttaactaaaaacctagtcaaacaaggaagctgtaggtgaggagatctgtataatattctaatttaagtaagtttgagtttagtcactgcaaatttgactgtgactttaatctaaattactatgtaaacaaaaagtagatagtttcactttttaaaaaatccattactgttttgcatttcaaaagttggattaaagggttgtaactgactacagcatggaaaaaaatagttcttttaattctttcaccttaaagcatattttatgtctcaaaagtataaaaaactttaatacaagtacatacatattatatatacacatacatatatatactatatatggatgaaacatattttaatgttgtttacttttttaaatacttggttgatcttcaaggtaatagcgatacaattaaattttgttcagaaagtttgttttaaagtttattttaagcactatcgtaccaaatatttcatatttcacattttatatgttgcacatagcctatacagtacctacatagtttttaaattattgtttaaaaaacaaaacagctgttataaatgaatattatgtgtaattgtttcaaacatccattttctttgtgaacatattagtgattgaagtattttgacttttgagattgaatgtaaaatattttaaatttgggatcatcgcctgttctgaaaactagatgcaccaaccgtatcattatttgtttgaggaaaaaaagaaatctgcattttaattcatgttggtcaaagtcgaattactatctatttatcttatatcgtagatctgataaccctatctaaaagaaagtcacacgctaaatgtattcttacatagtgcttgtatcgttgcatttgttttaatttgtggaaaagtattgtatctaacttgtattactttggtagtttcatctttatgtattattgatatttgtaattttctcaactataacaatgtagttacgctacaacttgcctaaaacattcaaacttgttttcttttttctgtttttttctttgttaattcatttaaactcattgaaaacatagtatacattactaaaaggtaaattatgggaatcactgaaatatttttgtagattaattgttgtaacattgtctttcttttttttcttttgtttcatgattttgatttttaaaattattagcacacaactattttcagccctttaataatggagcatcaaaaacatcacctgtaaccccaagcaaatatagaagactgtattttttactatgatatccattttccagaattgtgattacaatatgcaaagagtcataaatatgccatttacaataaggaggaggcaaggcaaatgcatagatgtacaaatatatgtacaacagattttgctttttatttatttataatgtaattttatagaataattctgggatttgagaggatctaaaactatttttctgtataaatattatttgccaaaagtttgtttatattcagaagtctgactatgatgaataaatcttaaatgctttgtttaattaaaaaacaaaaatcaccaatatccaagacatgaagatatcagttcaacaaatactgtagttaagagactaactctccacttgtatgggaactacatttcactcttggttttcaggatataacagcacttcaccgaaatattctttcagccataccactggtaacatttctactaaatctttctgtaacacttaaagaattccctcattcattaccttacagtgtaaacaggagtctaatttgtatcaatactatgttttggttgtaatattcagttcactcacccaatgtacaaccaatgaaataaaagaagcatttaaaagga